(19) United States
(12) Patent Application Publication
(10) Pub. No.: US 2010/0068804 A1
(43) Pub. Date: Mar. 18, 2010

(54) METHODS OF STIMULATING EXPANSION OF HEMATOPOIETIC STEM CELLS

(75) Inventor: John P. Chute, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 11/991,015

(22) PCT Filed: Sep. 1, 2006
(86) PCT No.: PCT/US2006/034330

§ 371 Date: Dec. 1, 2009

Related U.S. Application Data
(60) Provisional application No. 60/712,829, filed on Sep. 1, 2005.

(51) Int. Cl. C12N 5/071; C12N 5/02

(52) U.S. Cl. 435/366; 435/375

(57) ABSTRACT

The present invention relates, in general, to hematopoietic stem cells (HSC) and, in particular, to methods of stimulating expansion of hematopoietic stem cells and to agents suitable for use in such methods.

This application claims priority from U.S. Provisional Application No. 60/712,829, filed Sep. 1, 2005, the entire content of which is incorporated herein by reference.

### TECHNICAL FIELD

The present invention relates, in general, to hematopoietic stem cells (HSC) and, in particular, to methods of stimulating expansion of hematopoietic stem cells and to agents suitable for use in such methods.

### BACKGROUND

Cell surface expression of the CD34 antigen is a reliable indicator of enrichment for hematopoietic progenitor and stem cells (Link et al, Blood 87:4903-4909 (1996), Civin and Gore, J. Hematother. 93:2217-2224 (1993)). However, cells within the CD34⁺ compartment are heterogeneous and include committed CD34⁺CD38⁺ progenitors which lack stem cell activity (Hao et al, Blood 86:3745-3753 (1995), Bhatia et al, Proc. Natl. Acad. Sci. USA 94:5320-5325 (1997)). The CD34⁺CD38⁻ fraction makes up 1-10% of the CD34⁺ population and is highly enriched for both extended long term culture-initiating cells (ELTC-ICs) and the most primitive assayable cells which are capable of repopulating Nonobese Diabetic/Severe Combined Immuno-Deficient (NOD/SCID) mice (SCID Repopulating Cells, SRC) (Hao et al, Blood 86:3745-3753 (1995), Bhatia et al, Proc. Natl. Acad. Sci. USA 94:5320-5325 (1997), Larochelle et al, Nat. Med. 2:1329-1337 (1996), Shah et al, Blood 87:3563-3570 (1996), Leemhuis et al, Exp. Hematol. 24:1215-1224 (1999)). Using fluorescence activated cell sorting to collect steady state cord blood CD34⁺ CD38⁻ cells, Bhatia et al (Proc. Natl. Acad. Sci. USA 94:5320-5325 (1997)) demonstrated a frequency of 1 SRC per 617 cord blood CD34⁺CD38⁻ cells and no detectable SRC within the CD34⁺CD38⁺ fraction (Bhatia et al, Proc. Natl. Acad. Sci. USA 94:5320-5325 (1997)). Bhatia et al, J. Exp. Med. 186:619-624 (1997)) also showed, via a limiting dilution analysis, that purified human cord blood (CB) CD34⁺CD38⁻ cells could be cultivated ex vivo with cytokines resulting in a 2-4 fold increase in SRC at day 4 followed by loss of SRC by day 9. A subsequent study by Glimm et al (Blood 94:2161-2168 (1999)) further demonstrated that self-renewal divisions occur within primitive cord blood repopulating cells during 5 day cytokine suspension cultures. Other studies have suggested that human CB SRC can be maintained ex vivo in liquid suspension cultures from 1-12 weeks (Conneally et al, Proc. Natl. Acad. Sci. USA 94:9836-9841 (1997), Piacibello et al, Blood 93:3736-3749 (1999), Tanavde et al, Exp. Hematol. 30:816-823 (2002)). Conversely, ex vivo culture of adult (BM, peripheral blood) sources of human CD34⁺ stem cells with cytokines, with and without stroma, has been reproducibly associated with the loss of primitive repopulating cells over time Gan et al (Blood 90:641-650 (1997), Gothot et al, Blood 92:2641-2649 (1998), Chute et al, Blood 100:4433-4439 (2002), Haylock et al, Blood 80:1405-1412 (1992), Sato et al, Blood 82:3600-3609 (1993)). Investigations into the proliferative capacity and SRC content of purified human BM CD34⁺CD38⁻ cells have been more limited in part due to a lack of culture conditions which support the maintenance or expansion of adult HSC (Bhatia et al, J. Exp. Med. 186:619-624 (1997), Bennaceur-Griselli et al, Blood 97:435-441 (2001)).

Recent studies have indicated that expression of CD38 antigen on CD34⁺CD38⁺ hematopoietic progenitors may be down-modulated during ex vivo culture with cytokines, calling into question the reliability of the CD34⁺CD38⁻ phenotype as an indicator of HSC content during or post-culture (Dorrell et al, Blood 95:102-110 (2000), Danet et al, Exp. Hematol. 29:1465-1473 (2001)). As evidence that the CD34⁺CD38⁻ phenotype did not correlate with primitive stem cell content, Dorrell et al. demonstrated that surface expression of myeloid Maturation antigens, CD33 and CD13, increased more than 2-fold on CB “CD34⁺CD38⁻” cells during short-term ex vivo culture (Dorrell et al, Blood 95:102-110 (2000)). This down-regulation of surface CD38 expression on committed progenitors has been attributed to a depletion of retinoids which occurs over time during in vitro culture (Mehta et al, Blood 89:3607-3614 (1997), Drach et al, Cancer Res. 54:1746-1752 (1994)). It was recently reported that co-culture of adult human BM CD34⁺ cells with primary human brain endothelial cells (HUBEC) induced a 4.1-fold increase in SRC frequency (Chute et al, Blood 100:4433-4439 (2002)), but also observed was a significantly larger expansion (212-fold) of cells bearing the CD34⁺CD38⁻ phenotype (Chute et al, Blood 100:4433-4439 (2002)). Conversely, it was found that the loss of phenotypic CD34⁺CD38⁻ cells following liquid suspension culture with GMCSF, IL-3, IL-6, SCF, and Flt-3 ligand alone was associated with a complete loss of SRC (Chute et al, Blood 100:4433-4439 (2002)). Taken together, these data suggested that SRC may have been contained within the CD34⁺CD38⁻ fraction following HUBEC-culture, but this was not measurable since heterogeneous CD34⁺ cells were utilized in these studies.

Since the ability to conveniently monitor HSC content during/post-ex vivo culture would be advantageous for both research and clinical purposes, it was important to define the phenotype of adult HSC which had replicated under HSC-supportive culture conditions. The present invention results, at least in part, from rigorous cell sorting experiments which demonstrated that SRC expansion correlates with amplification of BM CD34⁺CD38⁻ cells and that this result appears to be mediated by soluble factors elaborated by human brain endothelial cells. The invention further results from studies identifying endothelial-derived proteins that stimulate the expansion of human HSC.

### SUMMARY OF THE INVENTION

In general, the present invention relates to hematopoietic stem cells. More specifically, the invention relates to methods of stimulating expansion of hematopoietic stem cells, particularly human hematopoietic stem cells, and to agents (e.g., endothelial-derived proteins) suitable for use in such methods.

Objects and advantages of the present invention will be clear from the description that follows.

### BRIEF DESCRIPTION OF THE DRAWINGS

FIGS. 1A-1C. Phenotypic analysis of unsorted BM CD34⁺ cells at day 0 and following HUBEC culture versus GM36SF alone. Human BM CD34⁺ cells were cultured with either HUBEC monolayers supplemented with GM36SF or GM36SF alone for 7 days (n=3). (FIG. 1A) Representative phenotype of BM CD34⁺ cells at day 0, day 4, and day 7 of HUBEC culture, demonstrating a high percentage of CD34⁺CD38⁻ cells persistent post-culture. (FIG. 1B) Representative phenotype of BM CD34⁺ cells at day 0, day 4, and day 7 of culture with GM36SF alone, demonstrating nearly complete loss of CD34⁺CD38⁻ cells. All cell populations were stained with anti-CD34 FITC and anti-CD38 PE antibodies and analyzed by flow cytometry. Isotype control staining for each time point is shown at top (FIG. 1C).

FIGS. 2A and 2B. Phenotypic response of purified BM CD34⁺CD38⁻ and CD34⁺CD38⁺ cells to ex vivo culture with HUBEC. (FIG. 2A) A representative experiment showing the phenotypic changes which occurred within FACS-sorted BM CD34⁺CD38⁻ cells over 7 days of culture with HUBEC+GM36SF. Note that the majority of day 7 HUBEC-cultured cells were either CD34⁺CD38⁻ or CD34⁻CD38⁻ (n=8). (FIG. 2B) A representative experiment showing the phenotypic changes which occurred within FACS-sorted BM CD34⁺CD38⁺ cells over 7 days of co-culture with HUBEC+GM36SF. At day 7, the majority of the cells are CD34⁻CD38⁻, but a minor population of CD34^{dim}CD38⁻ cells remains.

FIGS. 3A-3D. HUBEC non-contact cultures and HUBEC+TSF support the differential maintenance of CD34⁺CD38⁻ cells. BM CD34⁺CD38⁻ cells were placed in 7 day cultures to determine phenotype changes over time (n=3 each). (FIG. 3A) Following 7 day non-contact culture with HUBEC+GM36SF, a high percentage of CD34⁺CD38⁻ cells persisted over time. (FIG. 3B) Following 7 day culture with GM36SF alone, significant losses of CD34⁺CD38⁻ cells were observed. (FIG. 3C) Following 7 day culture with HUBEC+TSF, the majority of cells remained CD34⁺CD38⁻ post-culture. (FIG. 3D) Conversely, at day 7 of culture with TSF alone, the majority of the input CD34⁺CD38⁻ population was lost.

FIG. 4. HUBEC co-culture significantly increases the SRC frequency within human BM CD34⁺CD38⁻ cells. The scatter plot shows the level of human CD45⁺ cell engraftment in NOD/SCID mice at week 8 following transplantation with FACS-sorted BM CD34⁺CD38⁻ cells or their progeny as indicated. The progeny of 2-4×10⁴ BM CD34⁺CD38⁻ cells cultured×7 days with HUBEC+GM36SF showed significantly higher levels of human engraftment compared to day 0 BM CD34⁺CD38⁻ cells at the same dose. As shown at right, the highest human CD45⁺ cell engraftment was observed in mice transplanted with the progeny of 4×10⁴ BM CD34⁺CD38⁻ cells following non-contact HUBEC cultures+GM36SF. Mice transplanted with the progeny of BM CD34⁺CD38⁻ cells cultured with HUBEC+TSF displayed comparable engraftment to non-contact HUBEC+GM36SF cultures. Each circle represents an individual mouse transplanted with human BM cells. Values on the Y axis indicate the percentage of human CD45⁺ cell engraftment within the marrow of individual NOD/SCID transplanted mice. The cell dosages for each group are shown at top. The mean levels of human CD45⁺ cell engraftment are indicated by horizontal bars for each group.

FIGS. 5A and 5B. Human SRC are enriched within the CD34⁺CD38⁻ subset following expansion divisions. FACS-sorted BM CD34⁺CD38⁻ cells (4×10⁴) were cultured×7 days with HUBEC+GM36SF. At day 7, the progeny of culture were collected, stained with anti-CD34 FITC and anti-CD38 PE and flow cytometric analysis and cell sorting was performed. As shown in (FIG. 5A), sterile FACS sorting of day 7 CD34⁺CD38⁻ and CD34⁻CD38⁻ cell subsets was performed and each population was collected separately. NOD/SCID mice (n=7 per group) were transplanted with the collected cell subsets and human CD45⁺ cell engraftment was measured after 8 weeks. SRC activity was detected only within the day 7 CD34⁺CD38⁻ population, whereas SRC activity was not demonstrable within the day 7 CD34⁻CD38⁻ subset. In (FIG. 5B), the lineage distribution of engrafted human cells is shown within a representative mouse 8 weeks post-transplantation with day 7 FACS-sorted CD34⁺CD38⁻ cells. At top left, no huCD45⁺ cell engraftment is demonstrable within a representative mouse transplanted with Day 7 CD34⁻CD38⁻ cells. At top right, huCD45⁺ cell engraftment is evident in a mouse transplanted with day 7 FACS-sorted CD34⁺CD38⁻ cells. CD34⁺ progenitor cell engraftment (middle left), CD19⁺ B cell differentiation (middle right), and CD13⁺ myeloid differentiation (lower left) are also shown.

FIG. 6. Assessment of the linear models by a volcano plot of fold change vs. statistical consistence. The figure demonstrates the consistency and uniformity of differences between genes that are identified as over-expressed within HUBEC versus HUVEC.

FIG. 7. Comparison of gene expression between HUBEC and HUVEC. Color representation of the 65 transcripts that are most highly over-expressed within HUBEC (red indicator, upper left) versus HUVEC (red indicator, lower right), demonstrating consistency between biological replicates.

FIGS. 8A-8C

Noncontact culture with HUBECs increases total cells, CD34⁺ cells, and severe combined immunodeficient-repopulating cells (SRCs) compared with cytokines alone. (A): Total cell expansion is shown comparing input cord blood (CB) CD34⁺ cells versus day 14 TSF-cultured progeny versus noncontact HUBEC culture supplemented with TSF. (B): CD34⁺ cell expansion is shown demonstrating a significant increase in CD34⁺ cells following HUBEC culture compared with TSF alone at day 14. (C): SRC activity of day 0 CB CD34⁺ cells versus the progeny of CB CD34⁺ cells following 14-day culture with TSF alone versus the progeny of noncontact HUBEC-culture plus TSF at day 14. Human CD45⁺ cell engraftment was significantly higher in the nonobese diabetic severe combined immunodeficient (NOD/SCID) mice transplanted with the progeny of noncontact HUBEC cultures compared with either input or the progeny of TSF cultures. Abbreviations: HUBEC, human brain endothelial cell; SRC, severe combined immunodeficient-repopulating cell; TSF, thrombopoietin, stem cell factor, and Flt-3 ligand.

FIG. 9

Volcano plot of the nonconvergent nature of transcripts identified within human brain endothelial cells (HUBECs) and human umbilical vein endothelial cells (HUVECs). The average fold change of gene expression was calculated by comparing HUBECs (n=5) to HUVECs (n=4) in replicated experiments. Statistical significance was estimated by analysis of variance models. For each gene, the t score was plotted against the average fold change. Using stringent statistical cutoff values (gray line), 32 upregulated genes (red) and 33 down-regulated (green) were identified as differentially expressed. The minimum fold change of these selected genes was 2, and the Bonferroni corrected p value is <0.01.

FIG. 10

Expression pattern of the top 65 differentially expressed genes within HUBECs versus HUVECs. Each column represents one independent experiment and each row represents a distinct gene. The relative expression ratio between HUBECs versus HUVECs is represented by color (red, higher, green, lower, black, no change). Abbreviations: HUBEC, human brain endothelial cell; HUVEC, human umbilical vein endothelial cell.

FIGS. 11A and 11B

Adrenomedullin supports an increase in CD34⁺ progenitor cell expansion in short-term culture. Primary human cord blood CD34⁺cells (2.5×10⁴) were placed in culture with thrombopoietin, stem cell factor, and Flt-3 ligand with and without 50-100 ng/ml IGFBP2, IGFBP3, follistatin, or adrenomedullin for 7 days. (A): The addition of IGFBP2. IGFBP3, or follistatin had no effect on total progenitor cell expansion, whereas adrenomedullin caused a significant increase in total cells (p=0.001). (B): The addition of adrenomedullin also caused a significant increase in the number of CD34⁺ progenitor cells over time (p=0.002). Abbreviations: ADM, adrenomedullin; Foll, follistatin; IGFBP, insulin-like growth factor binding protein; TSF, thrombopoietin, stem cell factor, and Flt-3 ligand.

FIGS. 12A-12D

Hematopoietic activity of human brain endothelial cell (HUBEC)-secreted factors on primitive human CB CD34^{*}CD38⁻lin⁻ progenitors. FACS-sorted human CB CD34+CD38⁻lin⁻ cells (n=5 cells per well) were sorted into individual Terasaki culture wells with stem cell factor (SCF), Flt-3 ligand, or TSF with and without 50-100 ng/ml recombinant IGFBP2, IGFBP3, follistatin, or adrenomedullin. The bar graphs indicate the mean total cell expansion under each condition at day 7. As shown, IGFBP2 failed to induce significant proliferation of human progenitors (A), whereas 50 ng/ml IGFBP3 appeared to have an additive effect with Flt-3 ligand (B) (p=0.01). Follistatin had no effect on progenitor cell proliferation (C), whereas adrenomedullin demonstrated a dose-responsive additive effect on CD34⁺CD38⁻lin⁻ progenitor cell proliferation when combined with both SCF (p=0.01) and Flt-3 ligand (p=0.003) (D). Abbreviations: SCF, stem cell factor; TSF, thrombopoietin, stem cell factor, and Flt-3 ligand.

FIGS. 13A-13D

HUBEC culture supports the recovery of irradiated human hematopoietic progenitor cells. Primary human BM CD34⁺ cells were irradiated in vitro with 400 cGy and placed in culture with TSF alone, HUBEC contact culture, or HUBEC noncontact (transwell; TW) culture. The mean recovery of total cells (A) and CD34+CD38⁻ cells (B) is shown at the top and demonstrates significantly improved recovery of both populations via coculture with HUBECs under contact and noncontact conditions as compared with TSF alone. Similarly, the recovery of total cells (C) and CD34⁺CD38⁻ cells (D) within 400 cGy-irradiated CB CD34⁺ cells was also significantly greater after both contact and noncontact HUBEC culture as compared with TSF alone. The mean number of cells in the identified condition is significantly different from that in the TSF culture group.

FIGS. 14A and 14B

HUBEC coculture maintains a higher percentage of CD34⁺CD38⁻ cells after radiation injury than TSF alone. The 400 cGy-irradiated BM and CB CD34⁺ cells were placed in 10-day cultures and analyzed by flow cytometry to determine phenotype changes. A representative analysis of day 0 400 cGy-irradiated BM CD34⁺ cells is shown (Ai), along with analysis of the day 10 progeny of TSF culture, revealing a nearly complete loss of CD34⁺CD38⁻ cells (Ali). In contrast, HUBEC contact (Aiii) and HUBEC noncontact (Aiv) cultures maintained a population of CD34⁺CD38⁻ cells at day 10. Similar maintenance of CB CD34⁺CD38⁻ cells after 400 cGy was also observed during HUBEC contact and noncontact cultures (B). The percentages of cells in each quadrant are shown in the upper right of each figure. FITC indicates fluorescein isothiocyanate.

FIGS. 15A and 15B

HUBEC contact and noncontact cultures promote the recovery of CFCs compared with TSF alone. Day 0 normal and 400 cGy-irradiated CD34⁺ cells and the day 10 progeny of 400 cGy-irradiated CD34⁺ cells after culture with TSF alone, HUBEC contact, and HUBEC noncontact culture were analyzed for CFC content after 14 days. The 400-cGy exposure caused a significant reduction in human CFC content at day 0. The progeny of 400 cGy-irradiated BM CD34⁺ cells (A) and CB CD34⁺ cells (B) after HUBEC contact and noncontact cultures contained significantly more CFU-total as compared with the progeny of 400 cGy-irradiated CD34⁺ cells cultured with TSF alone. The mean number of cells in the identified condition is significantly different from that in the TSF culture group. D indicates day.

FIGS. 16A and 16B

HUBEC coculture decreases hematopoietic progenitor cell death after radiation injury. Primary human BM CD34⁺ cells (>95% purity) were irradiated with 400 cGy and subsequently placed in culture with TSF alone or HUBEC contact and noncontact cultures supplemented with TSF. Flow cytometric analysis was performed to measure the percentage of apoptotic and necrotic cells in each condition over time. Day 0 nonirradiated BM CD34⁺ cells and 400 cGy-irradiated BM CD34⁺ cells were analyzed as controls. A, Analysis of the entire population demonstrated that 400 cGy caused a significant increase in both apoptotic and necrotic cells by 6 hours after exposure (P<0.05 for each comparison). By days 3 and 10, a modest but significant decrease in the percentage of apoptotic and necrotic cells was observed within HUBEC contact and noncontact cultures as compared with TSF alone (P<0.05). B, Analysis of the CD34⁺ progenitor cell subset over time demonstrated a more significant reduction in cell death within the HUBEC contact and noncontact cultures as compared with TSF alone (P<0.05 for each comparison), thus suggesting a differential effect of endothelial cell culture on progenitor cell repair after radiation injury. The mean percentage of cells in the identified condition is significantly different from that in the TSF culture group.

FIG. 17

HUBEC coculture supports the recovery of human BM long-term repopulating cells after radiation injury. NOD/SCID mice underwent transplantation with 0.75 to 1.5×10⁶ nonirradiated or 400 cGy-irradiated BM CD34⁺ cells per mouse or the progeny of 400 cGy-irradiated BM CD34⁺ cells after culture with TSF alone or HUBEC contact (open circles) and noncontact (filled circles) cultures. The dose of 400 cGy caused a marked reduction in day 0 SRC content, and culture of 400 cGy-irradiated BM CD34⁺ cells with TSF alone was associated with a complete loss of SRC over time. Conversely, noncontact culture with HUBECs maintained SRC content, thus indicating that soluble factors produced by HUBECs contributed CO HSC repair.

FIGS. 18A(i-iv) and 18B(i-iv)

Noncontact culture of 400 cGy-irradiated BM CD34⁺ cells with HUBECs maintains cells with multilineage differentiative capacity. A, Representative NOD/SCID BM analysis is shown from mice injected with 1.5×10⁶ nonirradiated BM CD34⁺ cells (Ai), 400 cGy-irradiated day 0 BM CD34′ cells (Ali), or the progeny of the identical dose of 400 cGy-irradiated BM CD34′ cells after a 10-day culture with TSF alone (Aiii) or HUBEC noncontact culture (Aiv). B, Multiparameter flow cytometric analysis was performed on engrafted human cells in representative mice. Isotype control staining is shown (Bi), and CD34⁺ progenitor cells were demonstrated (Bii), as were CD19⁺ B cells (Biii) and CD13⁺ myeloid cells (Biv) in transplanted mice at 8 weeks. The relative proportion of B cells in the transplanted mice was increased as compared with myeloid cells; this indicates that lymphoid recovery may have occurred more rapidly after radiation injury. FITC indicates fluorescein isothiocyanate; PerCP, peridinin-chlorophyll protein complex.

### DETAILED DESCRIPTION OF THE INVENTION

Stem cells contained within the hematopoietic compartment represent the most well defined and clinically utilized adult stem cells. Hematopoietic stem cells (HSC) have the unique capacity to undergo self-renewal in vivo throughout the life of an individual while also providing the complete repertoire of mature hematopoietic and immune cells. Currently, transplantation of human HSC from adult bone marrow (BM), mobilized peripheral blood (PB) and umbilical cord blood (CB) is successfully applied in the curative treatment of both malignant and non-malignant diseases. More recently, the potential contribution of transplanted HSC toward immune tolerance induction, vascular remodeling, and in vivo tissue regeneration has been demonstrated. Since HSC comprise <0.01% of the CD34+ population within the human hematopoietic compartment in adults, numerous studies have attempted to expand HSC numbers in vitro with a goal of generating larger numbers of transplantable repopulating cells. Concordantly, strategies have been applied to identify the heretofore unknown growth factors that stimulate HSC self-renewal in vivo. Unfortunately, currently available cytokines have not been shown to support adult human HSC expansion in vitro or in vivo.

The present invention is based on a strategy to identify novel HSC growth factors. It involves examination of candidate niches wherein HSC are known to reside physiologically. During embryogenesis, development of the primitive hematopoietic and vascular system development are interdependent, such that VEGFR2-knockout mice fail to develop either blood islands or vascular development at day 8. Gene marking studies have also suggested a common precursor cell, the hemangioblast, which appears to give rise to both hematopoietic stem cells and endothelial precursor cells (EPC). Endothelial cells (EC) from the aorto-gonado-mesonephros region have been shown to support murine HSC growth ex vivo and EC from adult bone marrow support the in vitro maintenance of erythroid, myeloid, and megakaryocytic progenitor cells. Anatomically, HSC embed within the endothelial cell-lined intimal layer of the aorta at day 35 of human embryogenesis and reside in contact with endothelial cells in the adult bone marrow. Therefore, vascular endothelial cells appear to be a logical source of potentially novel HSC growth factors.

More recently, it has also been shown that osteoblasts may represent a contributory nich for hematopoietic stem cells in vivo. In addition, stromal cell lines derived from murine fetal liver have recently been demonstrated to support the maintenance of murine and human HSC in vitro. Since such stromal cell lines are heterogeneous, it remains unclear which cell type accounts for the observed hematopoietic effects. Importantly, studies of stromal cell lines have also demonstrated the critical requirement for cell-to-cell contact between the stromal cells and HSC for HSC maintenance to occur.

Experimentally, co-culture with microvascular endothelial cells derived from porcine brain (PM VEC) were demonstrated by Davis T et al. to support the maintenance of human CD34+ and CD34+CD38− cells during short term culture. It was subsequently demonstrated that this porcine brain endothelial cell line induced primitive human CD34+CD38− HSC to proliferate at a high rate, augmented the retroviral mediated genetic modification of human HSC, and was capable of inducing the complete functional repair and expansion of murine HSC following exposure to lethal doses of ionizing radiation. In collaboration with Brandt et al, it was demonstrated that human bone marrow cells expanded in co-culture with porcine brain EC were capable of repopulating SCID-Hu femurs and providing complete hematopoietic reconstitution in baboons following lethal irradiation. In order to determine whether this brain endothelial cell-derived hematopoietic activity was conserved across species, several primary human brain endothelial cell lines (HUBEC) were subsequently developed and their capacity to expand human HSC in vitro was tested. Primary HUBEC, which are homogeneous (>95% Von Willebrand Factor positive), support the 4-fold expansion of human BM and CB HSC in 7 day cultures as measured by rigorous limiting dilution analysis of NOD/SCID repopulating cells (SRC). Conversely, non-brain EC failed to support the maintenance or expansion of human BM CD34+CD38− cells. Extended cultures (14 day) of purified CD34+CD38− HSC with HUBEC have demonstrated approximately an additional log increase in human SRC, suggesting ongoing self-renewal of HSC over time. Most importantly, in contrast to previously described murine and human stromal cell lines, when cell-to-cell contact between human HSC and HUBEC was prevented, persistent and enhanced expansion of BM and CB SRC was observed, indicating that soluble factors elaborated by HUBEC were responsible for this stem cell expansive effect.

Example 2 below includes a description of the application of high throughput genomics analysis of multiple biologic replicates of primary HUBEC with a goal to identify the candidate secreted proteins responsible for human HSC expansion. Via subtraction of the HUBEC transcriptional profile versus non-brain EC (Human Umbilical Vein Endothelial Cells, HUBEC), many annotated and non-annotated transcripts have been identified that are markedly and specifically overexpressed by HUBEC. A high percentage of these transcripts are involved in cell-to-cell communication processes, anti-apoptosis, and morphogenesis. Soluble and secreted proteins with candidate hormonal activity are also significantly over-represented in the HUBEC molecular profile. This profile represents a signature of a human HSC niche and a template for further biologic study of particular proteins that have a likelihood of participation in HSC self renewal.

It will be appreciated that the identification and development of novel growth factors that induce HSC expansion has therapeutic application, for example, in cord blood transplantation, immune tolerance induction, tissue regeneration, vascular remodeling and genetic modification.

The described list of novel endothelial-derived transcripts and soluble proteins (see Example 2) represent unique candidates that have been identified to be associated with the in vitro expansion of HSC. These molecules can be reduced to clinical practice through the following studies:

i) in vitro activity assays of individual proteins against human BM, CB CD34+ cells, ii) in vivo repopulating cell assay of human CD34+ cells following in vitro stimulation with individual proteins identified herein, and iii) in vivo administration of individual proteins to irradiated mice to assess in vivo stimulation of hematopoietic stem cell compartment.

Certain aspects of the invention can be described in greater detail in the non-limiting Examples that follow. (See also Chute et al, Stem Cells 24:1315-1327 (2006) (online publication Dec. 22, 2005) wherein it is reported that extended noncontact HUBEC cultures supported an eight-fold increase in SRCs when combined with thrombopoietin, stem cell factor, and Flt-3 ligand compared with input CD34⁺ cells or cytokines alone. Gene expression analysis of HUBEC biological replicates identified 65 differentially expressed, nonredundant transcripts without annotated hematopoietic activity. Gene ontology studies of the HUBEC transcriptome revealed a high concentration of genes encoding extracellular proteins with cell-cell signaling function. Functional analyses demonstrated that adrenomedullin, a vasodilatory hormone, synergized with stem cell factor and Flt-3 ligand to induce the proliferation of primitive human CD34⁺CD38⁻lin⁻ cells and promoted the expansion of CD34⁺ progenitors in culture.

### Example 1

### Experimental Details

Isolation of Human BM CD34⁺CD38⁻ Cells Prior to Ex Vivo Culture

Human BM CD 34⁺ cells were acquired from Biowhittaker (Gaithersburg, Md.). CD34⁺ cells (>95% purity) were thawed and placed in complete culture medium containing IMDM (Invitrogen, Carlsbad, Calif.), 10% FBS (Hyclone, Logan, Utah), 100 U/mL penicillin and 100 μg/mL streptomycin (1% pcn/strp) at 37° C. Cells were then pelleted and resuspended in phosphate buffered saline (PBS), counted and stained with anti-CD34-fluorescein isothiocyanate (F′T′C) and anti-CD38-phycoerythrin (PE) (Becton Dickinson, San Jose, Calif.). After 30 minutes on ice, the cells were washed twice and resuspended in PBS containing 10% heat-inactivated FBS, 1% pcn/strp. Samples were the analyzed and sorted using a MoFlo cell sorter (DakoCytomation, Carpinteria, Calif.) to isolate CD34⁺CD38⁻ and CD34⁺CD38⁺ subsets. The CD34⁺CD38⁻ sort gate was set to collect only those events falling in the lowest 2% of PE fluorescence within the total CD34⁺ population, to ensure acquisition of highly purified CD34⁺CD38⁻ cells.

### Ex Vivo Culture of CD34⁺, CD34⁺CD38⁻, CD34⁺CD38⁺ Cells

HUBEC monolayers were established in culture as previously described (Chute et al, Blood 100:4433-4439 (2002)). Briefly, 1×10⁵ HUBEC cells were cultured on gelatin-coated 6-well plates (Costar, Cambridge, Mass.) in complete endothelial cell culture medium (5 ml/well) containing M199 (Invitrogen), 10% FBS, 100 μg/mL L-glutamine (Invitrogen), 50 μg/mL heparin, 60 μg/mL endothelial cell growth supplement (Sigma, St. Louis, Mo.), and pcn/strp at 37° C. in 5% CO₂ atmosphere. After 48 hours, HUBEC were washed twice with PBS, and the media was replaced with BM expansion medium (5 ml/well) containing IMDM, 10% FBS, pcn/strp, 2 ng/ml granulocyte macrophage-colony stimulating factor (GM-CSF), 5 ng/ml interleukin 3 (IL-3), 5 ng/ml IL-6, 120 ng/mL stem cell factor (SCF), and 50 ng/ml flt-3 ligand (GM36SF; R&D Systems, Minneapolis, Minn.). BM CD34⁺CD38⁻ sorted cells were then added at 1×10³-4×10⁴ cells/well and maintained at 37° C. in 5% CO₂ atmosphere. After 7 days of HUBEC co-culture, non-adherent cells were harvested by washing the so monolayers gently with warm IMDM. For comparison, BM CD34⁺CD38⁺ sorted cells were cultured at 1×10⁵ cells per well under identical conditions. In order to determine the effect of HUBEC-soluble activity, FACS-sorted BM CD34⁺CD38⁻ cells were also cultured with HUBEC separated by a 0.4 micron transwell insert (CoStar, Cambridge, Mass.). The effect of HUBEC co-culture of BM CD34⁺CD38⁻ cells supplemented with 20 ng/mL thrombopoietin, 120 ng/mL SCF, and 20 ng/mL flt-3 ligand (TSF) was also measured in 7 day cultures. As controls, FACS-sorted BM CD34⁺CD38⁻ were also plated in liquid suspension cultures with the GM36SF and TSF. Hemacytometer counts were performed to determine cellular expansion. Multiple donor BM CD34⁺ cells were combined prior to FACS-sorting of BM CD34⁺CD38⁻ cells so that identical cell populations were utilized for initiation of all comparative experiments.

Immunophenotype Analysis and Colony Forming Assays of BM CD34⁺CD38⁻ cells Pre- and Post-Culture

Freshly sorted BM CD34⁺CD38⁻ cells and BM CD34⁺CD38⁺ cells cultured either with HUBEC monolayers or liquid suspension culture were analyzed for phenotypic changes at day 4 and day 7 of culture. The cells were stained with CD34-FITC and CD38-PE for 30 minutes on ice and compared with appropriate isotype control antibody staining. Samples were analyzed using a MoFlo cell sorter with Summit software (DakoCytomation). Colony forming assays of day 0 BM CD34⁺CD38⁻ cells and the progeny of BM CD34⁺CD38⁻ cells cultured with HUBEC+GM36SF or GM36SF alone were performed as previously described (Chute et al, Blood 100:4433-4439 (2002)). Cells (5-50×10²) were cultured in 35-mm culture dishes (Miles Laboratories, Naperville, Ill.) in media consisting of 1 mL of IMDM, 1% methylcellulose, 30% FBS, 5 U/mL erythropoietin, 2 ng/mL GM-CSF, 10 ng/mL IL-3, and 120 ng/mL SCF. At day 14, triplicate cultures were evaluated to determine the number of colonies (>50 cells) per dish.

### NOD/SCID Transplantation Studies

NOD/SCID mice (Schulz et al, J. Immunol. 154:180-191 (1995)) were transplanted with either FACS-sorted BM CD34⁺CD38⁻ cells or the progeny of BM CD34⁺CD38⁻ cells cultured with HUBEC monolayers supplemented with GM36SF over a range of doses. Cells were transplanted via tail vein injection after irradiating NOD/SCID mice with 300 cGy using a linear accelerator source as previously described (Chute et al, Blood 100:4433-4439 (2002)). Mice transplanted with day 0 BM CD34⁺CD38⁻ cells were co-transplanted with 2×10⁵ CD34⁻ accessory cells to facilitate engraftment as previously described (Bhatia et al, Proc. Natl. Acad. Sci. USA 94:5320-5325 (1997), Bonnet et al, Bone Marrow Transpl. 23:203-209 (1999)). Mice transplanted with the progeny of BM CD34⁺CD38⁻ cells following 7 days of HUBEC culture received no CD34⁻ accessory cells or exogenous cytokines to facilitate engraftment. Additional groups of mice were transplanted with FACS-sorted subsets of HUBEC progeny at day 7. All mice in each group were sacrificed at week 8 and marrow samples were obtained by flushing their femurs with IMDM at 4° C. Red cells were lysed using red cell lysis buffer (Sigma) and flow cytometric analysis of human hematopoietic engraftment was performed as previously described using commercially available monoclonal antibodies against human leukocyte differentiation antigens to identify engrafted human leukocytes and discriminate their hematopoietic lineages (Chute et al, Blood 100:4433-4439 (2002), Trischmann et al, J. Hematother. 2:305-313 (1993)).

### FACS Sorting and Transplantation of HUBEC-Cultured Subpopulations

In order to define the phenotype of amplified SRC following HUBEC culture, BM CD34⁺CD38⁻ cells which had been cultured×7 days with HUBEC+GM36SF were collected, washed in IMDM, centrifuged, and then stained with CD34 FITC and CD38-PE. An aliquot of cells was also stained with IgG-FITC and IgG-PE antibodies for control staining. After 30 minutes on ice, the cells were resuspended in PBS with 10% FBS/1% pcn/strep, centrifuged, and resuspended in IMDM+10% FBS. Samples were then analyzed and sorted using a MoFlo cell sorter. Since HUBEC-cultured cells showed predominance of either the CD34⁺CD38⁻ or CD34⁻CD38⁻ phenotype, we collected the CD34⁺CD38⁻ and the CD34⁻CD38⁻ populations post-culture. The sorted populations were centrifuged, resuspended in PBS and transplanted via tail vein injection into NOD/SCID mice for measurement of repopulating capacity. Each mouse was transplanted with the cultured product of 40,000 FACS-sorted BM CD34⁺CD38⁻ cells per condition.

### Statistical Analysis and SRC Frequency Measurements

For purposes of the limiting dilution analysis, a transplanted mouse was scored as positively engrafted if ≧0.1% of the marrow cells expressed human-CD45 via high resolution FACS analysis. This criteria is consistent with previously published criteria for human cell repopulation in NOD/SCID mice (Glimm et al, Blood 94:2161-2168 (1999), Dorrell et al, Blood 95:102-110 (2000)) SRC frequency in each cell source was calculated using the maximum likelihood estimator as described previously by Taswell, J. Immunol. 126:1614-1619 (1981)) for the single hit Poisson model (Wang et al, Blood 89:3919-3924 (1997), Ueda et al, J. Clin. Invest. 105:1013-1021 (2000)). The provides a measure of the legitimacy of using pooled data and of the validity of applying the single hit model (Wang et al, Blood 89:3919-3924 (1997). Confidence intervals were calculated for the frequencies using the profile likelihood method, and the likelihood ratio test was used to confirm the fit of the model. As a confirmation of the maximum likelihood estimator, a minimum estimator was applied to the pooled data.

### Results

### HUBEC Co-Culture Increases Total Cells, CD34⁺, and CD34⁺CD38⁻ Cells

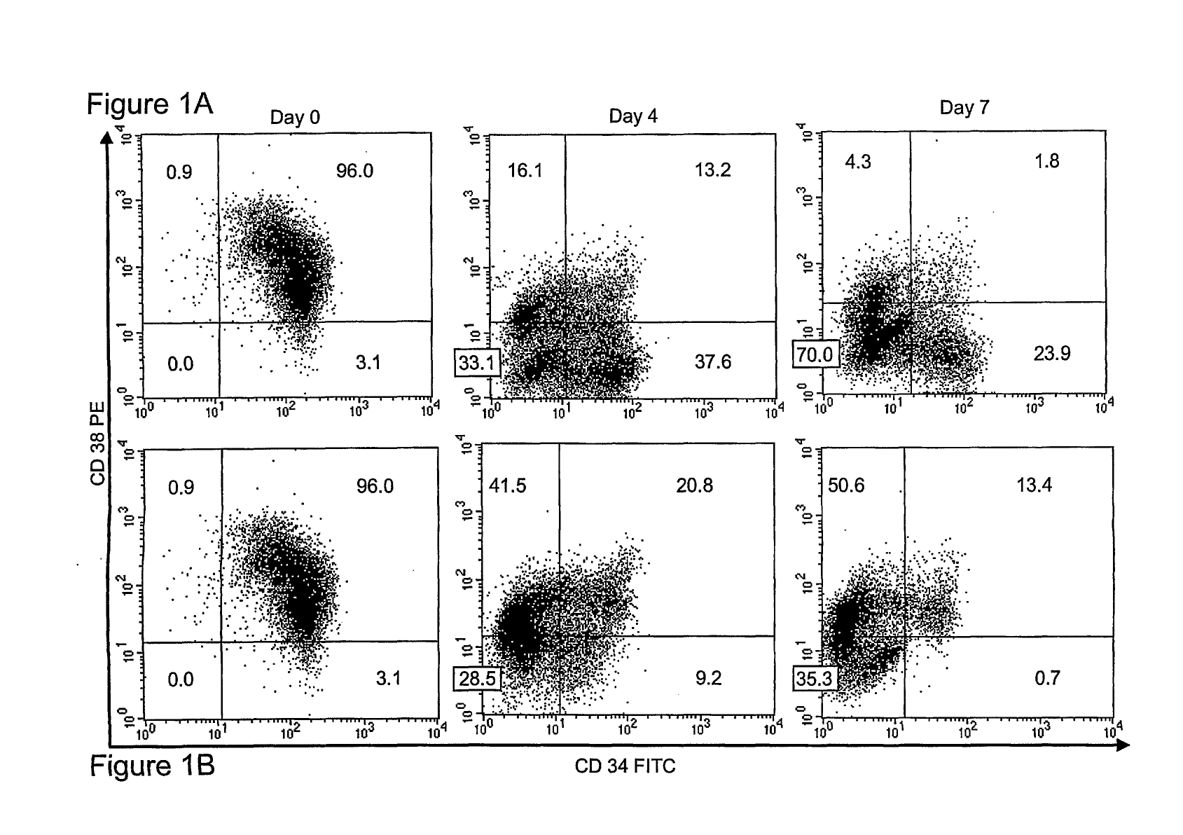

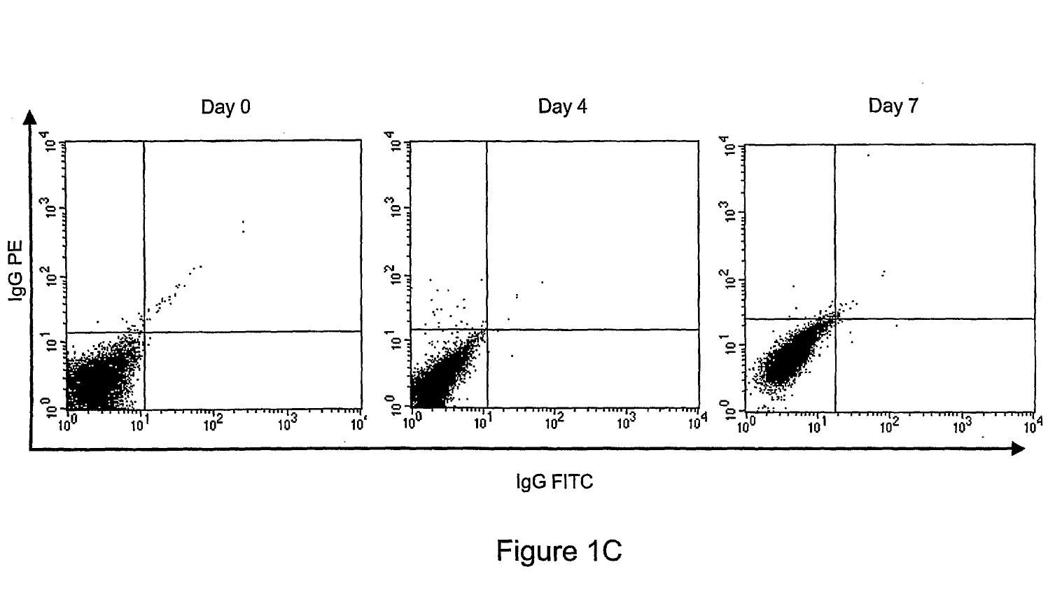

First examined was the effect of HUBEC culture+GM36SF and GM36SF alone on the expansion of human BM CD34⁺ cells (n=3). FIG. 1A shows a representative BM CD34⁺ cell sample from day 0 through day 7 of HUBEC culture. Input cells were 99.1%±0.1 CD34 positive, with 96%±0.1 of these cells being CD34⁺CD38⁺ and 3.2±0.1% being CD34⁺CD38⁻, defined as CD34⁺ cells showing PE fluorescence less than the IgG PE isotype control. By day 4 of HUBEC culture+GM36SF, 36.7%±0.7 of the cells became CD34⁺CD38⁻, and by day 7, 22.6%±0.1 of the cultured cells were CD34⁺CD38⁻. At day 7 of HUBEC culture+GM36SF, a mean 16-fold increase in total cells, a 3.9-fold increase in CD34⁺ cells, and a 115-fold increase in CD34⁺CD38⁻ cells were calculated (Table 1). For comparison, liquid suspension cultures of BM CD34⁺ cells with GM36SF alone caused a mean 19.3-fold increase in total cells and a 2.6-fold increase in CD34⁺ cells at day 7, but the CD34⁺CD38⁻ population was nearly completely lost during 7 day culture (9.8%±0.3 CD34⁺CD38⁻ cells at day 4, <1% at day 7). FIG. 1B shows representative phenotypic changes within BM CD34⁺ cells during culture with GM36SF alone over 7 days.

TABLE 1
Expansion of purified BM CD34⁺ subsets following co-culture with HUBEC monolayers

| | Day 0 (Cell | | | | |
|---|---|---|---|---|---|
| | counts × | | | | |
| | 10⁵) | | | | |
| Culture | Cell | | Day 7 (Cell counts × 10⁵) | | |
| condition | subset | Cell Ct | Total | No. of CD34⁺ | No. of CD34⁺CD38⁻ |
| HUBEC (contact) | 34⁺ | 1.0 | 16.2 ± 0.3 | 3.9 ± 0.1 | 3.7 ± 0.1 |
| | | | (16-fold) | (3.9-fold) | (115-fold) |
| GM36SF | 34⁺38⁺ | 1.0 | 18.3 ± 0.1 | 2.6 ± 0.1 | 2.4 ± 0.1 |
| | | | (18-fold) | (2.6-fold) | (2.4-fold) |
| | 34⁺38⁻ | 0.4 | 2.7 ± 0.7 | 1.8 ± 0.5 | 1.8 ± 0.5 |
| | | | (6.7-fold) | (4.5-fold) | (4.4-fold) |
| HUBEC (non-contact) + | 34⁺38⁻ | 0.4 | 6.7 ± 0.1 | 3.4 ± 0.5 | 2.5 ± 0.1 |
| GM36SF | | | (16.7-fold) | (8.6-fold) | (6.4-fold) |
| HUBEC (contact) + | 34⁺38⁻ | 0.4 | 5.8 ± 0.5 | 5.3 ± 0.4 | 5.2 ± 0.5 |
| | | | (14.6-fold) | (13.4-fold) | (13.1-fold) |
| GM36SF | 34⁺38⁻ | 0.4 | 14.4 ± 0.7 | 3.7 ± 0.2 | 0.3 ± 0.1 |
| | | | (35.8-fold) | (9.2-fold) | (—) |
| TSF | 34⁺38⁻ | 0.4 | 1.4 ± 0.2 | 1.0 ± 0.1 | 0.1 ± 0.1 |
| | | | (3.5-fold) | (2.4-fold) | (—) |

Human BM CD34⁺ cells (99.1% ± 0.1 CD34⁺, 3.2% ± 0.1 CD34⁺CD38⁻96.0% ± 0.1 CD34⁺CD38⁺) were cultured with HUBEC supplemented with GM36SF (n = 3).
FACS-sorted BM CD34⁺CD38⁻ cells (n = 8) and BM CD34⁺CD38⁺ cells (n = 6) were placed in contact or non-contact cultures with HUBEC monolayers and cytokine supplements as indicated above.
For comparison, BM CD34⁺CD38⁻ cells were cultured in liquid suspension with either GM36SF or TSF (n = 3).

### Characterization of the CD34⁺CD38⁻ Cells Produced During HUBEC Co-Culture

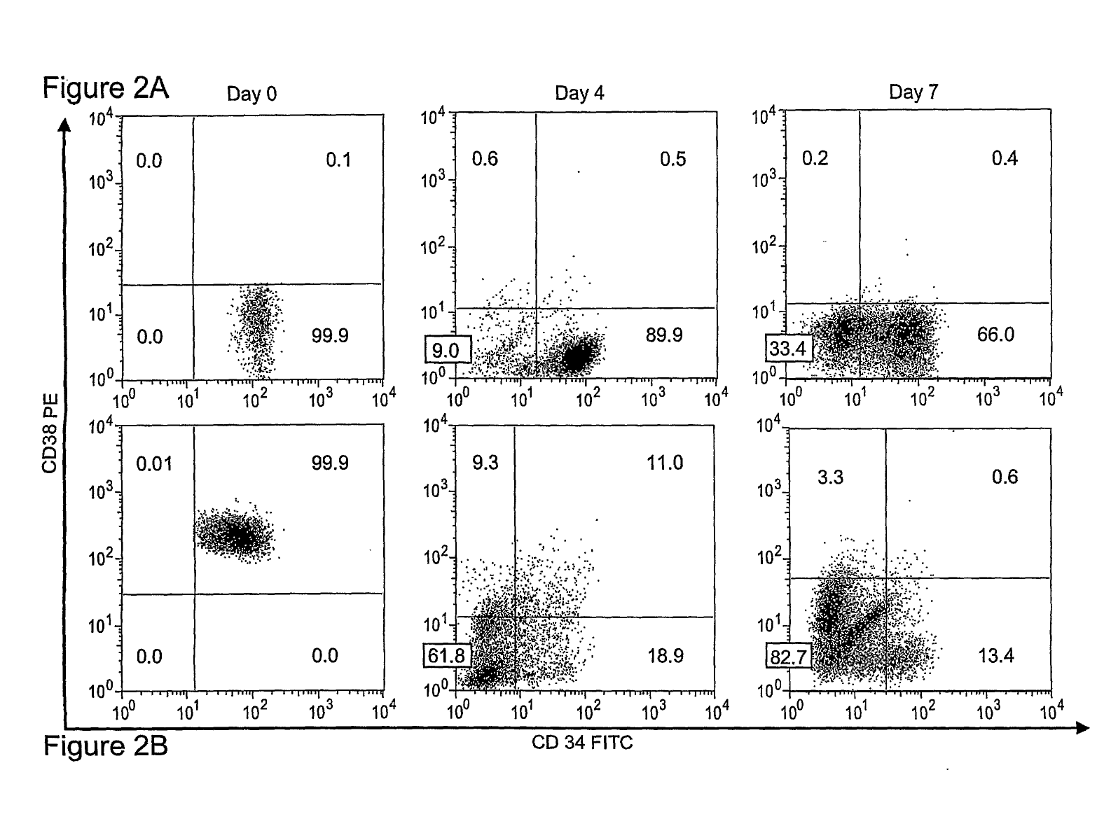

Next performed were experiments (n=8) to characterize the effect of HUBEC culture on more primitive BM CD34⁺CD38⁻ cells. As shown in FIG. 2A, the purity of day 0 FACS-sorted CD34⁺CD38⁻ populations were >98% in all experiments. At day 4 and 7 of HUBEC culture+GM36SF, 93.0%±1.4 and 67.6%±10.7 of the cultured cells remained CD34⁺CD38⁻, respectively, concomitant with a mean 6.7-fold increase in total cells at day 7. This translated into a mean 4.4-fold increase in CD34⁺CD38⁻ cells. The majority of the remaining cells at day 7 were CD34⁻CD38⁻ (32%±10.6). FACS analysis of BM CD34⁺CD38⁻ cells from a representative experiment at day 0, day 4, and day 7 of HUBEC culture is shown in FIG. 2A. Table 1 summarizes the expansion of BM CD34⁺CD38⁻ cells during HUBEC culture+GM36SF from 8 experiments.

In order to determine whether down-modulation of CD38 antigen expression on CD34⁺CD38⁺ cells contributed to the increase in CD34⁺CD38⁻ cells observed during HUBEC culture of unsorted BM CD34⁺ cells, the expansion of FACS-sorted BM CD34⁺CD38⁺ cells during HUBEC culture+GM36SF (n=6 experiments) was examined. FIG. 2B shows a representative phenotype of input BM CD34⁺CD38⁺ cells and the progeny of BM CD34⁺CD38⁺ cells during 7 day HUBEC culture. By day 4, only 29.7%±1.0 of the cells remained CD34⁺and 20.6%±1.1 were CD34⁺CD38⁻. By day 7, more than 85% of the input CD34⁺CD38⁺ cells became phenotypically CD34⁻, but 13.3%±1.2 of the day 7 population demonstrated a CD34^{dim}CD38⁻ phenotype, while the total cell number increased by a mean 18-fold. Therefore, from an input of 1×10⁵ BM CD34⁺CD38⁺ cells, HUBEC culture yielded 2.4×10⁵ “CD34⁺CD38⁻” cells or a 240% increase in phenotypic “CD34⁺CD38⁻.” cells compared to input.

Consistent with the observation of Dorrell et al. regarding ex vivo culture of CB (Dorrell et al, Blood 95:102-110 (2000)), the phenotypic changes which purified BM CD34⁺CD38⁺ and CD34⁺CD38⁻ cells undergo during HUBEC co-culture can be extrapolated to the ex vivo expansion of unsorted BM CD34⁺ cells on HUBEC monolayers. A typical BM CD34⁺ sample of 1×10⁶ cells contains 9.5×10⁵ (95%) CD34⁺ cells, 9×10⁵ (90%) CD34⁺CD38⁺ cells, and 5×10⁴ (5%) CD34⁺CD38⁻ cells. Given the yields observed with purified BM cell subsets, the CD34⁺CD38⁺ fraction should produce 2.2×10⁶ “CD34⁺CD38⁻” cells and the input CD34⁺CD38⁻ cells should contribute 2.0×10⁵ CD34⁺CD38⁻ cells. Therefore, 91.7% of the CD34⁺CD38⁻ cells recovered from CD34⁺ cells cultured with HUBEC at day 7 would be predicted to derive from committed CD34⁺CD38⁺ cells which contain no repopulating capacity.

### Effect of Non-Contact HUBEC Culture and Alternative Cytokine Combinations on BM CD34⁺CD38⁻ Cell Expansion

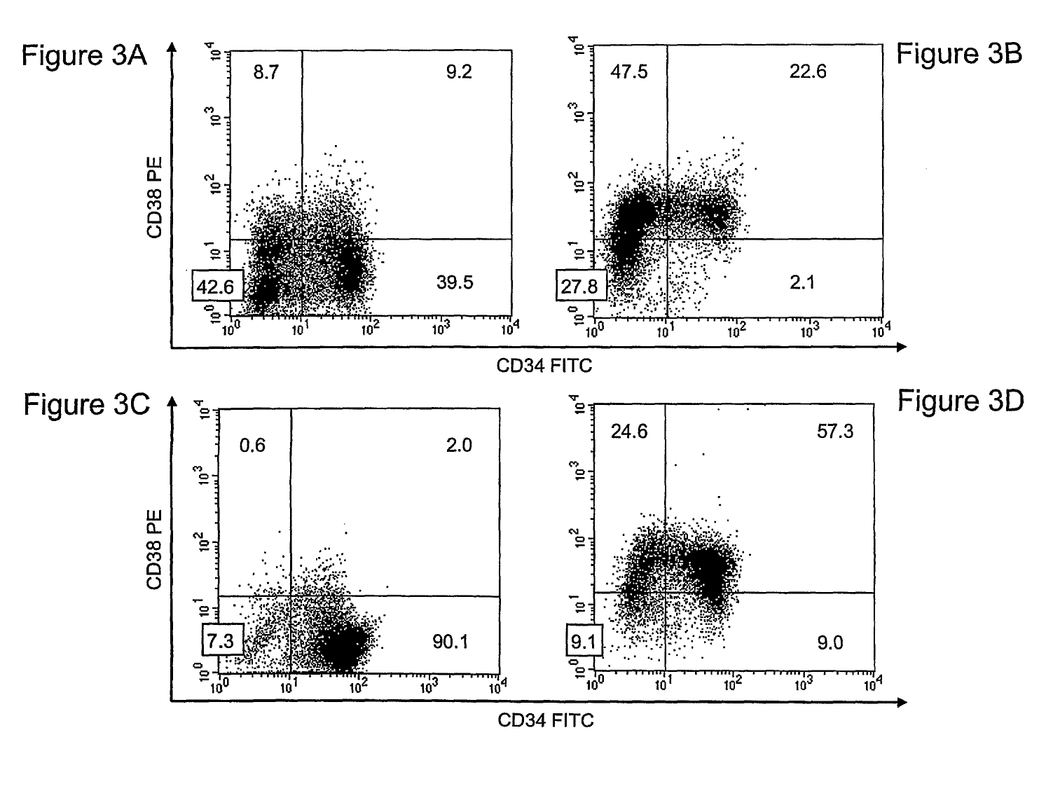

FACS-sorted BM CD34⁺CD38⁻ cells were cultured with HUBEC separated by transwell inserts to ascertain the importance of cell-to-cell contact on HUBEC-mediated hematopoietic expansion. Interestingly, non-contact HUBEC cultures+GM36SF supported a greater increase in total cells (mean 16.7-fold; n=3) and CD34⁺CD38⁻ cells (mean 6.4-fold) compared to HUBEC contact cultures, with 38.2%±1.3 of the day 7 population expressing the CD34⁺CD38⁻ phenotype. FIG. 3A shows a representative phenotype of BM CD34⁺CD38⁻ cells following 7 day non-contact HUBEC culture+GM36SF. In comparison studies, GM36SF alone supported a marked increase in total cells (mean 36-fold; n=3) but only 2.3%±0.2 of the population remained CD34⁺CD38⁻ at day 7, resulting in a loss of CD34⁺CD38⁻ cells compared to input. A representative phenotype of BM CD34⁺CD38⁻ cells following 7 day culture with GM36SF alone is shown in FIG. 3B. The results of these experiments are summarized in Table 1.

Since the combination of TSF has been shown to optimize the ex vivo maintenance of cord blood SRC (Piacibello et al, Blood 93:3736-3749 (1999), Tanavde et al, Exp. Hematol. 30:816-823 (2002)), the expansion of purified BM CD34⁺CD38⁻ cells during HUBEC culture+TSF was also examined. HUBEC culture+TSF supported a mean 14.6-fold (n=3) increase in total cells with 90.2%±0.1 remaining CD34⁺CD38⁻ at day 7 (FIG. 3C). This translated into a 13.1-fold increase in CD34⁺CD38⁻ cells. Conversely, liquid suspension cultures with TSF alone resulted in a mean 3.5-fold increase in total cells (n=3) with only 8.8%±1.3 of the population remaining CD34⁺CD38⁻ at day 7 (FIG. 3D). As observed with GM36SF alone, TSF alone was associated with a loss of CD34⁺CD38⁻ cells at day 7 compared to input (Table 1).

Co-Culture of BM CD34⁺CD38⁻ Cells with HUBEC Inhibits Progenitor Cell Maturation

In order to determine the effect of HUBEC culture on the differentiation of primitive BM CD34⁺CD38⁻ cells, the CFC activity was measured within day 0 BM CD34⁺CD38⁻ cells and within the progeny of BM CD34⁺CD38⁻ cells following 7 culture with HUBEC+GM36SF (n=6). As expected, day 0 BM CD34⁺CD38⁻ cells contained nearly undetectable CFC activity, reflecting a more primitive HSC-enriched population (Table 2). Following HUBEC culture+GM36SF, the progeny of BM CD34⁺CD38⁻ cells contained 12.5-fold increased CFU-GM, 4-fold increased BFU-E, and 10-fold increased CFU-total compared to input BM CD34⁺CD38⁻ cells. Conversely, the progeny of BM CD34⁺CD38⁻ cells cultured with GM36SF alone contained 70-fold increased CFU-GM, 26-fold increased BFU-E, and 54-fold increased CFU-total compared to input BM CD34⁺CD38⁻ cells. These data indicated that co-culture of BM CD34⁺CD38⁻ cells with HUBEC significantly delayed or inhibited lineage commitment of CD34⁺CD38⁻ hematopoietic stem/progenitor cells, as evidenced by approximately 6-fold lower total CFC production compared to BM CD34⁺CD38⁻ cells cultured with GM36SF alone (P=0.004; Kruskal-Wallis test).

TABLE 2
HUBEC co-culture inhibits colony forming cell differentiation
compared to cytokines alone

| | Number of Colony Forming Cells × 10³ | | | |
|---|---|---|---|---|
| Condition | CFU-GM | BFU-E | CFU-Mix | CFU-Total |
| BM | 6.3 ± 0.2 | 2.4 ± 1.3 | 1.1 ± 0.2 | 9.9 ± |
| CD34⁺CD38⁻ | | | | 2.5 |
| (Day 0) | | | | |
| Day 7 | 76.2 ± 27.0 | 8.6 ± 4.3 | 11.5 ± 2.5 | 96.4 ± |
| HUBEC + | | | | |
| GM36SF | | | | 26.3 |
| Day 7 | 411.8 ± 26.0 | 62.3 ± 28.6 | 54.8 ± 39.3 | 529.2 ± |
| GM36SF | | | | 92.6 |

The colony forming cell (CFC) activity of Day 0 FACS-sorted human BM CD34⁺CD38⁻ cells and their progeny following 7 day culture with HUBEC + GM36SF or GM36SF alone was measured (n = 6). Cells were collected and placed in methylcellulose colony forming assay cultures as described in the above. At day 14, the number of CFCs were counted in triplicate in each condition. The values presented reflect the mean number of CFCs per culture condition ± standard deviations.

HUBEC Culture Increases the SRC Frequency within BM CD34⁺CD38⁻ Cells

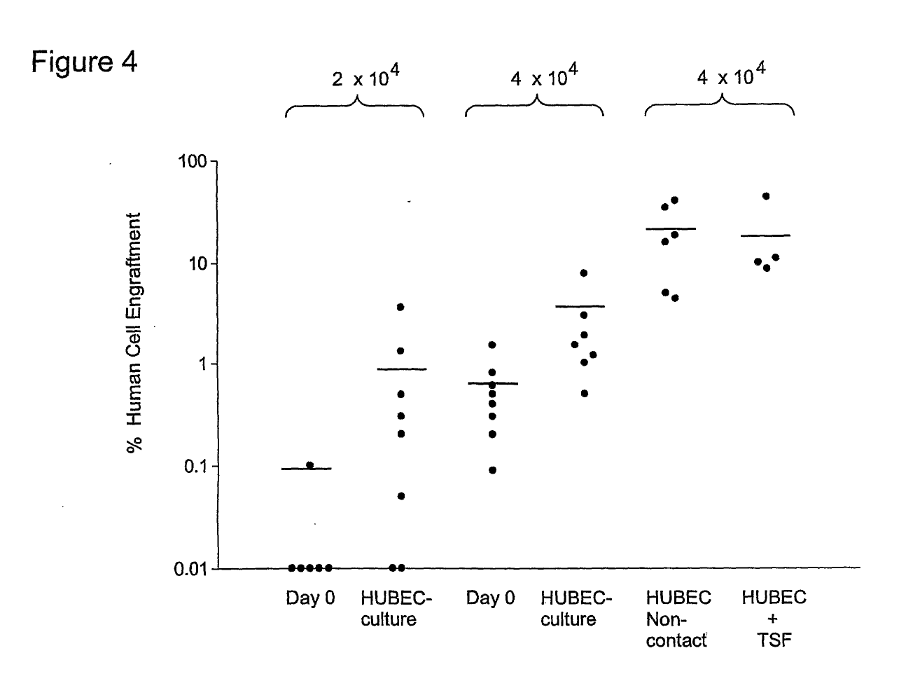

In a series of 5 experiments, NOD/SCID mice were transplanted with FACS-sorted BM CD34⁺CD38⁻ cells (n=34 mice) or the progeny of HUBEC+GM36SF cultured BM CD34⁺CD38⁻ cells (n=35 mice) over a range of doses designed to achieve non-engraftment in a fraction of mice. As negative controls, n=5 mice were transplanted with 2×10⁵ human CD34⁻ accessory cells, verifying no NOD/SOD engraftment from this population. As shown in FIG. 4, transplantation of 1×10³-1×10⁴ day 0 BM CD34⁺CD38⁻ cells resulted in no detectable human cell engraftment in NOD/SCID mice. Similarly, transplantation of the progeny of 1×10³-1×10⁴ BM CD34⁺CD38⁻ cells following HUBEC culture also resulted in no engraftment. At a dose of 2×10⁴ day 0 BM CD34⁺CD38⁻ cells, 1 of 6 mice (16.6%) showed human engraftment. Conversely, the progeny of 2×10⁴ BM CD34⁺CD38⁻ cells following HUBEC co-culture engrafted in 7 of 8 mice (88%). As shown in FIG. 4, at a dose of 4×10⁴ day 0 BM CD34⁺CD38⁻ cells, 5 of 8 mice (62.5%) showed engraftment, whereas 7 of 7 mice (100%) of mice transplanted with the progeny of 4×10⁴ BM CD34⁺CD38⁻ cells showed human cell engraftment with a mean 4-fold higher levels of human CD45⁺ cells in the marrow compared to mice transplanted with day 0 BM CD34⁺CD38⁻ cells at the identical dose. Over the entire range of doses, day 0 BM CD34⁺CD38⁻ cells engrafted in 6 of 34 mice (17.6%), whereas the progeny of BM CD34⁺CD38 cells following HUBEC culture engrafted in 14 of 35 (40.0%) of NOD/SCID mice. Of note, mice transplanted with the progeny of limiting doses of HUBEC-cultured BM CD34⁺CD38⁻ cells displayed differentiation of human CD34⁺ progenitor cells (15.8%±6.1), CD 13⁺ myeloid (28.7%±8.3), and CD19⁺ B lymphoid cells (56.1%±11.3) which was comparable to that observed in mice engrafted with Day 0 BM CD34⁺CD38⁻ cells, indicating that primitive cells with multilineage differentiation potential were maintained during HUBEC culture.

For statistical analysis of SRC frequency within day 0 BM CD34⁺CD38⁻ cells and HUBEC+GM36SF cultured BM CD34⁺CD38⁻ cells, data were pooled from the limiting dilution assays according to methods described previously (Taswell, J. Immunol. 126:1614-1619 (1981), Wang et al, Blood 89:3919-3924 (1997), Ueda et al, J. Clin. Invest. 105:1013-1021 (2000)). The frequency of SRC was calculated using the maximum likelihood estimator (Wang et al, Blood 89:3919-3924 (1997)). The SRC frequency within day 0 BM CD34⁺CD38⁻ cells was 1 in 72,000 (95% Confidence Interval: 1/35,000 to 1/182,000). The SRC frequency within HUBEC-cultured BM CD34⁺ CD38⁻ cells was 1 in 20,000 (CI: 1/12,000-1/38,000). Therefore, co-culture of BM CD34⁺CD38⁻ cells with HUBEC+GM36SF supported a 3.6-fold increase in SRC compared to input. The difference in the SRC frequencies between day 0 BM CD34⁺CD38⁻ cells and their HUBEC-cultured progeny was highly significant (P=0.009).

### Non-Contact HUBEC Culture and TSF Augment BM SRC Expansion

In order to determine whether SRC expansion during HUBEC co-culture was dependent upon cell-to-cell contact, a group of NOD/SCID mice was transplanted with the progeny of 4×10⁴ BM CD34⁺CD38⁻ cells cultured with HUBEC+GM36SF in the absence of contact. Remarkably, mice transplanted with the progeny of non-contact HUBEC cultures demonstrated 8-fold higher human CD45⁺ cell repopulation at 8 weeks than mice transplanted with the progeny of contact HUBEC cultures (mean 19.5% huCD45⁺ cells versus 2.4% hu-CD45⁺ cells, P=0.01; FIG. 4). This result suggested that a significant percentage of primitive BM repopulating cells may have remained adherent to HUBEC monolayers and unevaluated following HUBEC contact cultures. These data also indicated that cell-to-cell contact was not required for HUBEC to impart SRC expansion.

Since HUBEC culture+TSF supported the largest expansion of BM CD34⁺CD38⁻ cells, a group of NOD/SCID mice was transplanted with the progeny of 4×10⁴ BM CD34⁺CD38⁻ cells following HUBEC culture+TSF. As shown in FIG. 4, mice transplanted with the progeny of HUBEC+TSF cultured cells also demonstrated high levels of human repopulation at 8 weeks (mean 18.0% hu CD45⁺ cells) which was comparable to that observed in mice transplanted with the progeny of non-contact HUBEC cultures+GM36SF and superior to that observed in mice transplanted with the progeny of HUBEC contact cultures+GM36SF. These data indicated that the substitution of TSF for GM36SF further optimized the expansion of BM SRC.

Human SRC are Enriched within the CD34⁺CD38⁻ Subset Following Ex Vivo Culture

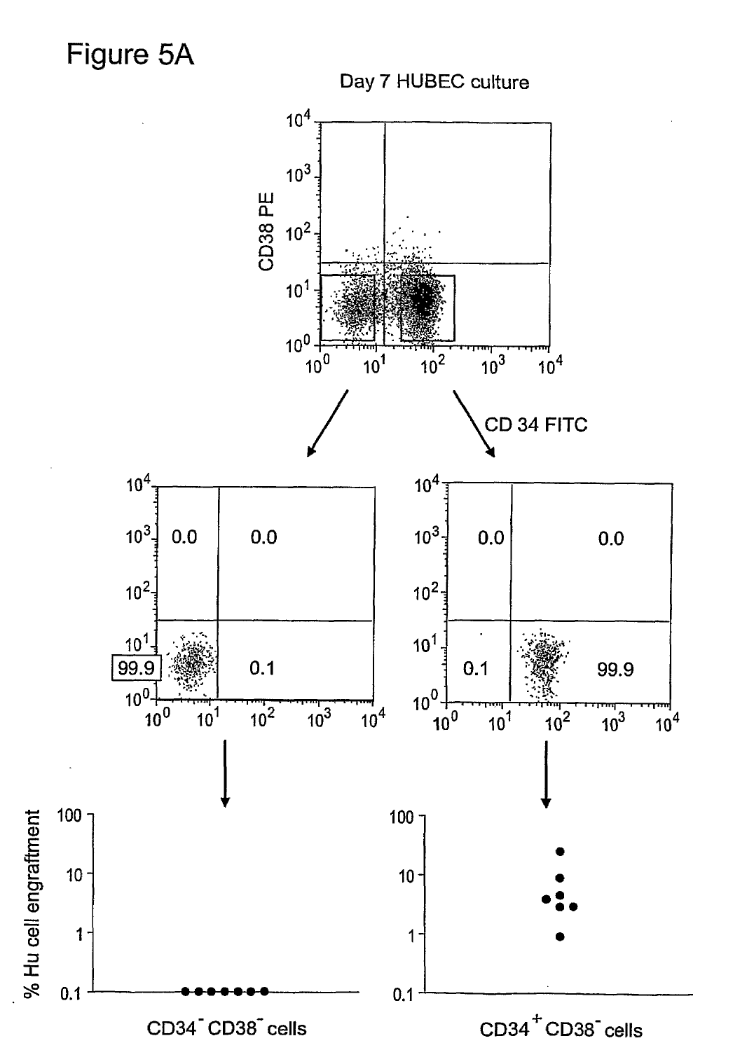

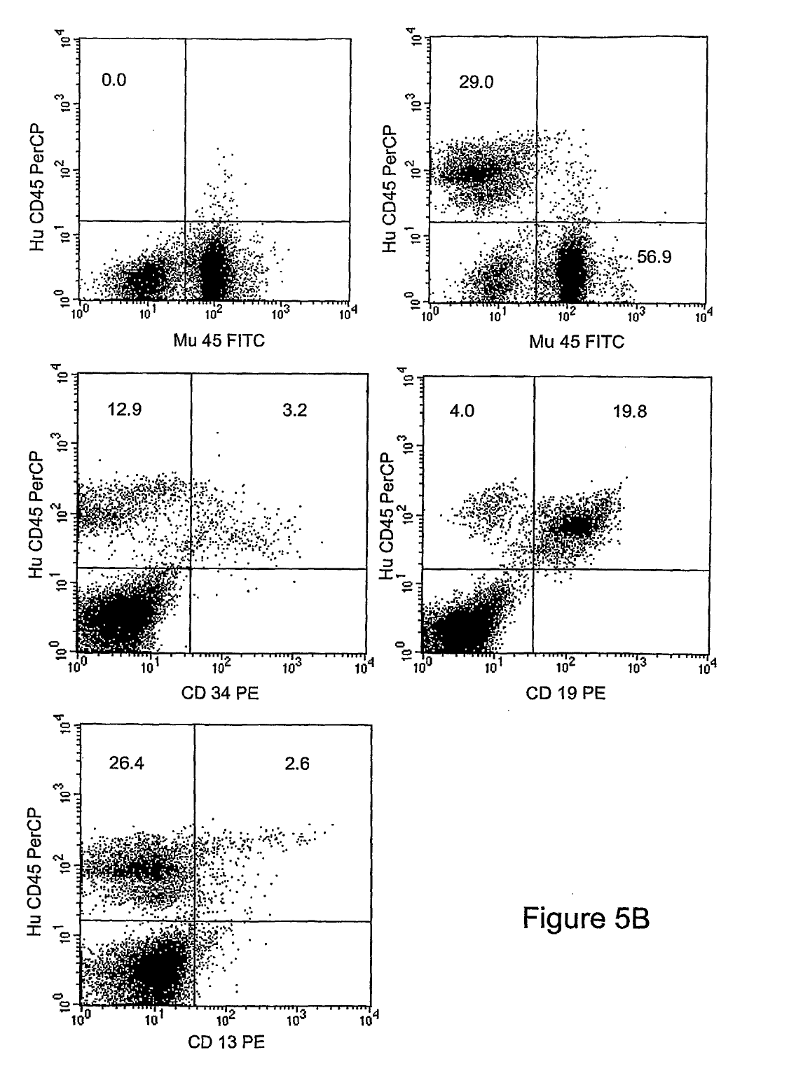

In order to determine the phenotype of primitive SRC which were amplified during HUBEC culture, we harvested the day 7 progeny of 4×10⁴ BM CD34⁺CD38⁻ cells following culture with HUBEC+GM36SF were harvested, and then, via FACS sorting, the phenotypic subsets which remained post-culture were isolated. Specifically, the CD34⁺CD38⁻ population and the CD34⁻CD38⁻ subset which encompassed >95% of day 7 HUBEC-cultured cells (FIG. 2A) were collected. NOD/SCID mice were then transplanted with the different FACS-sorted subsets to determine which population contained SRC activity. As shown in FIG. 5A, 100% of mice (n=7) transplanted with day 7 CD34⁺CD38⁻ cells showed human CD45⁺ cell repopulation at 8 weeks post-transplant, whereas 0 of 7 mice transplanted with day 7 FACS-sorted CD34⁻CD38⁻ cells showed detectable human cell repopulation. These data indicated that SRC were highly enriched within the CD34⁺CD38⁻ population compared to the CD34⁻CD38⁻ subset following HUBEC culture. Of note, human CD45⁺ cell engraftment in mice transplanted with day 7 CD34⁺CD38⁻ sorted cells was 7.0%±8.3 compared to 2.4%±2.4 in mice transplanted with the total progeny of HUBEC cultures at day 7. These data demonstrated that CD34− accessory cells did not augment the engraftment of SRC following HUBEC culture and therefore did not account for the increase in SRC frequency measured following HUBEC culture. FIG. 5B illustrates the human multilineage repopulation in a representative NOD/SCID mouse transplanted with day 7 FACS-sorted CD34⁺CD38⁻ cells compared to day 7 FACS-sorted CD34⁻CD38⁻ cells. Mice transplanted with HUBEC-cultured Day 7 CD34⁺CD38⁻ cells gave rise to multilineage repopulation in vivo, with 23.9%±3.6, 70.2%±4.6, and 28.0%±10.8 of the engrafted human CD45⁺ cells co-expressing CD34, CD19, and CD13 antigens, respectively. These data confirmed the primitive differentiative capacity of the CD34⁺CD38⁻ cell subset following HUBEC culture.

Summarizing, the application of monoclonal antibodies that recognize human CD34 and CD38 surface antigens allows convenient analysis and isolation of steady state hematopoietic cells that are enriched for stem (CD34⁺CD38⁻) and progenitor cell content (Bhatia et al, Proc. Natl. Acad. Sci. USA 94:5320-5325 (1997), Larochelle et al, Nat. Med. 2:1329-1337 (1996), Dorrell et al, Blood 95:102-110 (2000), Ishikawa et al, Leukemia 17:960-964 (2003), Hao et al, Blood 88:3306-3313 (1996)). CD34⁺ cell content correlates with clinical hematopoietic recovery following autologous and allogeneic stem cell transplantation (Siena et al, J. Clin. Oncol. 18:1360-1377 (2000), Weaver et al, Blood 86:3961-3969 (1995)) and the CD34⁺CD38⁻ phenotype identifies hematopoietic cells enriched for long-term repopulating capacity as demonstrated in the NOD/SCID experimental model (Bhatia et al, Proc. Natl. Acad. Sci. USA 94:5320-5325 (1997), Larochelle et al, Nat. Med. 2:1329-1337 (1996), Dorrell et al, Blood 95:102-110 (2000). A phenotypic indicator of HSC content would also be valuable following ex vivo culture of CD34⁺ and CD34⁺CD38⁻ subsets, since ex vivo expansion has a potential role in adult CB transplantation (Jaroscak et al, Blood 101:5061-5067 (2003)), development of tolerizing hematopoietic grafts (Gur et al, Blood 99:4174-4181 (2002), gene therapy (Piacibello et al, Blood 100:4391-4400 (2002)), and tissue generation/repair (Bailey et al, Blood 103:13-19 (2004)). However, recent studies have suggested that the CD34⁺CD38⁻ phenotype is not a reliable indicator of HSC content following ex vivo culture of CD34⁺ cells due to the downmodulation of CD38 surface antigen which occurs on committed CD34⁺CD38⁺ cells contained in culture (Dorrell et al, Blood 95:102-110 (2000)). In this study, it has been demonstrated both directly and indirectly that the most primitive assayable human hematopoietic cell, the SRC, can be monitored via expression of the CD34⁺CD38⁻ phenotype following ex vivo expansion. When HUBEC cultures were initiated with purified BM CD34⁺CD38⁻ cells, the subsequent expansion of CD34⁺CD38⁻ cells (4.4-fold) correlated well with SRC expansion (3.6-fold) over the same time period. Furthermore, flow cytometric sorting and transplantation of purified CD34⁺CD38⁻ and CD34⁻CD38⁻ subsets following HUBEC culture demonstrated that SRC were significantly enriched post-culture within the CD34⁺CD38⁻ population. Therefore, when ex vivo culture studies are initiated with HSC-enriched CD34⁺CD38⁻ cells, persistence of the CD34⁺CD38⁻ population during culture is a reliable indicator of HSC content. The correlation between CD34⁺CD38⁻ phenotype and HSC content following culture can be exploited to allow the collection and more precise analysis of HSC that have undergone expansion.

Limiting dilution analysis in this study demonstrated that contact culture of BM CD34⁺CD38⁻ cells with HUBEC supplemented with GM36SF induced a 3.6-fold increase in SRC compared to input BM CD34⁺CD38⁻ cells. Surprisingly, culture of BM CD34⁺CD38⁻ cells with HUBEC+GM36SF in the absence of cell-to-cell contact resulted in 8-fold higher huCD45⁺ cell SCID repopulation compared to mice transplanted with contact HUBEC cultured cells. Recent studies indicated that CB SRC could be maintained equally under contact and non-contact HUBEC cultures (Chute et al, Stem Cells 22:202-215 (2004)), but the augmented expansion of adult BM SRC under non-contact conditions presented here suggests a unique interaction between HUBEC-soluble factors and primitive CD34⁺CD38⁻ cells. Since 10-20% of plated hematopoietic cells become tightly adherent to HUBEC during culture, non-contact cultures may simply provide a higher yield of primtive SRC compared to contact cultures. It is also possible, as suggested by studies of stromal culture (Verfaillie et al, Leukemia 10:498-504 (1996)), that cell-to-cell contact between HSC and HUBEC may provide relative inhibition of stem cell proliferation, resulting in lower SRC amplification compared to non-contact cultures. Interestingly, in contrast to the cell contact dependent hematopoietic activities of several reported murine and human stromal cell lines (Punzel et al, Exp. Hematol. 31:339-347 (2003), Kawada et al, Exp: Hematol. 27:904-915 (1999), Kawano et al, Blood 101:532-540 (2003)), HUBEC appear to elaborate soluble factors which potently expand primitive human HSC. A subtractive gene expression analysis of HUBEC compared to non-brain EC has been undertaken to identify candidate genes and secreted gene products that account for this unique hematopoietic activity.

In addition to the benefit of non-contact HUBEC culture conditions, it was observed that the addition of TSF to HUBEC cultures augmented SRC expansion beyond what had been observed with HUBEC+GM36SF. In prior studies, TSF with and without IL-6/IL-6 receptor, has optimized the maintenance of CB SRC during short-term and extended cultures (Piacibello et al, Blood 93:3736-3749 (1999), Tanavde et al, Exp. Hematol. 30:816-823 (2002), Ueda et al, J. Clin. Invest. 105:1013-1021 (2000)). In this study, the combination of HUBEC plus TSF resulted in 7.5-fold higher SCID repopulation compared to HUBEC plus GM36SF. Of note, both HUBEC+TSF cultures and non-contact HUBEC+GM36SF cultures augmented CD34⁺CD38⁻ cell expansion compared to contact HUBEC+GM36SF cultures (13.1-fold and 6.4-fold vs. 4.4-fold, respectively) and this appeared to correlate, although not linearly, with increasing SCID-repopulating cell capacity in these groups. The additive effect of TSF on HUBEC cultures may be a direct result of thrombopoietin stimulation of HOXB4 expression in HSC (Kawano et al, Blood 101:532-540 (2003)) or may reflect the activity of these cytokines, individually and in combination, on HSC maintenance in vitro (Kirito et al, Blood 102:3172-3178 (2003), Dao et al, Blood 89:446-456 (1997), Goff and Greenberger, Blood 92:4098-4107 (1998)). Alternatively, endothelial cells express C-mpl and c-kit receptors (Broudy et al, Blood 83:2145-2152 (1994), Perlingeiro et al, Stem Cells 21:272-280 (2003), Brizzi et al, Circ. Res. 84:785-796 (1999)) and it is plausible that TPO or SCF may signal HUBEC to secrete other factors which promote HSC expansion during culture. Via limiting dilution analysis, an attempt will be made to quantify the individual and combined effects of non-contact HUBEC culture and the addition of TSF on human SRC expansion. To date, non-contact HUBEC culture conditions and TSF appear to optimize the ex vivo expansion of adult human HSC.

Previous studies have suggested that CD34⁻ and/or CD34⁺CD38⁺ accessory cells contained within transplant grafts facilitate the engraftment of limiting doses of primitive HSC (Bonnet et al, Bone Marrow Transpl. 23:203-209 (1999), Knobel et al, Exp. Hematol. 22:1227-1235 (1994)). Bonnet et al. showed that engraftment of limiting doses of CB CD34⁺ cells in NOD/SCID mice was significantly improved when CD34⁻ or CD34⁺CD38⁺ cells were co-transplanted Bonnet et al, Bone Marrow Transpl. 23:203-209 (1999)). In previous studies, it was postulated that the generation of CD34⁻ or CD34⁺CD38⁺ accessory cells during HUBEC culture may have accounted for the increased sap repopulating capacity observed in HUBEC-cultured progeny (Chute et al, Blood 100:4433-4439 (2002)). In this study, FACS-sorted day 7 CD34⁺CD38⁻ cells demonstrated equal or greater SCID repopulating capacity (mean 7.2% huCD45⁺ cell repopulation) than the total cultured day 7 progeny (mean 2.4% huCD45⁺) which contained CD34⁻ cells. Therefore, accessory cells contained within HUBEC-cultured grafts clearly do not account for the increased SRC capacity of HUBEC-cultured progeny. Rather, these data indicate that the SRC expansion observed during HUBEC culture reflects the amplification of stem/repopulating cells. The results also raise the question whether human HSC expansion might be improved via initiation of expansion cultures with purified CD34⁺CD38⁻ cells rather than heterogeneous CD34⁺ cells. Previous studies have shown that unselected human CD34⁺ populations elaborate many cytokines in culture, including TNF-alpha, IFN-alpha, and IL-1 beta, which could have deleterious effects on the ex vivo maintenance of HSC (Majka et al, Blood 97:3075-3085 (2001)). Experiments are being undertaken to answer this question by comparing the SRC expansion of human CD34⁺CD38⁻ cells cultured with and without CD34⁺CD38⁺ committed progenitors. If these studies confirm that human SRC expansion is significantly improved via exclusion of CD34⁺CD38⁺ cells from culture, these results could have implications for strategies to clinically expand HSC for transplantation. In addition, the studies have not yet addressed the potential for HUBEC culture to expand SRC contained in steady state human CD34⁻ cells (Bhatia et al, Nat. Med. 4:1038-1045 (1998), Gao et al, Exp. Hematol. 29:910-921 (2001)). Although this study suggests little, if any SRC activity within CD34⁻CD38⁻ cells derived from the input CD34⁺CD38⁻ fraction, long-term culture of input CD34⁻ cells with HUBEC may yield additional-increases in SRC content (Bhatia et al, Nat. Med. 4:1038-1045 (1998)).

The application of flow cytometry and lineage depletion to isolate steady state murine and human HSC has allowed remarkable molecular characterization of these rare cells (Terskikh et al, Blood 102:94-101 (2003), Park et al, Blood 99:488-498 (2002), Ivanova et al, Science 298:601-604 (2002), Oh et al, Blood 96:4160-4168 (2000)). Similarly, flow cytometric isolation of human HSC which have undergone self-renewal divisions has the potential to allow definitive insights into the signals involved in the HSC self-renewal process. However, the lack of determination of the surface phenotype of self-renewing HSC has impeded such progress. The model described demonstrates that primitive human repopulating cells are enriched within the CD34⁺CD38⁻ subset following culture conditions in which SRC amplification has occurred. When purified BM CD34⁺CD38⁻ cells are utilized to initiate culture, the persistence of the CD34⁺CD38⁻ population is a reliable indicator of HSC content and the expansion of this population correlates well with an increase in long term repopulating cells. It is anticipated that the results of this study will allow more definitive characterization of human HSC as they undergo expansion as well as the signals that mediate this process.

### Example 2

### RNA Isolation and Gene Expression Analysis

Primary human brain endothelial cells were placed in culture as previously described: HUBEC monolayers were established in culture as previously described. Briefly, 1×10⁵ HUBEC cells were cultured on gelatin-coated 6-well plates (Costar, Cambridge, Mass.) in complete endothelial cell culture medium (5 ml/well) containing M199 (Invitrogen), 10% FBS, 100 μg/mL L-glutamine (Invitrogen), 50 μg/mL heparin, 60 μg/mL endothelial cell growth supplement (Sigma. St. Louis, Mo.), and pcn/strp at 37° C. in 5% CO, atmosphere. For analysis of steady state HUBEC gene expression, confluent HUBECs were cultured×7 days, washed×2, trypsinized and the cells were pelleted and resuspended in TRIzol reagent [Sigma] for RNA preservation.

Human umbilical vein endothelial cells (HUVEC) were used as control cells and were cultured primarily as previously described [ATCC]. Briefly, 1×10⁵ HUVEC were plated in gelatin-coated 6 well plates in medium containing FI2K medium (ATCC) with 2 mM L-glutamine, 0.1 mg/mL heparin, 0.05 mg/mL EGGS and 10% FBS. At day 7, the confluent HUVEC were trypsinized, washed×2 and resuspended in TRIzol reagent for RNA preservation.

RNA isolation from HUBEC and HUVEC was performed as follows. Briefly, 5×10⁶ endothelial cells were pelleted and incubated with 1 mL TRIzol reagent and incubated×5 minutes. Cells were then mixed with 0.2 mL of chloroform×3 minutes at room temp., then centrifuged at 11,500 RPM×15 minutes at 4° C. The upper aqueous phase of the sample was then collected into RNAse free eppendorf tubes and then mixed with 0.5 mL of isopropanol×10 minutes. Samples were then centrifuged at 11,500 rpm×15 minutes at 4° C. The supernatant was then aspirated and the pellet was resuspended in 75% ethanol in DEPC-H20 by vortexing. Samples were then air-dried and RNA quantity was measured via spectrophotometry.

After RNA isolation, samples were run through RNeasy column to eliminate potential DNA and protein contamination as previously described. The samples were then precipitated with ethanol as previously described. Following ethanol precipitation, samples were analyzed via spectrophotometry and TBE ethidium bromide gel electrophoresis to verify the presence of highly pure RNA. RNA samples purified in this manner were then hybridized to Affymetrix human 133A and 133B microarrays containing >30,000 representative human gene sequences, as previously described. In order to verify consistency of gene expression within the endothelial cell samples, multiple biological replicates were subjected to microarray hybridization in an identical manner (n=5 HUBEC RNA samples, n=4 HUVEC RNA samples). Unified gene lists for each endothelial cell group representing only those genes consistently up or down-regulated were then generated.

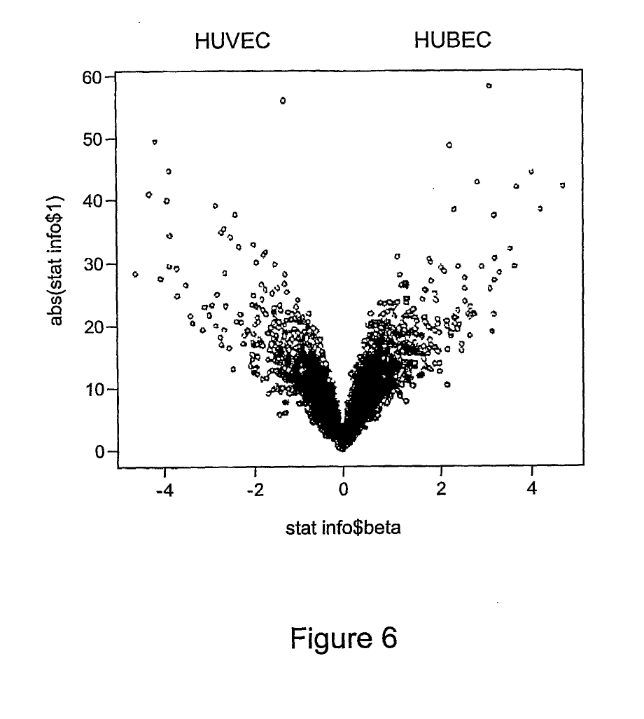

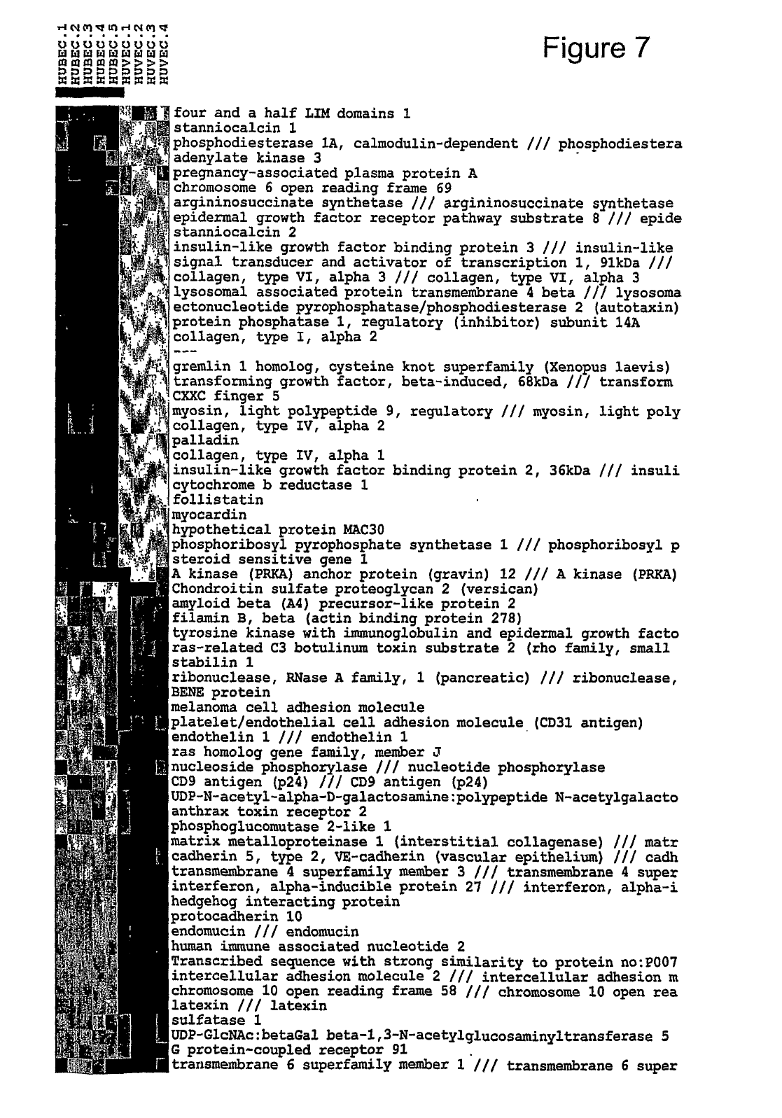

Collection of probe list data and analysis followed the MGED/MIAMI guidelines. All genes on U133A and B chips were re-annotated into 26,570 non-redundant unigene identifiers. An unsupervised cluster analysis using all these genes suggests a difference between the transcriptional programs of HUBEC and HUVEC. To statistically identify these differentially expressed genes, a total of 4,477,203 probes in 18 hybridizations were fitted by linear models. The top 65 genes were selected according to their t-scores of differential expression. (see Table 3 (see also FIGS. 6 and 7)) Literature analysis suggests these 65 genes are interconnected to achieve coordinated biological function. Gene Ontology analysis suggests hormone activity, extracellular space, cell-cell signaling are over-represented in this gene list. (See Table 4) Novel secreted proteins that are overexpressed in HUBEC are shown in Table 5.

TABLE 3
Unique genes that are overexpressed in HUBEC as
compared to HUVEC.
All genes on the Affymetrix U133A and B chips were combined into a non-redundant set
according to unigene identifiers. Each gene was modeled by a linear statistical model at
the probe-level. The top 65 genes were selected according to their t-scores from the
linear model. All of these 65 genes have a p-value smaller than 0.01.

| Unigene | Fold | | | |
|---|---|---|---|---|
| ID | change | t-score | Symbol | Gene Name |
| Hs.433326 | 22.7 | −28.4697 | IGFBP2 | insulin-like growth factor binding protein 2, 36 kDa |
| Hs.232115 | 18.4 | −41.001 | COL1A2 | collagen, type I, alpha 2 |
| Hs.421496 | 16.8 | −49.4707 | TGFBI | transforming growth factor, beta-induced, 68 kDa |
| AF130082 | 15.9 | −27.6196 | | Unknown |
| Hs.23719 | 14.3 | −40.1201 | ENPP2 | ectonucleotide pyrophosphatase/phosphodiesterase 2 (autota |
| Hs.348037 | 14 | −29.6537 | PPP1R14A | protein phosphatase 1, regulatory (inhibitor) subunit 14A |
| Hs.40098 | 13.8 | −44.7151 | GREM1 | gremlin 1 homolog, cysteine knot superfamily (Xenopus laevis |
| Hs.160786 | 13.7 | −34.4033 | ASS | argininosuccinate synthetase |
| Hs.433814 | 12.6 | −29.1636 | MYL9 | myosin, light polypeptide 9, regulatory |
| Hs.233240 | 11 | −26.4445 | COL6A3 | collagen, type VI, alpha 3 |
| Hs.2132 | 6.9 | −25.1668 | EPS8 | epidermal growth factor receptor pathway substrate 8 |
| Hs.450230 | 6.9 | −39.3367 | IGFBP3 | insulin-like growth factor binding protein 3 |
| Hs.56 | 6.3 | −34.9096 | PRPS1 | phosphoribosyl pyrophosphate synthetase 1 |
| Hs.437173 | 6.1 | −35.7357 | COL4A1 | collagen, type IV, alpha 1 |
| Hs.42128 | 6.1 | −28.6323 | MYOCD | myocardin |
| Hs.407912 | 5.5 | −34.2239 | COL4A2 | collagen, type IV, alpha 2 |
| Hs.356509 | 5 | −37.9042 | CXXC5 | CXXC finger 5 |
| Hs.155223 | 4.8 | −32.703 | STC2 | stanniocalcin 2 |
| Hs.356289 | 3.9 | −33.1474 | URB | steroid sensitive gene 1 |
| Hs.9914 | 3.7 | −30.1854 | FST | follistatin |
| Hs.188757 | 3.4 | −26.7281 | C6orf69 | chromosome 6 open reading frame 69 |
| Hs.194431 | 3.2 | −31.4286 | KIAA0992 | palladin |
| Hs.25590 | 3.2 | −25.9193 | STC1 | stanniocalcin 1 |
| Hs.296398 | 3.2 | −31.9694 | LAPTM4B | lysosomal associated protein transmembrane 4 beta |
| Hs.10862 | 2.9 | −25.3457 | AK3 | adenylate kinase 3 |
| Hs.440769 | 2.7 | −29.8539 | PAPPA | pregnancy-associated plasma protein A |
| Hs.31297 | 2.6 | −26.2109 | CYBRD1 | cytochrome b reductase 1 |
| Hs.199695 | 2.4 | −26.7861 | MAC30 | hypothetical protein MAC30 |
| Hs.21486 | 2.3 | −56.1385 | STAT1 | signal transducer and activator of transcription 1, 91 kDa |
| Hs.421383 | 2.3 | −25.5939 | FHL1 | four and a half LIM domains 1 |
| Hs.416061 | 2.3 | −28.4021 | PDE1A | phosphodiesterase 1A, calmodulin-dependent |
| Hs.197081 | 2.3 | −25.4848 | AKAP12 | A kinase (PRKA) anchor protein (gravin) 12 |
| Hs.279518 | −2.3 | 31.28838 | APLP2 | amyloid beta (A4) precursor-like protein 2 |
| Hs.434488 | −2.4 | 28.36924 | CSPG2 | chondroitin sulfate proteoglycan 2 (versican) |
| Hs.5897 | −2.4 | 26.73473 | ANTXR2 | anthrax toxin receptor 2 |
| Hs.277324 | −2.6 | 27.06172 | GALNT1 | UDP-N-acetyl-alpha-D-galactosamine:polypeptide, N-acetylgal [symbol] |
| | | | | T1) |
| Hs.26612 | −2.6 | 25.60272 | PGM2L1 | phosphoglucomutase 2-like 1 |
| Hs.257222 | −3.3 | 25.95915 | B3GNT5 | UDP-GlcNAc:betaGal beta-1,3-N-acetylglucosaminyltransfera [symbol] |
| Hs.75514 | −3.4 | 25.9131 | NP | nucleoside phosphorylase |
| Hs.409602 | −3.6 | 30.89745 | SULF1 | sulfatase 1 |
| Hs.105468 | −3.6 | 27.41569 | hIAN2 | human immune associated nucleotide 2 |
| Hs.78146 | −3.7 | 30.43546 | PECAM1 | platelet/endothelial cell adhesion molecule (CD31 antigen) |
| Hs.151155 | −3.9 | 25.35221 | TM6SF1 | transmembrane 6 superfamily member 1 |
| Hs.301989 | −4.2 | 29.48633 | STAB1 | stabilin 1 |
| Hs.511899 | −4.4 | 28.97667 | EDN1 | endothelin 1 |
| Hs.243010 | −4.9 | 49.26564 | RHOJ | ras homolog gene family, member J |
| Hs.81008 | −5.1 | 38.824 | FLNB | filamin B, beta (actin binding protein 278) |
| Hs.511397 | −5.4 | 29.71115 | MCAM | melanoma cell adhesion molecule |
| Hs.387579 | −5.9 | 26.16374 | CD9 | CD9 antigen (p24) |
| Hs.301175 | −5.9 | 27.84813 | RAC2 | ras-related C3 botulinum toxin substrate 2 (rho family, small [symbol] |
| Hs.463538 | −7.6 | 43.29464 | | Transcribed sequence with strong similarity to protein sp.P00 [symbol] |
| Hs.78824 | −7.9 | 29.80102 | TIE | tyrosine kinase with immunoglobulin and epidermal growth fa [symbol] |
| Hs.76206 | −9 | 26.11165 | CDH5 | cadherin 5, type 2, VE-cadherin (vascular epithelium) |
| Hs.41135 | −9.3 | 58.46405 | EMCN | endomucin |
| Hs.84072 | −9.5 | 27.58264 | TM4SF3 | transmembrane 4 superfamily member 3 |
| Hs.185055 | −9.7 | 31.10791 | BENE | BENE protein |
| Hs.413504 | −9.7 | 37.87727 | HHIP | hedgehog interacting protein |
| Hs.124491 | −9.8 | 38.03536 | LXN | latexin |
| Hs.279575 | −10.5 | 28.68902 | GPR91 | G protein-coupled receptor 91 |
| Hs.146858 | −12.2 | 32.5373 | PCDH10 | protocadherin 10 |
| Hs.78224 | −13.1 | 29.80438 | RNASE1 | ribonuclease, RNase A family, 1 (pancreatic) |
| Hs.495628 | −13.6 | 42.49424 | C10orf58 | chromosome 10 open reading frame 58 |
| Hs.433303 | −17.2 | 44.82385 | ICAM2 | intercellular adhesion molecule 2 |
| Hs.278613 | −19.4 | 38.9192 | IFI27 | interferon, alpha-inducible protein 27 |
| Hs.83169 | −27.4 | 42.66206 | MMP1 | matrix metalloproteinase 1 (interstitial collagenase) |

[symbol]

indicates data missing or illegible when filed

TABLE 4
Organization of 65 unique HUBEC genes based upon gene
ontology.

| | # of | | |
|---|---|---|---|
| | gene in | | |
| | the 65- | | |
| | gene | Fold of | EASE |
| Gene Ontology Category | list | Enrichment | P-value |
| cellular component | | | |
| extracellular | 17 | 3.86 | 0.000 |
| extracellular matrix | 7 | 5.60 | 0.001 |
| extracellular space | 8 | 5.10 | 0.001 |
| collagen | 4 | 26.42 | 0.000 |
| integral to membrane | 19 | 1.41 | 0.077 |
| biological process | | | |
| cell communication | 25 | 1.96 | 0.000 |
| cell-cell signaling | 5 | 2.08 | 0.206 |
| cell-cell adhesion | 5 | 4.93 | 0.017 |
| cell adhesion | 11 | 4.31 | 0.000 |
| organogenesis | 8 | 2.19 | 0.062 |
| development | 14 | 2.05 | 0.011 |
| regulation of cell growth | 3 | 8.29 | 0.049 |
| cell growth | 4 | 8.04 | 0.013 |
| molecular function | | | |
| hormone activity | 3 | 6.52 | 0.075 |
| binding | 34 | 1.11 | 0.233 |
| extracellular matrix | 4 | 10.10 | 0.007 |
| structural constituent | | | |

TABLE 5
Novel secreted proteins that are overexpressed within
HUBEC

| | | Fold |
|---|---|---|
| Gene ID | Gene Name | Increase |
| Hs.433326 | insulin-like growth factor binding protein 2 | 22.7 |
| Hs.421496 | transforming growth factor, beta-induced | 16.8 |
| Hs.40098 | cystein knot superfamily 1 | 13.8 |
| Hs.65436 | lysyl oxidase-like 1 | 12.6 |
| Hs.458354 | thrombospondin 2 | 10.3 |
| Hs.152213 | wingless-type MMTV integration site family, 5A | 9.6 |
| Hs.147697 | nidogen 2 | 8.6 |
| Hs.334702 | lysyl oxidase-like 3 | 7.1 |
| Hs.450230 | insulin-like growth factor binding protein 3 | 6.9 |
| Hs.303649 | chemokine ligand 2 | 6.0 |
| Hs.155223 | stanniocalcin | 4.8 |
| Hs.65424 | tetranectin | 4.8 |
| Hs.441047 | adrenomedullin | 4.3 |
| Hs.156316 | decorin | 4.0 |
| Hs.79339 | lectin, galactoside-binding, soluble, 3 binding prot. | 4.0 |
| Hs.285671 | bone morphogenetic protein 6 | 3.9 |
| Hs.9914 | follistatin | 3.7 |
| Hs.198862 | fibulin 2 | 3.7 |
| Hs.46721 | upregulated in colorectal cancer gene 1 | 3.4 |
| Hs.274313 | insulin-like growth factor binding protein 6 | 2.8 |
| Hs.80420 | chemokine (C—X3—C motif) ligand 1 | 2.7 |
| Hs.440769 | pregnancy-associated plasma protein A | 2.7 |
| Hs.1516 | insulin-like growth factor binding protein 4 | 2.4 |
| Hs.406475 | lumican | 2.3 |
| Hs.7306 | secreted frizzled related protein 1 | 1.9 |
| Hs.409202 | jagged 1 | 1.9 |
| Hs.194680 | WNT1 inducible signaling pathway protein 1 | 1.8 |
| Hs.433319 | cardiotrophin | 1.5 |

The transcripts identified represent selected genes identified to have cell-cell signaling, hormone, cell adhesion, or extracellular function.

### Example 3

Hematopoietic stem cells (HSCs) possess the unique capacity to undergo self-renewal in vivo throughout the life of an individual while also providing the complete repertoire of mature hematopoietic and immune cells [1-3]. Currently, transplantation of human HSCs from adult bone marrow (BM), mobilized peripheral blood, and umbilical cord blood (CB) is applied in the curative treatment of both malignant and nonmalignant diseases [4-6]. More recently, the potential contribution of transplanted HSCs toward immune tolerance induction [7], vascular repair [8], and in vivo tissue regeneration [9] has been suggested. Since HSCs comprise <0.1% of the CD34⁺ population within the bone marrow of adults [10], numerous studies have focused on the development of methods to expand HSC numbers in vitro with a goal of generating larger numbers of transplantable repopulating cells [11-14]. Concordantly, strategies have been applied to identify novel growth factors that stimulate HSC self-renewal in vivo [15-18]. Despite these efforts, few hematopoietic growth factors have achieved clinical application [19-21].

One strategy to identify HSC growth factors involves examination of candidate niches wherein HSCs are known to reside physiologically [22-24]. Two recent studies have demonstrated that HSCs reside in contact with osteoblasts in the BM niche [23, 24] and these cells provide signaling through Notch ligand and cadherin interactions to maintain quiescent HSCs in vivo. A vascular niche within the marrow has also been postulated, comprised of sinusoidal endothelial cells, in which HSC proliferation and differentiation are thought to occur [22]. The role of endothelial cells (ECs) as regulators of hematopoiesis is supported by evidence from embryogenesis, in which development of blood islands is critically dependent upon the presence of flk-1-positive vascular precursor cells [25, 26]. Gene marking studies have also suggested a common precursor cell, the hemangioblast, which appears to give rise to both HSCs and endothelial precursor cells [27]. Yolk sac ECs support hematopoietic progenitor cell growth ex vivo [28], and adult BM ECs support the in vitro proliferation of erythroid, myeloid, and megakaryocytic progenitors [29, 30]. Anatomically, human HSCs embed within the intimal layer of the aorta at day 35 of embryogenesis [3] and reside in association with ECs in the fetal liver [26] and, ultimately, in the adult BM [22, 32]. Therefore, ECs are a logical source of growth factors that regulate HSC growth and differentiation.

Several studies have examined the capacity for stromal cell lines to support the ex vivo maintenance of HSCs [14, 33, 34]. Whereas studies of primary human BM stroma have been disappointing [33], a murine fetal liver stromal cell line, AFT024, has been shown to support the maintenance of human CB severe combined immunodeficient-repopulating cell (SRCs) in vitro [34, 35]. Conversely, our laboratory has shown that coculture with brain-derived porcine microvascular endothelial cells supports the expansion of human BM CD34⁺ and CD34⁺CD38 cells during short-term culture [36]. We subsequently showed that coculture with porcine brain ECs augmented the genetic modification of human HSCs [37] and, remarkably, induced the functional repair and expansion of lethally irradiated murine HSCs [38]. In collaboration with Brandt et al. [39], we also showed that the progeny of BM cells cultured with porcine brain ECs were capable of providing long term repopulation in lethally irradiated baboons. Our recent studies indicate that this HSC-supportive activity is conserved within primary human brain ECs (HUBECs) as well, whereas nonbrain ECs fail to maintain human CD34⁺CD38⁻ cells in culture [13]. Coculture with HUBECs supports a four fold expansion of both human BM SRCs [13] and CB SRCs [40] in 7-day cultures, and in contrast to comparative stromal cell lines [34], cell-to-cell contact does not appear to be required for HUBECs to stimulate the expansion of human HSCs [40, 41]. These data suggest that soluble factors elaborated by HUBECs account for the unique hematopoietic activity that we have observed.

In this study, we have developed a molecular profile of HUBECs via comparative gene expression analysis to identify the candidate novel molecules responsible for this HSC-supportive activity. Secreted factors, extracellular proteins, and cell-cell signaling proteins are highly overrepresented within the HUBEC transcriptome. Moreover, initial functional analyses indicate that a vasoactive peptide, adrenomedullin, synergizes with other cytokines to induce human progenitor cell proliferation and expansion.

### Materials and Methods

Noncontact Cultures of Human CB CD34⁺ Cells with Primary HUBECs

Primary human cord blood CD34⁺ cells were procured from Cambrex (Cambrex, Walkersville, Md., http://www.cambrex.com). Briefly, 1×10⁵ CD34⁺ cells were placed in six-well culture plates with Iscove's modified Dulbecco's medium (IMDM) (Gibco-BRL, Gaithersburg, Md., http://www.gibcobrl.com) with 10% fetal calf serum and 1% penicillin/streptomycin (pcn/strp) (Gibco-BRL) supplemented with 20 ng/ml thrombopoietin, 120 ng/ml stem cell factor, and 50 ng/ml Flt-3 ligand (TSF) (R&D Systems Inc., Minneapolis, http://www.rndsystems.com) or in noncontact cultures with primary human brain endothelial cells (RMLS-01) supplemented with TSF for 14 days. HUBECs and primary CD34⁺ cells were separated by 0.4-μm transwell inserts (Gibco-BRL). At day 14, nonadherent cells were collected from each culture condition and washed, and cell counts were obtained. Immunophenotypic analysis using fluorescent monoclonal antibodies CD34 and CD38 and appropriate isotype controls (Becton, Dickinson and Company, Franklin Lakes, N.J., http://www.bd.com) was performed at day 0 and day 14 to compare the hematopoietic content at each time point.

Transplantation of Human Hematopoietic Cells into NOD/SCID Mice

Six- to 8-week-old nonobese diabetic severe combined immunodeficient (NOD/SCID) mice (Jackson Laboratory, Bar Harbor, Me., http://www.jax.org) were used for all experiments [42]. All animal studies were performed under protocols approved by the Duke University Institutional Animal Care and Use Committee. Briefly, mice were irradiated with 300 cGy from a Cs¹³⁷ source. Four hours postirradiation, mice were transplanted via tail vein injection with either 2×10⁴ day 0 CB CD34⁺ cells or their progeny following 14-day culture with either TSF alone or HUBEC transwell cultures supplemented with TSF. Eight weeks post-transplantation, all mice were sacrificed, bilateral femurs were harvested, and BM cells were collected. Immunophenotypic analysis of human cell engraftment and lineage repopulation within the murine marrow was performed using antibodies against human CD45PerCP, anti-murine CD45 fluorescein isothiocyanate (FITC), anti-huCD34 phycoerythrin (PE), anti-huCD38FITC, anti-huCD33FITC, anti-huCD13PE, anti-huCD19PE, anti-huCD3FITC, anti-huCD56FITC, and anti-huCD71PE, along with isotype controls (Becton, Dickinson and Company). Estimation of SRC frequency in each cell source was calculated using the maximum likelihood estimator as described previously by Taswell [43] for the single-hit Poisson model [43, 44].

Isolation of RNA from HUBECs and Human Umbilical Vein Endothelial Cells and Gene Expression Analysis

Primary human brain endothelial cells were placed in culture as previously described [13]. Briefly, 1×10⁵ HUBECs were cultured on gelatin-coated six-well plates (Corning Incorporated Life Sciences, Acton, Mass., http://www.corning.com) in complete endothelial cell culture medium (5 ml per well) containing M199 (Invitrogen, Carlsbad, Calif., http://www.invitrogen.com), 10% fetal bovine serum (FBS), 100 μg/ml glutamine (Invitrogen), 50 μg/ml heparin, 60 μg/ml endothelial cell growth supplement (Sigma-Aldrich, St. Louis, http://www.sigmaaldrich.com), and 1% pcn/strp at 37° C. in 5% CO, atmosphere. For analysis of HUBEC gene expression, confluent HUBECs were cultured for 72 hours, washed twice, and trypsinized, and the cells were pelleted and resuspended in TRIzol reagent (Sigma-Aldrich) for RNA preservation.

Human umbilical vein endothelial cells (HUVECs) (ATCC, Manassas, Va., http://www.atcc.org) were used as control cells and were cultured primarily as previously described [45]. Briefly, 1×10⁵ HUVECs were plated in gelatin-coated six-well plates in medium containing FI2K medium (ATCC) with 2 mM tL-glutamine, 0.1 mg/ml heparin, 0.05 mg/ml endothelial cell growth supplement, and 10% FBS. After 72 hours, the confluent HUVECs were trypsinized, washed twice, and resuspended in Mimi reagent for RNA preservation.

RNA isolation from HUBECs and HUVECs was performed as follows. Briefly, 5×10⁶ endothelial cells were pelleted and incubated with 1 ml of TRIzol reagent and incubated for 5 minutes. Cells were then mixed with 0.2 ml of chloroform for 3 minutes at room temperature and then centrifuged at 11,500 rpm for 15 minutes at 4° C. The upper aqueous phase of the sample was then collected into RNase-free Eppendorf tubes and mixed with 0.5 ml of isopropanol for 10 minutes. Samples were then centrifuged at 11,500 rpm for 15 minutes at 4° C. The supernatant was then aspirated, and the pellet was resuspended in 75% ethanol in DEPC-H20 by vortexing. Samples were then air-dried, and RNA quantity was measured via spectrophotometry.

After RNA isolation, samples were run through an RNeasy column to eliminate potential DNA and protein contamination as previously described [46]. The samples were then precipitated with ethanol. Following ethanol precipitation, samples were analyzed via spectrophotometry and TBE ethidium bromide gel electrophoresis to verify the presence of highly pure RNA. Total RNA was used to develop the targets for Affymetrix microarray analysis and probes were prepared according to the manufacturer's instructions. Briefly, biotin-labeled cRNA was produced by in vitro transcription, fragmented, and hybridized to the Human 133A and 133B arrays (Affymetrix, Santa Clara, Calif., http://www.affymetrix.com) containing >47,000 representative human gene sequences, as previously described [47]. Arrays were hybridized at 45° C. for 16 hours and then washed and stained using the GeneChip Fluidics and scanned on the Affymetrix scanner. The hybridization signals from each array were normalized against the signals from human maintenance genes, which show consistent levels of expression across a variety of tissues prior to comparisons with other array results [48, 49]. To verify the consistency of gene expression within the endothelial cell samples, multiple biological replicates were subjected to microarray hybridization in an identical manner. Unified gene lists for each endothelial cell group, representing only those genes consistently up- or downregulated, were then generated. Collection of probe list data and analysis followed the Microarray Gene Expression Database Group/Minimum Information About a Microarray Experiment (MGED/MIAMI) guidelines [50]. All genes on U133A and B chips were reannotated into 26,570 nonredundant Unigene identifiers. An unsupervised cluster analysis using all of these genes suggested a difference between the transcriptional programs of HUBECs and HUVECs. To statistically identify these differentially expressed genes, a total of 4,477,203 probes in 18 hybridizations were fitted using gene-by-gene analysis of variance (ANOVA) linear models. All calculations were conducted using the R/Bio-conductor package [51]. The top 65 candidate genes were analyzed by gene ontology using the EASE algorithm [52].

### Quantitative Real-Time RT-PCR Analysis of HUBEC Gene Expression

Total RNA was isolated from 1×10⁶ HUBECs or HUVECs (ATCC) using the RNeasy Mini kit (Qiagen, Valencia, Calif., http://www1.qiagen.com), according to the manufacturer's protocol. Total RNA was quantified using a SmartSpec 3000 spectrophotometer (Bio-Rad, Hercules, Calif., http://www.bio-rad.com), and 2 μg per sample was reverse transcribed using the High Capacity cDNA Archive kit (Applied BioSystems, Foster City, Calif., http://www.appliedbiosystems.com), using the recommended reaction conditions. Fifty-nanogram equivalents of cDNA were then used for quantitative real-time PCR using TaqMan Gene Expression Assays (Applied Biosystems) for decorin, insulin-like growth factor binding protein 2 (IGFBP-2), myocardin, adrenomedullin, and glyceraldehyde-3-phosphate dehydrogenase (GAPDH), with an ABI Prism 7700 Sequence Detection System (Applied Biosystems). Relative gene expression between HUBECs and HUVECs was calculated using the ΔΔCt method, using GAPDH expression as a normalization reference.

### CB Processing, Lineage Depletion, and FACS Sorting

Umbilical cord blood units were obtained from the Duke University Stem Cell Laboratory within 48 hours of collection. Volume reduction was accomplished by 10-minute incubation at room-temperature with 1% Hetastarch (Abbott Laboratories, North Chicago, Ill.), followed by centrifugation at 700 rpm for 10 minutes without brake, to facilitate component separation. The buffy coat was collected and washed twice with Dulbecco's phosphate-buffered saline (DPBS) (Invitrogen) containing 10% heat-inactivated FBS (HyClone, Logan, Utah, http://www.hyclone.com), 100 U/ml penicillin, and 100 μg/ml streptomycin (1% pcn/strp; Invitrogen). Cell pellets were thoroughly resuspended in DPB+10% FBS+1% pcn/strp and overlaid onto Lymphoprep (Axis-Shield, Olso, Norway) and centrifuged at 1,500 rpm for 30 minutes without brake to isolate the mononuclear cell (MNC) fraction. MNC monolayers were collected and washed twice before proceeding to lineage marker depletion.

Lineage depletion was conducted using the Human Progenitor Enrichment Cocktail (Stem Cell Technologies, Vancouver, BC, Canada), which contains monoclonal antibodies to human CD2, CD3, CD14, CD16, CD9, CD56, CD66b, and Glycophorin A, according to the manufacturer's suggested protocol. Briefly, CB MNCs were resuspended at 5-8×10⁷ cells per ml in DPBS+10% FBS+1% pcn/strp, and incubated with 100 μl/ml antibody cocktail for 30 minutes on ice, followed by incubation with 60 μl/ml magnetic colloid for 30 minutes on ice. Cells were then magnetically depleted on a pump-fed negative selection column (Stem Cell Technologies), using the manufacturer's recommended procedure. Lin⁻ cells were washed twice, quantified by manual hemacytometer count using trypan blue exclusion dye (Invitrogen), and cryopreserved in 90% FBS+10% dimethylsulfoxide (Sigma-Aldrich) or used for further experimentation.

Lin⁻ CB cells were thawed, washed once in IMDM (Invitrogen) containing 10% FBS and 1% pcn/strp, counted, and resuspended at 5×10⁶ to 1×10⁷ cells per ml. Immunofluorescent staining was conducted using anti-human CD34-FITC and anti-human CD38-PE monoclonal antibodies (Becton, Dickinson and Company) for 30 minutes on ice. Stained cells were washed twice and resuspended at 1×10⁷ cells per ml in IMDM+10% FBS+1% pcn/strp. Sterile cell sorting was conducted using a FACSvantage flow cytometer (Becton, Dickinson and Company) to isolate CD34⁺CD38⁻ and CD34⁺CD38⁺ subsets: For proliferation experiments, cells were automatically sorted into 60-well Terasaki plates (Nunclon, Rochester, N.Y.), containing 5 id per well of the appropriate growth factor media. The CD34⁺CD38⁻ sort gate was set to collect only those CD34⁺events falling in the lowest 5% of PE fluorescence within the total CD34⁺ population, as determined by staining with isotype-matched mouse IgG, controls (BD Biosciences), to ensure acquisition of highly purified CD34⁺CD38⁻ cells.

To screen for hematopoietic activity of HUBEC-secreted growth factors, we placed human CB CD34⁺ cells in culture with 50 ng/ml thrombopoietin, 100 ng/ml stem cell factor, and 50 ng/ml Flt-3 ligand (TSF) for 7 days with and without supplementation with the following recombinant proteins that we found to be differentially overexpressed by HUBECs: IGFBP2 (R&D Systems), IGFBP3 (R&D Systems), follistatin (R&D Systems), and adrenomedullin (R&D Systems).

### Results

### Soluble Factors Elaborated by HUBECs Support the Expansion of Human HSC

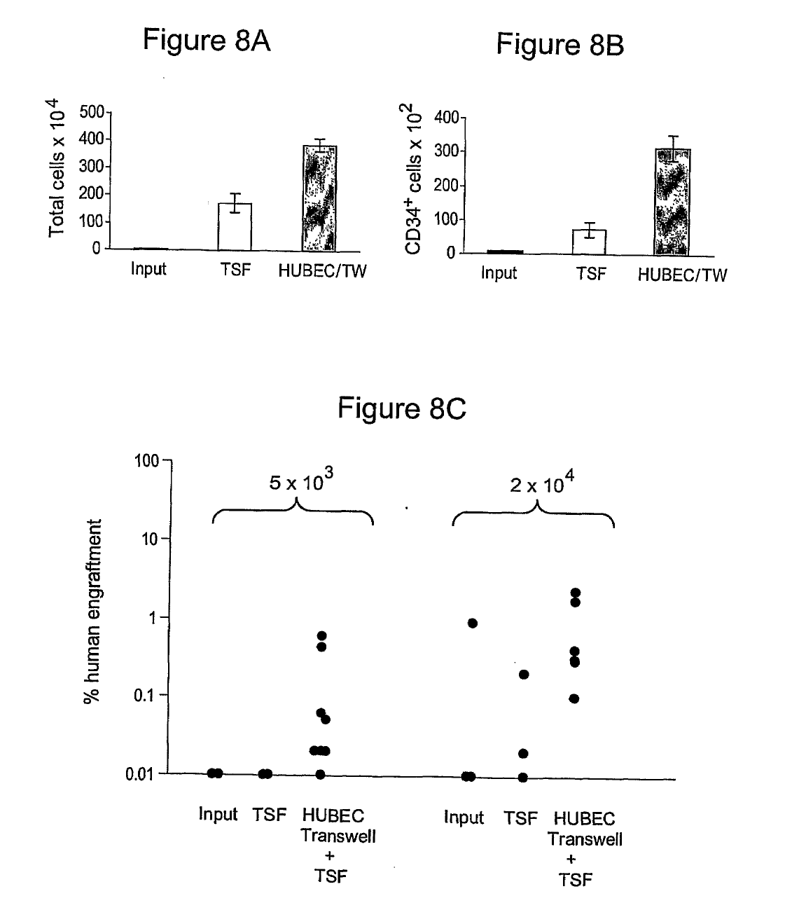

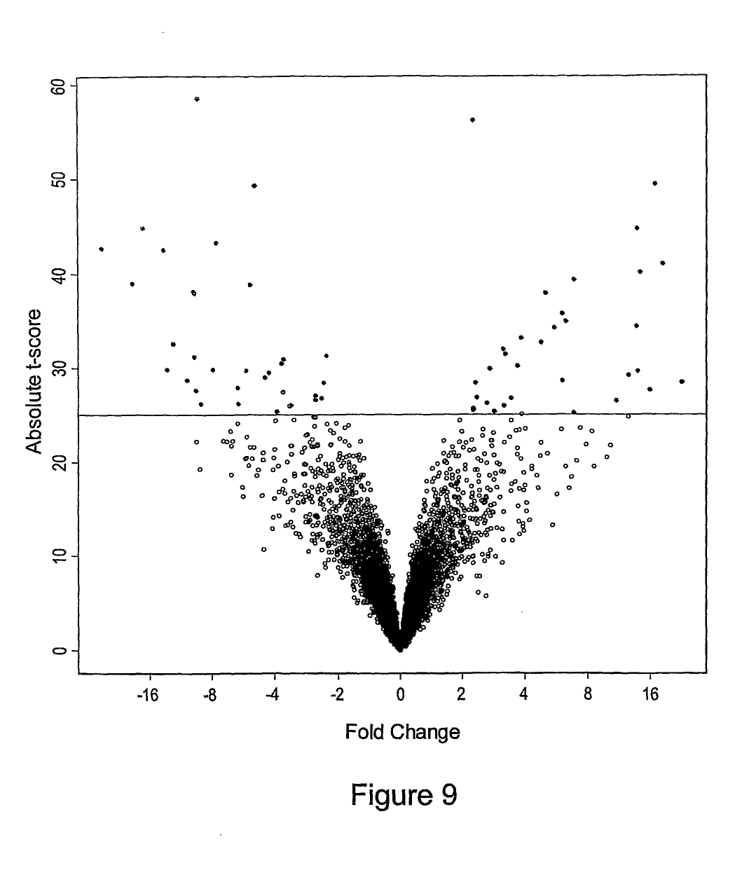

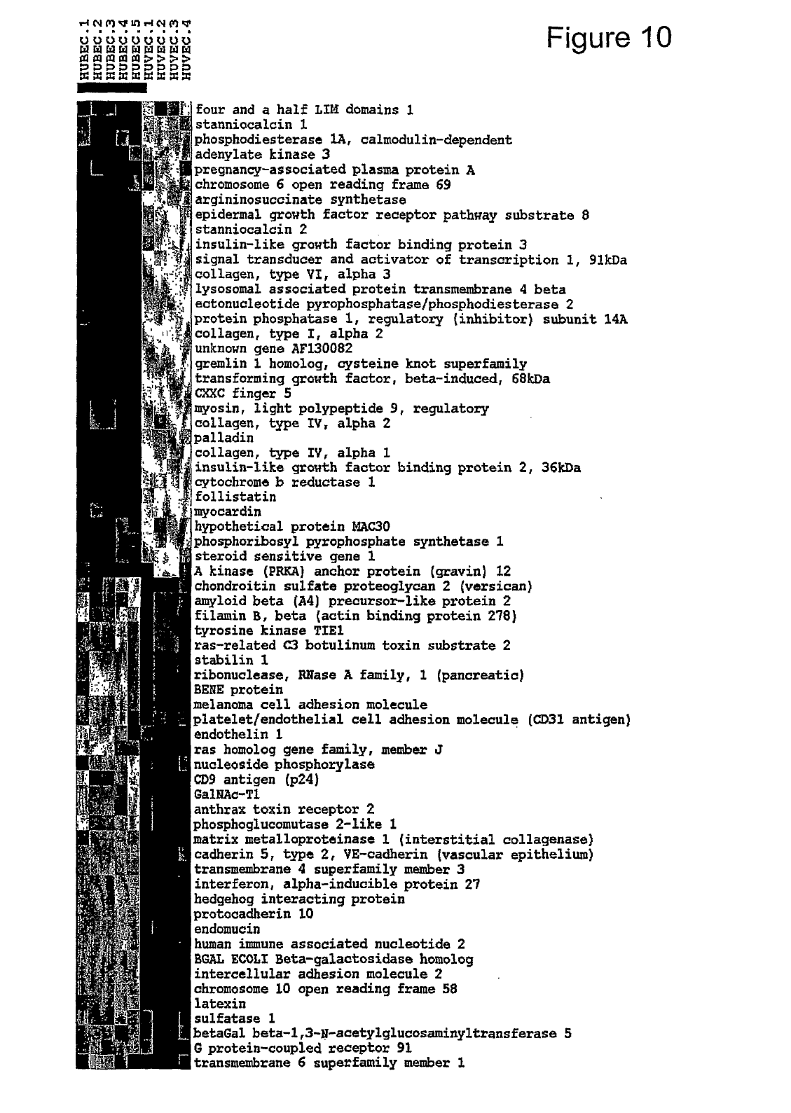

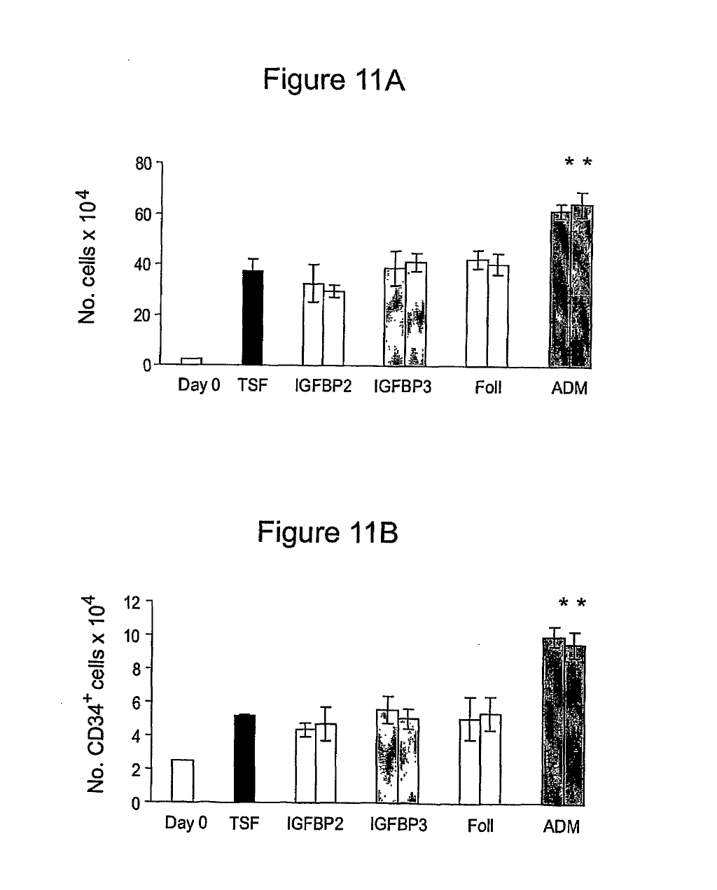

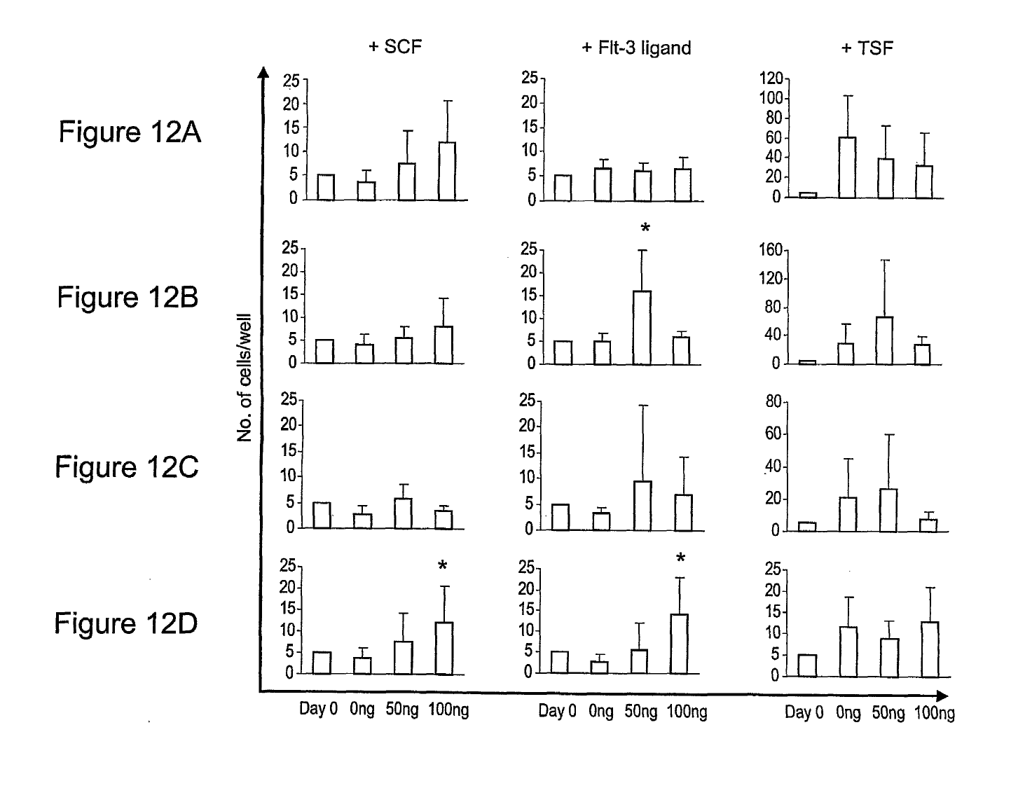

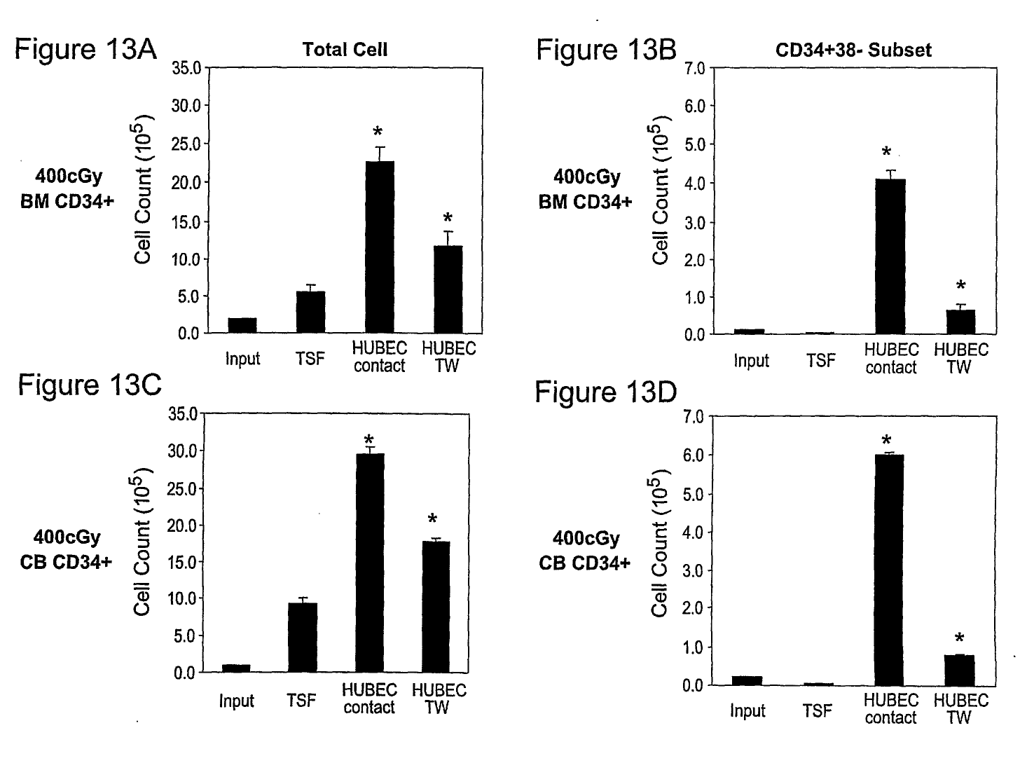

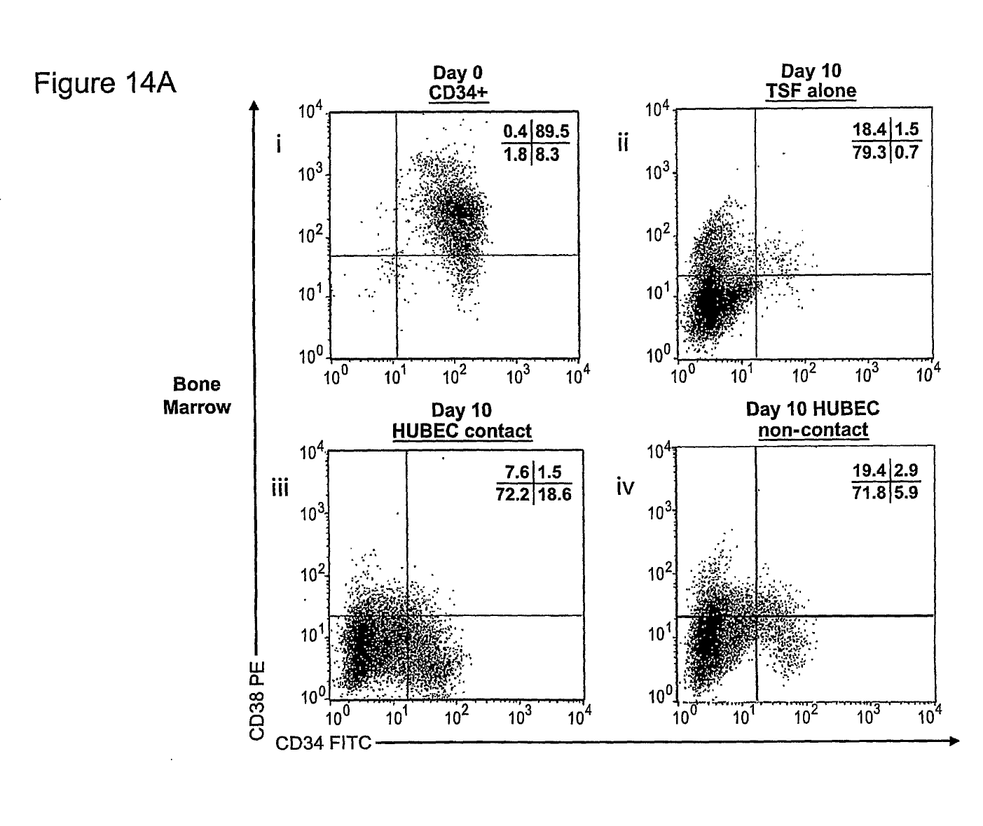

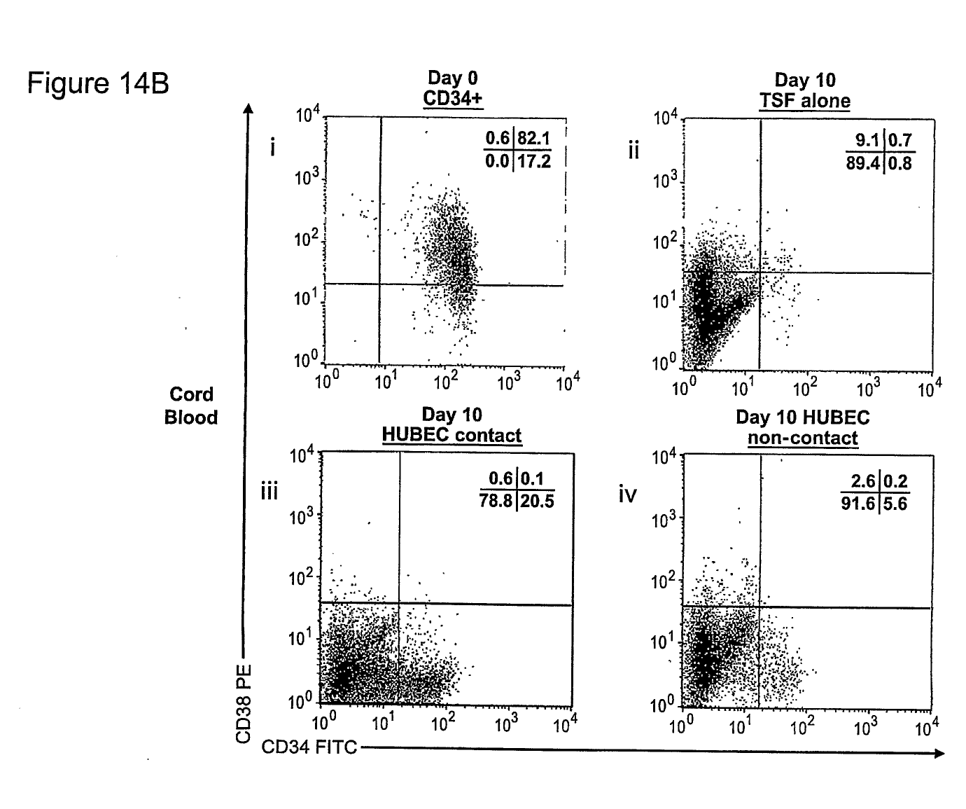

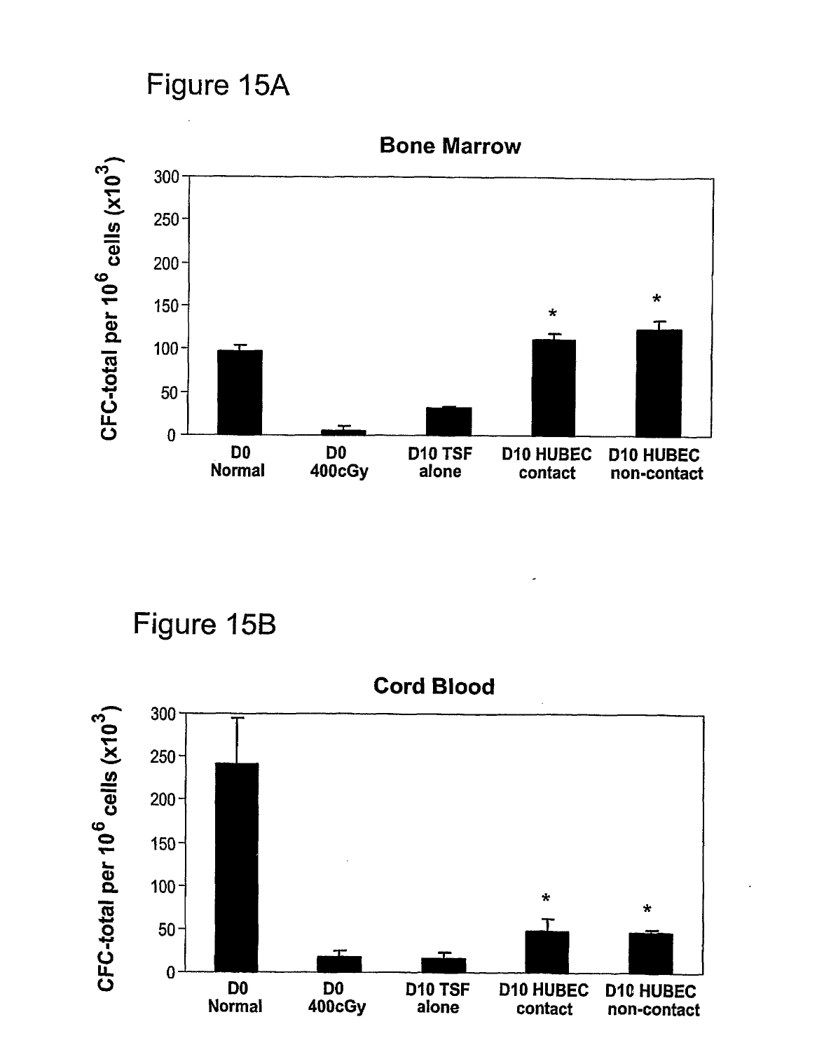

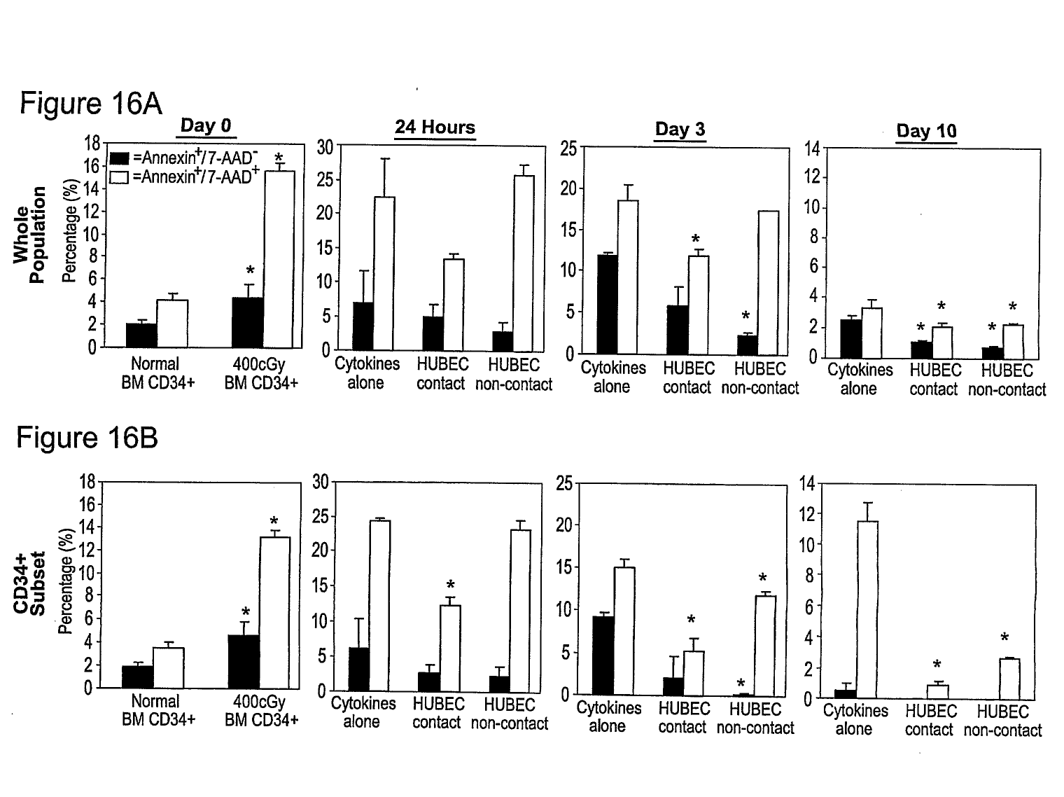

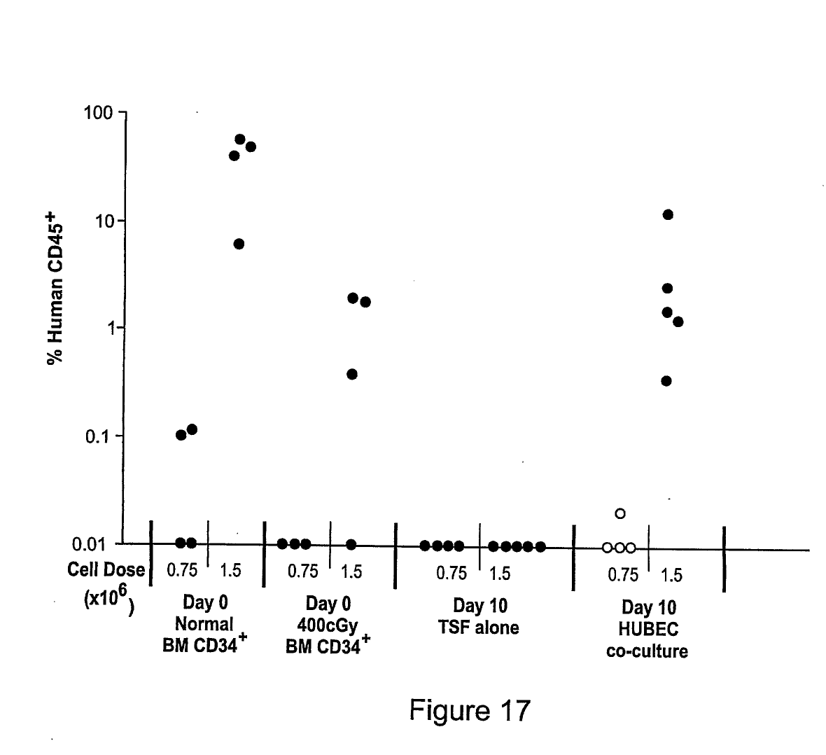

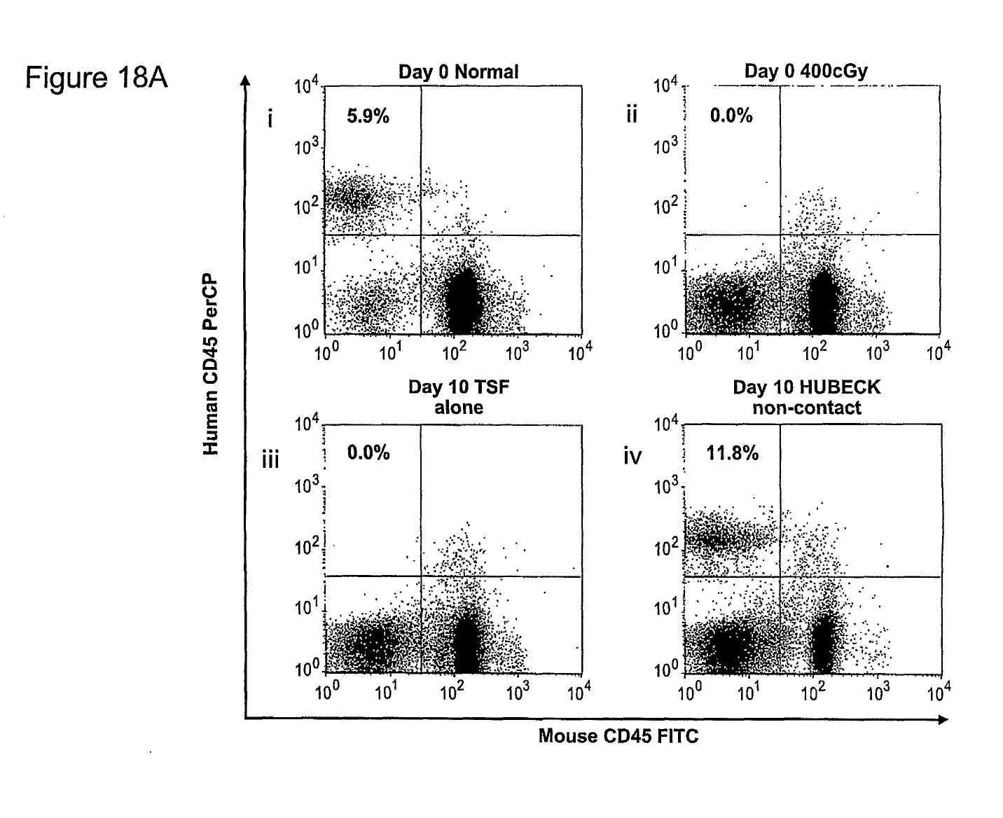

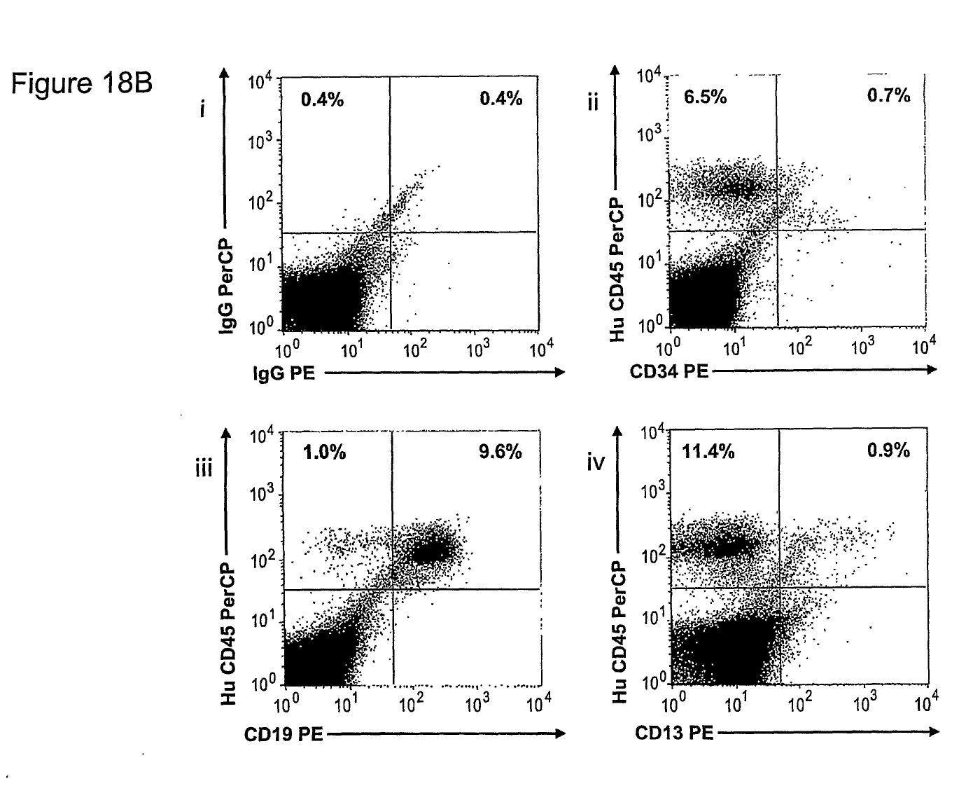

To extend our previous observations that primary HUBECs uniquely induce the expansion of human HSCs [13, 40, 41], we compared the in vitro expansion and in vivo repopulating capacity of human CB CD34⁺ cells following extended (14-day) culture with an optimal cytokine combination, thrombopoietin, stem cell factor, and Flt-3 ligand (TSF), versus transwell (non-contact) HUBEC cultures supplemented with TSF. As shown in FIG. 8A, 8B, total hematopoietic cell and CD34⁺ cell expansion at day 14 was significantly increased in the noncontact HUBEC cultures compared with TSF alone (p=0.008 and p=0.001, respectively; t test). More importantly, the progeny of 14-day noncontact cultures with HUBECs+TSF contained approximately eight fold increased numbers of SRCs compared with both input CD34⁺ cells and TSF-cultured cells (1 SRC in 8,200 cells [confidence interval: 1/3,800 to 1/19,000] versus 1 in 64,000 cells [1/14,000to 1/1,140,000], respectively; p=0.01, likelihood estimator model; FIG. 8C). These studies demonstrate the distinctly soluble hematopoietic activity elaborated by primary HUBECs and indicate its synergistic effect on SRC expansion when combined with thrombopoietin, stem cell factor (SCF), and Flt-3 ligand.

### Gene Expression Analysis Identifies Unique HUBEC Transcripts

To identify with a high degree of certainty the novel HUBEC-derived factors involved in HSC regulation, we applied a repetitive cDNA microarray analysis using Affymetrix human 133A and 133B chips, representing >47,000 annotated human genes. Highly purified RNA was isolated from biological replicates of primary HUBECs (n=5) and HUVECs (n=4) at 72 hours of culture and provided for array hybridization. Transcript lists generated from each sample were collected and analyzed following the MGED/MIAMI guidelines and subsequently reannotated into 26,570 nonredundant Unigene identifiers. To statistically identify the genes that were differentially expressed between HUBECs and HUVECs, a total of 4,477,203 probes in 18 hybridizations were fitted by using gene-by-gene ANOVA models. Volcano plot analysis revealed a highly consistent and nonredundant list of genes that were differentially expressed between HUBECs and HUVECs (FIG. 9)

ANOVA identified 65 nonredundant transcripts that were most consistently and highly over- or underexpressed within HUBECs. The minimum fold change of these genes was >2-fold, and the p value for each gene, corrected by the Bonferroni method, was <0.01. FIG. 10 is a colorimetric plot demonstrating the differential expression of these genes within the HUBEC and HUVEC sample sets. As anticipated, subtraction of the HUBEC transcriptome against that of HUVECs eliminated many housekeeping endothelial cell genes that we hypothesized were unlikely to play a role in HSC regeneration. In addition, this analysis revealed that primary HUBECs do not differentially express many established hematopoietic growth factors, including granulocyte colony stimulating factor, Flt-3 ligand, stem cell factor, thrombopoietin, interleukin (IL)-1, and IL-3. Table 6 shows the fold enrichment for various gene ontology categories within the top 65 transcripts. Fold enrichment was calculated by comparing each gene ontology category in the 65-gene set against all the genes on the chip. When organized by biological process, molecules involved in cell growth or the regulation of cell growth were >8-fold enriched within the top 65 transcripts, and 25 of the 65 genes (38%) were annotated to have a cell communication function (Table 1). Transcripts annotated to have extracellular location, extracellular activity, cell growth activity, and collagen structure were significantly enriched (p<0.001) within the genes upregulated in HUBECs. Conversely, transcripts annotated for function in cell adhesion and proteins integral to the membrane were significantly enriched (p<0.001) within the most downregulated genes within HUBECs. The Unigene identifiers and fold changes for each of the top 65 up- or downregulated HUBEC transcripts, along with their common gene names, are shown in Table 2. Fifteen of the 32 (47%) upregulated transcripts have extracellular activity or secreted protein properties, consistent with the soluble hematopoietic activity detected in our functional studies. As shown in Table 2, certain gene families were overrepresented within HUBECs, including insulin-like growth factor binding proteins (2 and 3), collagens (type I α2, IV α1, and VI α3), bone morphogenetic protein (BMP) antagonists (gremlin I homolog and follistatin), and stanniocalcins (1 and 2). The established interactions of these genes in other biological systems, such as folliculogenesis (IGFBPs and follistatin) [53], suggest that these overrepresented genes within HUBECs may participate in a coordinated process. Interestingly, none of the most upregulated transcripts within HUBECs are known to have definitive function in hematopoiesis or HSC self-renewal. Conversely, cell adhesion molecules, including ICAM 2, protocadherin, VE cadherin, and PECAM (CD31), were significantly down-regulated within HUBECs compared with HUVECs (Table 2). Using less stringent fold change-only criteria, we extended our analysis to include all transcripts with cell-cell signaling activity, hormone activity, and extracellular location that were >1.5-fold increased within HUBECs. These molecules are shown in supplemental online Table 6. The raw data from the complete HUBEC gene expression studies (Affymetrix CEL files) and all analyzed data (ratio of all genes) can be accessed directly at the Duke Bioinformatics Shared Resource web site (http://dbsr.duke.edu/pub/hubec). This web site provides accessible links to allow investigators to readily examine the complete HUBEC database.

TABLE 6
Gene ontology categories of top 65 transcripts

| | No. of genes | Fold | |
|---|---|---|---|
| Category | in top 65 | enrichment | p value |
| Cellular component | | | |
| Extracellular | 17 | 3.86 | <.001^{a} |
| Extracellular matrix | 7 | 5.80 | .001^{a} |
| Extracellular space | 8 | 5.10 | .010^{a} |
| Collagen | 4 | 26.40 | <.001^{a} |
| Integral to | 19 | 1.41 | .077^{b} |
| membrane | | | |
| Biological process | | | |
| Cell communication | 25 | 1.96 | <.001 |
| Cell-cell signaling | 5 | 2.08 | .206 |
| Cell-cell adhesion | 5 | 4.93 | .017^{b} |
| Cell adhesion | 11 | 4.31 | <.001^{b} |
| Organogenesis | 8 | 2.92 | .062 |
| Development | 14 | 2.05 | .011 |
| Regulation of cell | 3 | 8.29 | .049^{a} |
| growth | | | |
| Cell growth | 4 | 8.04 | .013^{a} |
| Molecular function | | | |
| Hormone activity | 3 | 6.52 | .075 |
| Binding | 34 | 1.11 | .233 |
| Extracellular matrix | 4 | 10.10 | .007 |
| structural | | | |
| constitutent | | | |

^{a}Fold enrichment for these gene ontology categories was most significant within the transcripts that were overexpressed by human brain endothelial cells (HUBECs) (p < .001 for each category).
^{b}Fold enrichment for these gene ontology categories was most significant within the transcripts that were underexpressed by HUBECs (p < .001).

The murine fetal liver stromal cell line, AFT024, has been shown to support the ex vivo maintenance of murine and human HSCs in cell-to-cell contact cultures [34, 54] The molecular profile of the AFT024 cell line has recently been published [54]. We hypothesized that common transcripts between HUBECs and AFT024 might represent an informatically validated list of HSC regulatory molecules. We interrogated the public StroCDB database (http://stromalcell.princeton.edu) [54] against the full-length sequences of all overrepresented transcripts within HUBECs, as shown in Table 7. Whereas the majority of the upregulated HUBEC transcripts failed to match with genes within the AFT024 transcriptome, 7 of the 32 sequences (22%) were found to be exact sequence matches (Table 8), including the soluble proteins IGFBP3, pregnancy-associated plasma protein A, autotaxin, and phosphodiesterase Ia. Autotaxin is of particular interest since this is a secreted phosphodiesterase that inhibits the cell adhesion of normal and malignant cells and promotes their motility [55]. Autotaxin and phosphodiesterase 1a fall within the same family of phospholipases, suggesting that the action of these phospholipases on target HSCs may contribute independently to their maintenance in vitro.

### Validation of Differential Expression of HUBEC-Specific Transcripts

To validate the results of the gene array analyses, quantitative real-time reverse transcription (RT)-PCR was performed for several genes identified to be overexpressed by HUBECs compared with HUVECs. Table 4 shows the expression of each gene within HUBECs and HUVECs relative to GAPDH control. IGFBP2, myocardin, and decorin were expressed in HUBECs but were below the level of detection within HUVECs, whereas adrenomedullin was 40-fold greater within HUBECs than HUVECs. These results demonstrated a good correlation between “present” and “absent” determinations within the gene array datasets and measurements of transcription by quantitative real-time PCR.

TABLE 7
Genes most overrepresented and underrepresented within human brain endothelial cells

| | | | | |
|---|---|---|---|---|
| | Fold | | | |
| Unigene ID | change | t score | Abbreviation | Gene name |

Overrepresented

| | | | | |
|---|---|---|---|---|
| Hs.433326 | 22.7 | 28.4697 | IGFBP2 | Insulin-like growth factor binding protein 2 |
| Hs.232115 | 18.4 | 41.001 | COL1A2 | Collagen, type I, α2 |
| Hs.421496 | 16.8 | 49.4707 | TGFBI | Transforming growth factor, β-induced |
| AF130082 | 15.9 | 27.6196 | | Unknown |
| Hs.23719 | 14.3 | 40.1201 | ENPP2 | Ectonucleotide pyrophosphatase/phosphodiesterase 2 |
| Hs.348037 | 14.0 | 29.6537 | PPP1R14A | Protein phosphatase I, regulatory subunit 14A |
| Hs.40098 | 13.8 | 44.7151 | GREM1 | Gremlin 1 homolog, cystein knot superfamily |
| Hs.160786 | 13.7 | 34.4033 | ASS | Arginosuccinate synthetase |
| Hs.433814 | 12.6 | 29.1636 | MYL9 | Myosin, light polypeptide 9 |
| Hs.233240 | 11.0 | 26.4445 | COL6A3 | collagen, type VI, α3 |
| Hs.2132 | 6.9 | 25.1668 | EPS8 | Epidermal growth factor receptor pathway substrate 8 |
| Hs.450230 | 6.9 | 39.3367 | IGFBP3 | Insulin-like growth factor binding protein 3 |
| Hs.56 | 6.3 | 34.9096 | PRPS1 | Phosphoribosyl pyrophosphate synthetase 1 |
| Hs.437173 | 6.1 | 35.7357 | COL4A1 | Collagen, type IV, α1 |
| Hs.42128 | 6.1 | 28.6323 | MYOCD | Myocardin |
| Hs.407912 | 5.5 | 34.2239 | COL4A2 | Collagen, type IV, α2 |
| Hs.356509 | 5.0 | 37.9042 | CXXC5 | CXXC finger 5 |
| Hs.155223 | 4.8 | 32.703 | STC2 | Stanniocalcin 2 |
| Hs.356289 | 3.9 | 33.1474 | URB | Steroid-sensitive gene 1 |
| Hs.9914 | 3.7 | 30.1854 | FST | Follistatin |
| Hs.188757 | 3.4 | 26.7281 | C6orf69 | Chromosome 6 open reading frame 69 |
| Hs.194431 | 3.2 | 31.4286 | KIAA0992 | Palladin |
| Hs.25590 | 3.2 | 25.9193 | STC1 | Stanniocalcin 1 |
| Hs.296398 | 3.2 | 31.9694 | LAPTM4B | Lysosomal-associated protein transmembrane 4β |
| Hs.10862 | 2.9 | 25.3457 | AK3 | Adenylate kinase 3 |
| Hs.440769 | 2.7 | 29.8539 | PAPPA | Pregnancy-associated plasma protein A |
| Hs.31297 | 2.6 | 26.2109 | CYBRD1 | Cytochrome b reductase 1 |
| Hs.199695 | 2.4 | 26.7861 | MAC30 | Hypothetical protein MAC30 |
| Hs.21486 | 2.3 | 56.1385 | STAT1 | Signal transducer and activator of transcription 1 |
| Hs.421383 | 2.3 | 25.5939 | FHL1 | Four and a half LIM domains 1 |
| Hs.416061 | 2.3 | 28.4021 | PDE1A | Phosphodiesterase 1A, calmodulin-dependent |
| Hs.197081 | 2.3 | 25.4848 | ADAP12 | A kinase (PRKA) anchor protein 12 |

Underrepresented

| | | | | |
|---|---|---|---|---|
| Hs.433303 | −17.2 | −44.82385 | ICAM2 | Intracellular adhesion molecule 2 |
| Hs.495628 | −13.6 | −42.49424 | C10orf58 | Chromosome 10 open reading frame 58 |
| Hs.78224 | −13.1 | −29.80438 | RNASE1 | RNase A family, 1 |
| Hs.146858 | −12.2 | −32.5373 | PCDH10 | Protocadherin 10 |
| Hs.279575 | −10.5 | −28.68902 | GPR91 | G protein-coupled receptor 91 |
| Hs.124491 | −9.8 | −38.03536 | LXN | Latexin |
| Hs.413504 | −9.7 | −37.87727 | HHIP | Hedgehog interacting protein |
| Hs.185055 | −9.7 | −31.10791 | BENE | BENE protein |
| Hs.84072 | −9.5 | −27.58264 | TM4SF3 | Transmembrane 4 superfamily member 3 |
| Hs.84072 | −9.3 | −58.46405 | EMCN | Endomucin |
| Hs.76206 | −9.0 | −26.11165 | CDH5 | Cadherin 5, type 2, VE-cadherin |
| Hs.78824 | −7.9 | −29.80102 | TIE | Tyrosine kinase with Ig and E.G.F. homology domains |
| Hs.483538 | −7.6 | −43.29464 | | Transcribed sequence with strong similarity to protein sp: P00722 |
| Hs.301175 | −5.9 | −27.84813 | RAC2 | ras-related C3 botulinum toxin substrate 2 |
| Hs.387579 | −5.9 | −26.16374 | CD9 | CD9 antigen |
| Hs.511397 | −5.4 | −29.71115 | MCAM | Melanoma cell adhesion molecule |
| Hs.81008 | −5.1 | −38.824 | FLNB | Filamin B, β |
| Hs.243010 | −4.9 | −49.26564 | RHOJ | ras homolog gene family, member J |
| Hs.511899 | −4.4 | −28.97667 | EDN1 | Endothelin 1 |
| Hs.301989 | −4.2 | −29.48633 | STAB1 | Stabilin 1 |
| Hs.151155 | −3.9 | −25.35221 | TM6SF1 | Transmembrane 6 superfamily member 1 |
| Hs.78146 | −3.7 | −30.43546 | PECAM1 | Platelet/endothelial cell adhesion molecule (CD31) |
| Hs.105468 | −3.6 | −27.41569 | hIAN2 | Human immune-associated nucleotide 2 |
| Hs.409602 | −3.6 | −30.89745 | SULF1 | Sulfatase 1 |
| Hs.75514 | −3.4 | −25.9131 | NP | Nucleoside phosphorylase |
| Hs.257222 | −3.3 | −25.95915 | B3GNT5 | UDP-GlcNAc:βGalβ-1,3-N-acetylglucosaminyltransferase 5 |
| Hs.26612 | −2.6 | −26.60272 | PGM2L1 | Phosphoglucomutase 2-like 1 |
| Hs.277324 | −2.6 | −27.06172 | GALNT | N-Acetylgalactosaminyltransferase 1 |
| Hs.5897 | −2.4 | −26.73473 | ANTXR2 | Anthrax toxin receptor 2 |
| Hs.434488 | −2.4 | −28.36924 | CSPG2 | Chondroitin sulfate proteoglycan 2 |
| Hs.279518 | −2.3 | −31.28838 | APLP2 | Amyloid β(A4) precursor-like protein |

TABLE 8
Common transcripts between HUBEC and AFT024

| Unigene ID | Gene name | E value |
|---|---|---|
| Hs.232115 | Collagen type I, α2 | <1 × 10⁻⁵ |
| Hs.23719 | ENPP2 (Autotaxin) | <1 × 10⁻⁵ |
| Hs.450230 | IGFBP3 | 4 × 10⁻²⁹ |
| Hs.443625 | Collagen type VI, α3 | 1 × 10⁻⁶⁶ |
| Hs.440769 | Pregnancy-associated protein | 4 × 10⁻⁷² |
| Hs.356289 | Steroid-senstive gene 1 (URB) | 2 × 10⁻⁹⁶ |
| Hs.416061 | PDE1A | 2 × 10⁻¹⁴ |

A Blast query search was performed to identify transcripts in common between primary HUBEC and the murine fetal liver stromal cell line, AFT024 [57]. Using the public StroCDB database search engine, the entire gene sequences of the top 32 HUBEC transcripts were queried against the AFT024 transcriptome at http://www.stromalcell.princeton.edu. Gene sequences were considered matched if the E value for the homology was ≦1 × 10⁻⁵.
Abbreviation:
HUBEC, human brain endothelial cell.

TABLE 9
Quantitative real-time RT-PCR analysis of
representative genes

| Gene | HUBECs | HUVECs |
|---|---|---|
| GAPDH | 1.0 ± 0.08 | 1.0 ± 0.03 |
| IGFBP2 | 0.11 ± 0.01 | Not detectable |
| Decorin | 0.23 ± 0.02 | Not detectable |
| Adrenomedullin | 0.08 ± 0.01 | 0.002 ± 0.001 |
| Myocardin | 0.04 ± 0.03 | Not detectable |

Highly purified total RNA (2 μg per sample) was isolated from primary HUBECs and HUVECs and reverse-transcribed as described in Materials and Methods. Fifty-nanogram equivalents of cDNA were then used for quantitative real-time PCR for decorin, insulin-like growth factor binding protein 2 (IGFBP2), myocardin, adrenomedullin, and glyceraldehyde-3-phosphate dehydrogenase (GAPDH). Relative gene expression between HUBECs and HUVECs was calculated using the ΔΔCt method, using GAPDH expression as a normalization reference.
Abbreviations:
GAPDH, glyceraldehyde-3-phosphate dehydrogenase;
HUBEC, human brain endothelial cell;
HUVEC, human umbilical vein endothelial cell;
IGFBP2, insulin-like growth factor binding protein 2.

### Functional Assay of HUBEC-Derived Soluble Proteins

To begin to define the hematopoietic capacity of the novel proteins produced by HUBECs, we first assayed the activity of four HUBEC-derived proteins against primary human CB CD34⁺ cells based upon their fold upregulation (IGFBP2 and IGFBP3), their annotated soluble or extracellular activity (adrenomedullin and follistatin), and their collective lack of defined hematopoietic activity. As shown in FIG. 11 either IGFBP2, IGFBP3, nor follistatin demonstrated any additive hematopoietic effect with regard to total cell or CD34⁺ cell expansion when combined with TSF. However, the addition of 50-100 ng/ml adrenomedullin to TSF caused a significant increase in total cell and CD34⁺ cell expansion compared with TSF alone (p=0.001 and p=0.002, respectively), suggesting a potentially direct effect of adrenomedullin on human hematopoietic progenitor cells. Moreover, when we assayed HSC-enriched CD34⁺CD38⁻lin⁻ cells alone, the addition of 100 ng/ml adrenomedullin significantly increased the proliferation of this primitive population when combined with SCF or Flt-3 ligand, as compared with either cytokine alone (p=0.01 and p=0.003; respectively; FIG. 12. Again, neither IGFBP2 nor follistatin supported an additive effect upon SCF, Flt-3 ligand, or TSF. However, IGFBP3 at 50 ng/ml was associated with an increase in the proliferation of CD34⁺CD38⁻lin⁻ cells in combination with Flt-3 ligand as compared with Flt-3 ligand alone (p=0.01). Taken together, these data suggested that IGFBP3 and, in particular, adrenomedullin, are candidate endothelial cell-derived growth factors with hematopoietic activity.

### Discussion

One strategy that has been employed to characterize the biology of HSCs has involved the molecular analysis of purified cell populations enriched for HSCs compared with committed progenitors. Ivanova et al. examined the gene expression profile of murine BM Lin⁻c-kit⁺ Sca-1⁺Rho^{low} cells (HSCs) versus murine fetal liver HSC, human fetal liver HSC, embryonic neural stem cells, and an embryonic stern cell line and found 283 transcripts enriched within all three stem cell populations [56]. Ramahlo-Santos et al. [57] similarly identified 216 transcripts that were enriched within murine HSC, neural stem cells, and embryonic stem cells. Interestingly, comparison of the lists of stem cell-associated genes from the two studies revealed only six genes in common between the two [58]. An additional analysis by Georgantas et al. comparing the unique genes within the transcriptome of human CD34⁺CD38⁻ lin⁻ cells versus the reported findings of overrepresented genes within three other data sets of purified murine and human HSC populations determined that only one gene, the GATA3 transcription factor, was common to all data sets [58]. One explanation for the lack of commonalities between these studies may be the inherent limitations in methods to isolate pure HSC populations in the absence of contaminating cells, as well as the different methods used to isolate stem cell populations across these studies. In this study, we chose to examine the transcriptional profile of a homogeneous population of primary human endothelial cells that support the ex vivo expansion of human HSCs [40, 41]. Molecular analyses of effector cells that support the maintenance or expansion of HSCs have been much less frequently reported [54], particularly due to a lack of effector cells capable of inducing HSC expansion. The approach we have taken offers the benefit of identifying human genes that have a likelihood of direct Involvement in signaling the maintenance and expansion of human HSCs. Concordantly, we are pursuing studies to determine whether the conditioned medium alone from HUBECs is capable of inducing HSC expansion, as well as the identification of HUBEC-secreted proteins via high-throughput chromatographic separation.

Our analysis identified 65 genes that were significantly over- or underexpressed within HUBECs compared with HUVECs, with consistency across multiple biological replicates. Gene ontology studies demonstrated that the majority of the upregulated genes within HUBECs were extracellular and/or involved in triggering cell growth. The identification of trans-forming growth factor-β-induced protein as one of the most highly overexpressed HUBEC gene products is noteworthy in light of the established function of transforming growth factor-p in inhibiting HSC cycling and proliferation [59]. Many of the overexpressed transcripts include families of genes, such as IGFBPs, which regulate mesodermal cell fate decisions. IGFBP2 has been shown to inhibit embryonic fibroblast proliferation and can induce growth arrest of type II alveolar epithelial stem cells [60, 61]. IGFBP3 inhibits the proliferation of mesenchymal progenitors and fibroblasts in an IGF-1-independent manner [62]. IGFBP3 levels have also been positively associated with effective erythropoiesis in children, suggesting a potential physiologic role for this secreted protein in hematopoiesis [63]. Collagen family subtypes, specifically collagen type I α2 and collagen type IV α1, were also significantly overrepresented within HUBECs. Although the adhesion of HSCs to extracellular matrix molecules, such as fibronectin and collagen type I [64], has been associated with short-term maintenance of repopulating cells, the soluble hematopoietic activity of collagen moieties has not been demonstrated. Interestingly, adiponectin, which is a member of the family of soluble defense collagens, has recently been shown to inhibit colony-forming cell activity in suspension cultures [65], raising the possibility that collagen moieties produced by HUBECs may contribute to the soluble hematopoietic activity we have observed.

The upregulation of two BMP antagonists, follistatin and gremlin 1 homolog, was somewhat surprising in light of the previously demonstrated contribution of BMP signaling in embryonic hematopoiesis [66]. Follistatin is an inhibitor of follicle-stimulating hormone and activin [67] and causes lethality in knockout mice via failure of brain, lung, and soft tissue development at day 15.5 [68]. A potential role for the activin/follistatin pathway in hematopoiesis has been implied, but not confirmed, by studies indicating that activin exposure promoted red blood cell differentiation in mice [69]. Gremlin 1 homolog, like follistatin, also inhibits the activity of BMPs, specifically BMP2, BMP4, and BMP 7. Interestingly, a gremlin 1 null mutation in the mouse induces neonatal lethality secondary to failure of nephric and lung organ development, and exogenous gremlin 1 has been shown to have antiapoptotic effects on mesodermal cells in vitro [70]. The direct role for gremlin 1 and follistatin in hematopoiesis has yet to be demonstrated, but given that these molecules inhibit progenitor cell differentiation in other organ systems [71], it is plausible that either might inhibit differentiation of proliferating HSCs.

Two of the most highly overexpressed transcripts within HUBECs, stanniocalcin 1 and 2, regulate calcium/phosphorus homeostasis in fish and humans and induce proliferation and differentiation of osteoblasts in vitro. [72]. URB (steroid-sensitive gene 1) is a 150-10a secreted protein and was recently characterized in the mouse to have a role in skeletogenesis [73]. In light of the recent demonstration of the osteoblastic niche for HSCs in the bone marrow, these data suggest the possibility that hormones elaborated by endothelial cells, possibly brain endothelial cells, may regulate osteoblast activity in the BM. A neuroendocrine-hematopoietic axis has been postulated previously [74], and the enrichment for osteoblast-regulatory factors within HUBECs further suggests this possibility. The recent demonstration of overlapping genetic programs between neural and hematopoietic stem cells [75] also suggests that brain-derived factors may have hematopoietic activity.

Of additional interest was the examination of transcripts that were downregulated in HUBECs compared with HUVECs. Of note, protocadherin and VE-cadherin were markedly underexpressed in HUBECs compared with HUVECs. Since cadherin-based interactions have recently been implicated in the contact-dependent maintenance of quiescent HSCs in vivo within the osteoblastic marrow niche [24], this implies that such interactions might be important for ex vivo maintenance of HSCs in culture. Despite this, noncontact HUBEC cultures and the results of this gene expression analysis indicate that the cadherin-based contact interactions are not important for expansion of HSCs in the HUBEC culture system. Taken together, these data implicate a novel soluble factor or factors elaborated by HUBECs that promote the expansion of human repopulating cells. Moreover, these data suggest that the interaction of HSCs with cadherin moities may inhibit the proliferation of HSCs, thereby maintaining quiescence. Further studies will be important to delineate differences in the cell cycle status and SCID-repopulating capacity of HSCs cultured with osteoblasts and those cultured under noncontact conditions with HUBECs.

The fetal liver murine stromal cell line AFT024 has been shown, in contact cultures, to support the ex vivo maintenance of murine and human HSCs [34, 35]. In contrast to HUBECs, which support HSC expansion equally under contact or noncontact conditions [43, 44], AFT024 support of LTC-IC has been shown to decline under noncontact conditions [76]. When we queried the most upregulated HUBEC transcripts against the AFT024 database, we identified seven transcripts in common between HUBECs and AFT024, including collagen type I and VI, IGFBP3, URB, and autotaxin. We have prioritized functional assay of these genes via loss of function small inhibitory RNA studies, since these molecules should have a high probability of participation in HSC signaling. We also anticipate that other extracellular HUBEC transcripts unique from the AFT024 transcriptome will prove to be functionally important in HSC regulation in light of the distinctly soluble nature of the HUBEC hematopoietic activity that we have observed.

As an initial strategy to screen for the hematopoietic activity of novel growth factors expressed by HUBECs, we have analyzed a group of proteins that are available in recombinant form and have established extracellular function: IGFBP2, IGFBP3, follistatin, and adrenomedullin. Interestingly, one of these proteins, adrenomedullin, augments the expansion of human CD34⁺ cell when combined with thrombopoietin, SCF, and Flt-3 ligand, while also enhancing the individual activities of SCF and Flt-3 ligand on HSC-enriched CD34⁺CD38⁻lin⁻ cells in vitro. These data indicate that further studies are merited to define the effects of adrenomedullin on HSC fate and hematopoiesis in general, in addition to our planned siRNA gene silenCing studies to determine the precise contribution of adrenomedullin to HUBEC-mediated HSC expansion. Although it has been established that adrenomedullin is required for normal cardiovascular development [77], the hematopoietic activity of adrenomedullin has not been well characterized. However, human CD34⁺ cells express the calcitonin receptor-like receptor, the receptor for adrenomedullin [78], and stromal cells expressing adrenomedullin as well as other growth factors support human colony-forming cell growth in vitro [79]. We plan to produce and functionally assay each of the genes with secretory or extracellular domains that are overexpressed by HUBECs and anticipate that the reproduction of HUBEC stem cell-supportive activity may require the combination of several proteins identified thus far.

In summary, we have presented a molecular profile of novel endothelial cells that support the ex vivo expansion of human HSCs. Since HUBECs are unlike other established stromal cell lines (e.g., AFT024) in the soluble nature of their HSC-supportive activity, it is plausible that novel soluble proteins produced by HUBECs can be identified and characterized. The identities of these factors may overlap with secreted factors produced within the BM microenvironment that support in vivo HSC maintenance and proliferation [1, 22-24]. The HUBEC molecular profile is a template for the identification of soluble factors that mediate hematopoietic stem cell fate.

### REFERENCES

1 Sorrentino P. Clinical strategies for expansion of haematopoietic stem cells. Nat Rev Immunol 2004; 4:878-888.
2 Osawa M, Hanada K, Hamada H, Nakauchi H. Long-term lymphohematopoietic reconstitution from a single CD34−/low hematopoietic stem cell. Science 1996; 273:242-245.
3 Cashman J et al. Kinetic evidence of the regeneration of multilineage hematopoiesis from primitive cells in normal human bone marrow transplanted into immune deficient mice. Blood 1997; 80:4307-4316.
4 Rocha V, Cornish J, Sievers E et al. Comparison of outcomes of unrelated bone marrow and umbilical cord blood transplants in children with acute leukemia. Blood 2001; 97:2962-2971.
5 Storb R, Blume K, O′Donnell M et al. Cyclophosphamide and anti-thymocyte globulin to condition patients with aplastic anemia for allogeneic marrow transplantations: The experience of 4 centers. Biol Blood Marrow Transplant 2001; 7:39-44.
6 Kiehl M, Kraut L, Schwerdtfeger T et al. Outcome in hematopoietic stem cell transplantation in adult patients with acute lymphoblastic leukemia: No difference in related compared with unrelated transplant in first complete remission. J Clin Oncol 2004; 15:2816-2825.
7 Gur H, Krauthgamer R, Berrebi A et al. Tolerance induction by megadose hematopoietic progenitor cells: Expansion of veto cells by short term culture of purified human CD34⁺ cells. Blood 2002; 99: 4174-4181.
8 Cogle C, Weinman D, Jorgensen M et al. Adult human hematopoietic cells provide functional hemangioblast activity. Blood 2004; 103:133-135.
9 Rafit S, Lyden D. Therapeutic stem and progenitor cell transplantation for organ vascularization and regeneration. Nat Med 2003; 9:702-712.
10 Bhatia M, Wang J, Kapp U et al. Purification of primitive human hematopoietic cells capable of repopulating immune-deficient mice. Proc Natl Acad Sci USA 1997; 94:5320-5325.
11 Krosl J, Austin P, Beslu N et al. In vitro expansion of hematopoietic stem cells by recombinant TAT-HOXB4 protein. Nat Med 2003; 9:1428-1432.
12 Piacibello W, Bruno S, Sanavio F et al. Lentiviral gene transfer and ex vivo expansion of human primitive stem cells capable of primary, secondary, and tertiary multilineage repopulation in NOD/SCID mice. Blood 2002; 15:4391-4400.
13 Chute J, Saini A A, Chute D J et al. Co-culture with human brain endothelial cells increases the SCID repopulating capacity of adult human bone marrow. Blood 2002; 100:4433-4439.
14 Kawano Y, Kobune M, Yamaguchi M et al. Ex vivo expansion of human umbilical cord hematopoietic progenitor cells using a coculture system with human telomerase catalytic subunit (hTERT) transfected human stromal cells. Blood 2003; 101:532-540.
15 Reya T, Duncan A, Allies L et al. A role for Wnt signalling in the self renewal of hematopoietic stem cells. Nature 2003; 423:409-414.
16 Vamum-Finney B, Xu L, Brashem-Stein C et al. Pluripotent, cytokine-dependent, hematopoietic stem cells are immortalized by constitutive Notch1 signaling. Nat Med 2000; 6:1278-1281.
17 Bhardwaj G, Murdoch B, Wu D at al. Sonic hedgehog induces the proliferation of primitive human hematopoietic cells via BMP regulation. Nat Immunol 2001; 2:172-180.
18 Amsellem S, Pflumio F, Bardinet D et al. Ex vivo expansion of human hematopoietic stem cells by direct delivery of the HOXB4 homeoprotein. Nat Med 2003; 91423-1427.
19 Basser R, To L, Begley C et al. Rapid hematopoietic recovery after multicycle high dose chemotherapy: Enhancement of filgrastim-induced progenitor cell mobilization by recombinant human stem cell factor. J Clin Oncol 1998; 16:1899-1908.
20 Rizzo J, Lichtin A, Woolf S et al. Use of epoietin in patients with cancer: Evidenced based clinical practice guidelines of the American Society of Clinical Oncology and the American Society of Hematology. J Clin Oncol 2002; 20:4083-4107.
21 Bennett C, Smith T, Weeks J et al. Use of hematopoietic colony stimulating factors: The American Society of Clinical Oncology survey. The Health Services Research Committee of the American Society of Clinical Oncology. J Clin Oncol 1996; 14:2511-2520.
22 Avecilla S, Hattori K, Heissig B at al. Chemokine mediated interaction of hematopoietic progenitors with the bone marrow vascular niche is required for thrombopoiesis. Nat Med 2004; 10:64-71.
23 Calvi L, Adams G, Weibrecht K et al. Osteoblastic cells regulated the haematopoietic stem cell niche. Nature 2003; 425:841-846.
24 Zhang 0.1, Niu C, Ye Let al. Identification of the haematopoietic stem cell niche and control of the niche size. Nature 2003; 425:836-841.
25 Shalaby F, Rossant J, Yamaguchi T P et al. Failure of blood island formation and vasculogenesis in flk-1 deficient mice. Nature 1995; 376: 62-66.
26 Oberlin E, Tavian M, Blazsek I et al. Blood-forming potential of vascular endothelium in the human embryo. Development 2002; 129: 4147-4157.
27 Choi K, Kennedy M, Kazarov A et al. A common precursor for hematopoietic and endothelial cells. Development 1998; 125:725-732.
28 Lu L, Wang S, Auerbach R. In vitro and in vivo differentiation into B cells, T cells, and myeloid cells of primitive yolk sac hematopoietic progenitor cells expanded >100 fold by a clonal yolk sac endothelial cell line. Proc Natl Acad Sci USA 1996; 93:14782-14787.
29 Rafii S, Shapiro F, Rimarachin J et al. Isolation and characterization of human bone marrow microvascular endothelial cells: Hematopoietic progenitor cell adhesion. Blood 1994; 84:10-19.
30 Rafii S, Shapiro F, Pettengell R et al. Human bone marrow microvascular endothelial cells support long term proliferation and differentiation of myeloid and megakaryocytic progenitors. Blood 1995; 86:3353-3363.
31 Tavian M, Coulombel L, Luton D et al. Aorta associated CD34⁺ hematopoietic cells in the early human embryo. Blood 1996; 87:67-72.
32 Heissig B, Hattori K. Dias S et al. Recruitment of stem and progenitor cells from the bone marrow niche requires MMP-9 mediated release of kit ligand. Cell 2002; 109:625-637.

33 Gan O, Murdoch B, Larochelle A et al. Differential maintenance of primitive SCID-repopulating cells, clonogenic progenitors, and long-term culture initiating cells after culture on human bone marrow stromal cells. Blood 1997; 90:641-650.
34 Punzel M, Liu D, Zhang T et al. The symmetry of initial divisions of human hematopoietic progenitors is altered only by the cellular microenvironment. Exp Hematol 2003; 31:339-347.
35 Lewis I, Almeida-Porada G, Du J et al. Umbilical cord blood cells capable of engrafting in primary, secondary, and tertiary xenogeneic hosts are preserved after ex vivo culture in a non-contact system. Blood 2001; 7:3441-3449.
36 Chute J P, Kampen R, Wells R et al. A comparative study of the cell cycle status and primitive cell adhesion molecule profile of human CD34⁺ cells cultured in stroma-free versus porcine microvascular endothelial cell cultures. Exp Hematol 1999; 27:370-379.
37 Chute J P, Saini A, Wells M et al. Pre-incubation with endothelial cell monolayers increases gene transfer efficiency into human bone marrow CD34⁺CD38⁻ progenitor cells. Hu Gene Ther 2000; 11:2515-2528.
38 Chute J, Fung J, Muramoto G et al. Ex vivo culture rescues hematopoietic stem cells with long term repopulating capacity following harvest from lethally irradiated mice. Exp Hematol 2004; 32:308-317.
39 Brandt J E, Bartholemew A M, Fortman J D et al. Ex vivo expansion of autologous bone marrow stem cells with porcine microvascular endothelial cells results in a graft capable of rescuing lethally irradiated baboons. Blood 1999; 94; 106-113.
40 Chute J P, Fung J, Muramoto G, Oxford C. Quantitative analysis demonstrates expansion of SCID-repopulating cells (SRC) and increased engraftment capacity within human cord blood cells following culture with human brain endothelial cells. STEM CELLS 2004; 22:202-215.
41 Chute J P, Muramoto G, Fung J et al. Soluble factors elaborated by human brain endothelial cell induce the concomitant expansion of purified BM CD34⁺CD38⁻ cells and SCID repopulating cells. Blood 2005; 105:576-583.
42 Schulz L, Schweitzer P, Christianson S et al. Multiple defects in innate and adaptive immunologic function in NOD/LtSz-scid mice. J Immunol 1995; 154:180-191.
43 Taswell C. Limiting dilution assays for the determination of immunocompetent cell frequencies, I: Data analysis. J Immunol 1981; 126:1614-1619.
44 Wang J, Doedens M, Dick J. Primitive human hematopoietic cells are enriched in cord blood compared with adult bone marrow or mobilized peripheral blood as measured by the quantitative in vivo SCID-repopulating cell assay. Blood 1997; 89:3919-3924.
45 Jazwiec B, Solanilla A, Grosset C et al. Endothelial cell support of hematopoiesis is differentially altered by IL-1 and glucocorticoids. Leukemia 1998; 8:1210-1220.
46 Coombes B, Mahoney J. cDNA array analysis of altered gene expression in human endothelial cells in response to Chlamydia pneumoniae infection. Infect Immun 2001; 69:1420-1427.
47 Gerritsen M, Soriano R, Yang S at al. Branching out: A molecular fingerprint of endothelial differentiation into tube-like structures generated by Affymetrix oligonucleotide arrays. Microcirculation 2003; 10:63-81.
48 Soon L, Yie T, Shvarts A at al. Overexpression of WISP-1 downregulated motility and invasion of lung cancer cells through inhibition of Rae activation. J Biol Chem 1003:278:11465-11470.
49 Cousins R, Blanchard R, Popp M et al. A global view of the selectivity of Zinc deprivation and excess on genes expressed in human THP-1 mononuclear cells. Proc Natl Aced Sci USA 2003; 100:6952-6957.
50 Brazma A, Hingamp P, Quackenbush J at al. Minimum information about a microarray experiment (MIAME)-toward standards for microarray data. Nat Genet 2001; 29:365-371.
51 Dudoit S, Gentleman R, Quackenbush J. Open source software for the analysis of microarray data Biotechniques 2003; supp1:45-51.
52 Hosack D, Dennis G, Sherman B et al. Identifying biological themes within lists of genes with EASE. Genome Biol 2003; 4:R70.
53 Klein N, Battaglia D, Woodruff T et al. Ovarian follicular concentrations of activist, follistatin, inhibin, insulin-like growth factor I (IGF-1), IGF-IGF binding protein 2 (IGFBP2), IGFBP3, and vascular endothelial growth factor in spontaneous menstrual cycles of normal women of advanced reproductive age. J Clin Endocrinol Metab 2000; 85:4520-4525.
54 Hackney 1, Charboard P, Brunk B et al. A molecular profile of a hematopoietic stern cell niche. Proc Nail Acad Sci USA 2002; 99: 13061-13066.
55 Fox M, Colello R, Macklin W et al. Phosphodiesterase-1 alpha/autotaxin: A counteradhesive protein expressed by oligodendrocytes during onset of myelination. Mol Cell Neurosci 2003; 23:507-519.
56 Ivanova N, Dimos J, Schaniel C et al. A stern cell molecular signature. Science 2002; 298:601-604.
57 Ramalho-Santos M, Yoon S, Matsuzaki Y et al. “Sternness”: Transcriptional profiling of embryonic and adult stem cells. Science 2002; 298: 597-600.
58 Georgantas R, Tanavde V, Malehorn M et al. Microarray and serial analysis of gene expression analyses identify known and novel transcripts overexpressed in hematopoietic stem cells. Cancer Res 2004; 64: 4434-4441.
59 Sitnicka E, Ruscetti F, Priestley G et al. Transforming growth factor beta 1 directly and reversibly inhibits the initial cell divisions of long term repopulating hematopoietic stern cells. Blood 1996; 88:82-88.
60 Hofflich A, Lahm H, Blum W et al. Insulin like binding protein 2 inhibits proliferation of human embryonic kidney fibroblasts and of IGF responsive colon carcinoma cell lines. FEBS Lett 1998; 434:329-334.
61 Mouhieddine O, Cazals V, Kuto E et al. Glucocorticoid growth arrest of lung alveolar epithelial cells is associated with increased production of insulin like growth factor binding protein 2. Endocrinology 1996; 137: 287-295.
62 Longobardi L, Torello M, Buckway C et al. A novel insulin-like growth factor I independent role for insulin like growth factor binding protein 3 in mesenchymal chondroprogenitor cell apoptosis. Endocrinology 2003; 144:1695-1702.
63 Vihervouri E, Virtanen M, Koistinen H et al. Hemoglobin level is linked to growth hormone dependent proteins in short children. Blood 1996; 87: 2075-2081.
64 Bhatia R, Williams A, Munthe H. Contact with fibronectin enhances maintenance of normal but not chronic myelogenous leukemia primitive hematopoietic progenitors. Exp Hematol 2002; 30:324-332.
65 Yokota T, Oritani K, Takahashi I et al. Adiponectin, a new member of the family of soluble defense collagens, negatively regulates the growth of myelomonocytic progenitors and the functions of macrophages. Blood 2000; 96:1723-1732.
66 Zon L. Self renewal versus differentiation, a job for the mighty morphogens. Nat Immunol 2001; 2:142-143.
67 Meehan T, Schlatt S, O′Bryan M et al. Development of germ cell and Sertoli cell development by activin A, follistatin, and FSH. Dev Biol 2000; 220225-237.
68 Matzuk M, Lu N, Vogel H et al. Multiple defects and perinatal death in mice deficient in follistatin. 1995; 374:360-363.
69 Yamashita T, Takahashi S, Ogata E. Expression of activin/erythroid differentiation factor in murine bone marrow stromal cells. Blood 1992; 79:304-307.
70 Michos O, Panman L, Vintersten K et al. Gremlin-mediated BMP antagonism induces the epithelial-mesenchymal feedback signaling controlling metanephric kidney and limb organogenesis. Development 2004; 131:3401-3410.
71 Zhang Y, Cleary M, Si Y at al. Inhibition of activin signaling induces pancreatic epithelial cell expansion and diminishes terminal differentiation of pancreatic beta cells. Diabetes 2004; 53:2024-2033.
72 Yoshiko Y, Aubin J. Stanniocalcin 1 as a pleiotropic factor in mammals. Peptides 2004; 25:1663-1669.
73 Liu Y, Monticone M, Tonachïni L et al. URB expression in human bone marrow stromal cells and during mouse development. Biochem Biophys Res Commun 2004; 322:497-507.
74 Murphy W, Durum S, Anver M, Longo D. Immunologic and hematologic effects of neuroendocrine hormones: Studies on DW/J dwarf mice. J Immunol 1992; 148:3799-3805.
75 Terskikh A, Easterday M, Li Let al. From hematopoiesis to neuropoiesis: Evidence of overlapping genetic programs. Proc Natl Acad Sci USA 2001; 98:7934-7939.
76 Punzel M, Gupta P, Roodell M et al. Factor(s) secreted by AFT024 fetal liver cells following stimulation with human cytokines are important for human LTC-IC growth. Leukemia 1999; 13:1079-1084.
77 Caron K, Smithies O. Extreme hydrops fetalis and cardiovascular abnormalities in mice lacking the adrenomedullin gene. Proc Natl Acad Sci USA 2001; 98:615-619.
78 Harzenetter M, Keller U, Beer S et al. Regulation and function of the CGRP receptor complex in human granulopoiesis. Exp Hematol 2002; 30:306-312.
79 De Angeli S, Di Liddo R, Buoro S et al. New immortalized human stromal cell lines enhancing in vitro expansion of cord blood hematopoietic stem cells. Int J Mol Med 2004; 13:363-371.

### EXAMPLE 4

Increasing levels of exposure to ionizing radiation can cause a spectrum of damage to the skin and the hematopoietic, gastrointestinal, pulmonary, and central nervous systems [1-4]. The hematopoietic and immune systems are among the most sensitive tissues to the adverse effects of ionizing radiation: lymphocyte decline and thrombocytopenia are reported after as low as 50 cGy of exposure [4]. After 400 cGy of exposure, more severe myelosuppression occurs, and the mortality risk is estimated to be 50% in the absence of medical intervention [4]. At doses >400 cGy, bone marrow (BM) failure and death can occur despite maximal supportive care with transfusion support and antibiotics [2-4].

Experimental studies have demonstrated that low-dose ionizing radiation induces cellular apoptosis via activation of Fas ligand-mediated pathways [5, 6], whereas higher-close radiation induces double-stranded DNA damage, which causes necrotic cell death in proliferating cells [7]. It is interesting to note that the administration of interleukin (IL)-1 or stem cell factor (SCF) before or at the time of high-dose radiation exposure protects mice from radiation lethality, thus suggesting that induction of stem/progenitor cells into late S phase of the cell cycle is radioprotective [8, 9]. Alternately, administration of tumor necrosis factor α, which induces production of free-radical scavengers, is also radioprotective [10], although its efficacy is evident primarily after low-dose radiation exposure [11]. Administration of megakaryocyte growth and development factor (MGDF), a ligand for Mpl [12], at the time of high-dose irradiation is also 100% radioprotective in mice and has been shown to inhibit the actions of p53 to prevent radiation-induced apoptosis [12]. The combined administration of SCF, fins-like tyrosine kinase 3 (Flt-3) ligand, thrombopoietin (TPO), IL-3, and stromal cell derived factor 1 (SDF-1) to B6D2F1 mice within 2 hours after 800 cGy has also been shown to support the survival of 87.5% of mice, compared with 8.3% in controls [13]. However, in the event of a nuclear blast or a nuclear power plant accident, the administration of cytokines to victims within 2 hours of exposure will be difficult. Moreover, experimental studies have yielded conflicting results with regard to the potential benefits of cytokine administration when they are administered more than 2 to 4 hours after high-dose radiation exposure [9, 14-18]. For example, Macvittie et al. [14] demonstrated that the administration of 10 μg/kg/d of granulocyte colony-stimulating factor plus supportive care beginning at 20 hours after 500 cGy of total body irradiation was associated with 75% survival of dogs, compared with 0% survival in untreated animals. Conversely, Zsebo et al. [9] demonstrated that administration of 100 μg/kg SCF beginning 4 hours after a lethal dose (1150 cGy) of total body irradiation in mice provided no radioprotection in any animals, and Neelis et al. [16] showed that the radioprotective effects of thrombopoietin were dramatically reduced between 2 and 24 hours after 600 cGy of exposure in mice. Taken together, these data indicate that additional therapies capable of accelerating hematopoietic reconstitution several hours to days after radiation-induced aplasia should be explored.

We examined the capacity of primary vascular endothelial cells (ECs) to support the self-renewal and expansion of murine, primate, and human hematopoietic stem cells (HSCs) [19-21]. In addition to the contribution of osteoblasts in supporting HSCs within the BM niche [22, 23], the potential role of ECs in the BM vascular niche has recently been suggested [24]. We have observed that primary human brain ECs (HUBECs) support, in noncontact culture, a 1 to 2 log expansion of human BM and cord blood (CB) severe combined immunodeficient (SCID)-repopulating cells (SRCs) [25, 26]. We also have observed that HSCs harvested from the BM of lethally irradiated C57BL/6 mice could be functionally rescued via coculture with brain ECs [19]. In this study, we examined whether human HSCs could be rescued from the deleterious effects of ionizing radiation via coculture with primary HUBECs. We found that soluble factors elaborated by HUBECs support the recovery and expansion of irradiated human BM HSCs, whereas treatment with cytokines alone is ineffective.

### Methods

### HUBEC Cultures

HUBECs (passage >10) were developed in primary culture from explanted cortical brain vessel segments (obtained via autopsy specimens from the University of California-Los Angeles Department of Neuropathology) as previously described [21]. These cells highly express human von Willebrand factor, thus indicating an endothelial phenotype (data not shown). Briefly, gelatin-coated 6-well plates (Costar, Cambridge, Mass.) were seeded with 1×10⁵ HUBECs in complete EC medium containing Medium 199 (Invitrogen, Carlsbad, Calif.), 10% heat-inactivated fetal bovine serum (FBS; Hyclone, Logan, Utah), 0.3 mg/mL L-glutamine, 100 U/mL penicillin, 100 μg/mL streptomycin (1% penicillin/streptomycin), 60 mg/L EC growth supplement, and 4.5 U/mL heparin (Sigma, St. Louis, Mo.). HUBECs were cultured for 48 hours to >90% confluence in a 37° C., 5% carbon dioxide atmosphere before the establishment of CD34⁺ cell cocultures.

### Irradiation of Human CD34⁺ Cells and In Vitro Coculture

Cryopreserved human BM CD34⁺ (Cambrex, Gaithersburg, Md.) or CB CD34⁺ cells (AllCells, Berkeley, Calif.) were thawed, washed once, and resuspended at 1×10⁶/mL in Iscove modified Dulbecco medium (IMDM; Invitrogen) containing 10% FBS and 1% penicillin/streptomycin. CD34⁺ cells (>95% purity) were then exposed to 400 cGy in vitro in polystyrene conical tubes (Becton Dickinson, San Jose, Calif.) by using a cesium 137 radiation source. Cells were maintained on ice and placed into culture 2 hours after irradiation. A dose of 400 cGy was used because this is a representative level of exposure that has been estimated to occur after nuclear power plant accidents [27].

Cultures were established with 1 to 2×10⁵ irradiated BM or CB CD34⁺ cells in 6-well plates with media containing IMDM, 10% FBS, 1% penicillin/streptomycin, 20 ng/mL thrombopoietin, 120 ng/mL SCF, and 50 ng/mL fms-like Flt-3 ligand (TSF; R&D Systems, Minneapolis, Minn.). For noncontact HUBEC cocultures, irradiated BM or CB CD34⁺ cells were placed into 0.4-μm polystyrene transwell inserts (Costar). Cultures were maintained in a 37° C., 5% carbon dioxide atmosphere for 10 days, with media supplementation (2 mL per well) at day 7. At day 10, nonadherent cells were collected from the culture by vigorous flushing with warm IMDM containing 10% FBS and 1% penicillin/streptomycin.

### In Vitro Hematopoietic Progenitor Cell Assays

BM CD34⁺ and CB CD34⁺ cells that were irradiated in vitro with 400 cGy were analyzed for immunophenotype at 6 hours after irradiation. Day 0 non-irradiated cells were analyzed as controls. Irradiated cell subsets were also placed in culture with TSF or HUBECs under contact and noncontact conditions approximately 4 hours after irradiation. Day 10 cultured progeny were collected and washed with phosphate-buffered saline (Invitrogen) and resuspended in IMDM with 10% FBS and 1% penicillin/streptomycin. The total viable cell count was determined by hemacytometer count with trypan blue dye exclusion. For phenotype analysis, cells were stained with anti-CD34 fluorescein isothiocyanate and anti-CD38 phycoerythrin or the appropriate immunoglobulin G iso-type control antibodies (Becton Dickinson) for 30 minutes on ice. For apoptosis analysis, cells were stained with anti-annexin (Becton Dickinson) V fluorescein isothiocyanate, anti-CD38 phycoerythrin, and anti-CD34 allophycocyanin for 30 minutes on ice. Cells were washed twice and stained with 7-amino-actinomycin D (7-AAD; Becton Dickinson) for 10 minutes on ice before analysis. Sample acquisition was conducted on a FACScalibur flow cytometer (Becton Dickinson). Statistical comparisons between groups were performed by using the t test.

Colony-forming assays were established in Metho-Cult GF H4434 complete methylcellulose medium (Stem Cell Technologies, Vancouver, Bc, Canada) with 1×10³ cells per dish in 35-mm gridded petri dishes (Nunc, Rochester, N.Y.), according to the manufacturer's recommended protocol. After 14 days, triplicate cultures were scored for burst-forming units-erythroid (BFU-E), colony-forming units-granulocyte monocyte (CFU-GM), and colony-forming unit-mix (CFU-Mix) colony (>50 cells) formation.

### Nonobese Diabetic/SCID Repopulation Assays

Six- to 8-week-old nonobese diabetic/SCID (NOD/SCID) mice [28] underwent transplantation with day 0 400 cGy-irradiated BM CD34⁺ cells (0.75-1.5×10⁶) or their cultured progeny. A subset of mice was also injected with an identical dose of normal, day 0 non-irradiated BM CD34⁺ cells as a positive control. NOD/SCID mice received transplants via tail vein injection after receiving 300 cGy of total body irradiation on an X-Rad 320 irradiation system (AGFA NDT Inc., Lewistown, Pa.) at a dose rate of 100 cGy/min 4 hours before transplantation, as previously described [26]. Eight weeks after transplantation, mice were killed, and marrow was collected from bilateral femurs by flushing with cold Dulbecco's phosphate buffered saline with 10% FBS. Red cells were lysed by using red blood cell lysing buffer (Sigma) and washed twice, and flow cytometric analysis was performed to determine human hematopoietic engraftment by using monoclonal antibodies against human leukocyte differentiation antigens to identify engrafted human leukocytes and discriminate their hematopoietic lineages [21, 29]. Mice were scored as positively engrafted if the BM displayed ≧0.1% human CD45⁺ cells via high-resolution flow cytometry analysis, consistent with previously published criteria for human cell repopulation in NOD/SCID mice [30, 31].

### Results

Contact and Noncontact Culture with HUBECs Increases the Recovery of Irradiated Hematopoietic Progenitors

The combination of thrombopoietin, SCF, and Flt-3 ligand (TSF) has been shown to optimize the in vitro maintenance of CB SRCs [32, 33], and our group has shown that these same cytokines, when combined with HUBECs, maximize the ex vivo expansion of purified BM CD34⁺CD38⁻ SRCs [26]. Therefore, we chose to compare the capacity for TSF alone versus HUBEC plus TSF to support the recovery of BM and CB CD34⁺ cells after irradiation with 400 cGy.

Culture of 400 cGy-irradiated BM CD34⁺ cells with TSF alone supported a 2.8-fold increase in total viable cells compared with day 0 cells; however, a significant decrement in the CD34⁺CD38⁻ subset was observed by day 10 (FIG. 13A, B1. Conversely, HUBEC contact cultures supported an 11.3-fold increase in total cells at day 10 and were associated with a significant increase in the percentage of CD34⁺CD38⁻ cells in culture (mean, 18.2%) compared with TSF alone (mean, 0.5%; P=0.005). This translated into a 29.4-fold increase in CD34⁺CD38⁻ cells in HUBEC contact cultures compared with input, as compared with a 4.9-fold decrease in CD34⁺CD38⁻ cells with cytokines alone. Culture of irradiated BM CD34⁺ cells with HUBECs under noncontact conditions supported a 5.8-fold expansion of total cells and a 4.8-fold increase in the CD34⁺CD38⁻ subset compared with day 0 cells (FIGS. 1A and B). Although this was significantly less than the recovery observed in HUBEC contact cultures, the recovery of total viable cells and CD34⁺CD38⁻ cells in noncontact HUBEC cultures was significantly increased compared with TSF cultures alone (P=0.01 and P=0.01, respectively).

Irradiation of human CB CD34⁺ cells yielded similar results as compared with BM CD34⁺ cells. After a 10-day culture with TSF alone, a 9.2-fold expansion of total cells was observed, but a 3.9-fold decline in CD34⁺CD38⁻ cells occurred by day 10 (FIGS. 1C and D). In contrast, HUBEC contact cultures supported a 29.5-fold increase in total cells and a 28.6-fold increase in CD34⁺CD38⁻ cells compared with input. Noncontact HUBEC cultures supported a 17.7-fold increase in total cells and a 3.9-fold increase in CD34⁺CD38⁻ cells (FIG. 13C, D). The recovery and expansion of total viable cells and CDA⁺CD38⁻ cells was significantly higher in both HUBEC contact and noncontact cultures compared with cultures with TSF alone (P≦0.01). FIG. 14 shows a representative phenotypic analysis of day 0 BM and CB CD34⁺ cells after 400 cGy of irradiation and their progeny after culture with TSF alone and HUBEC contact and noncontact cultures.

Coculture with HUBECs Supports the Recovery of Colony-Forming Cells from Irradiated BM and CB

Colony-forming cell (CFC) assay of normal and 400 cGy-irradiated day 0 BM and CB CD34⁺ cells highlighted the ablative effects of 400 cGy of ionizing radiation on hematopoietic progenitor cell activity (FIG. 15). The 400 cGy-irradiated BM CD34⁺ cells contained 18.4-fold less CFC content (CFU-total; P<0.001) and showed marked reductions in BFU-E (6.9-fold reduction) and CFU-GM (32.7-fold reduction) content and a complete loss of CFU-Mix colonies, as compared with nonirradiated BM CD34⁺ cells. Significant reductions in CFU-total (P=0.008; FIG. 15), BFU-E, CFU-GM, and CFU-Mix content were also observed after irradiation of CB CD34⁺ cells.

Both contact and noncontact HUBEC cultures supported the recovery of CFCs from 400 cGy-irradiated BM CD34⁺ cells at levels significantly greater than TSF alone (3.6-fold and 3.9-fold increased CFU-total, respectively; P<0.001 and P=0.002). HUBEC contact and noncontact cultures also recovered BFU-E and CFU-Mix colonies, which were completely absent from TSF-cultured progeny of irradiated BM CD34⁺ cells (data not shown). HUBEC contact and noncontact cultures of 400 cGy-irradiated CB CD34⁺ cells yielded similar results, with significant increases in CFU-total content (3.2-fold and 3.0-fold; P=0.02 and P<0.005, respectively), as well as BFU-E, CFU-GM, and CFU-Mix (data not shown), as compared with TSF alone (FIG. 15

Coculture with HUBECs Reduces Hematopoietic Progenitor Cell Death After Radiation Exposure

We hypothesized that ECs might elaborate anti-apoptotic factors that could promote the recovery of hematopoietic progenitor cells after radiation injury. Analysis with Annexin V and 7-AAD revealed interesting similarities and differences in the percentage of apoptotic (Annexin V⁺/7-AAD⁻) and necrotic (Annexin V⁺/7-AAD⁺) cells within the nonirradiated BM CD34⁺ cells, the irradiated BM CD34⁺ cells, and the progeny of irradiated BM CD34⁺ cells under different culture conditions. Notably, overall cell death was significantly increased within 400 cGy-irradiated BM CD34⁺ cells measured 6 hours after irradiation as compared with nonirradiated BM CD34⁺ cells (FIG. 16). Analysis of the entire population of irradiated BM cells demonstrated moderately increased apoptosis and necrosis in TSF cultures as compared with both HUBEC contact and noncontact cultures, and this was most evident at day 3 and day 10 (FIG. 16A Within the CD34⁺ progenitor cell subset, TSF culture was associated with a significant increase in cell death over time as compared with both HUBEC contact and noncontact cultures (FIG. 16B

HUBEC Culture Supports the Recovery of Repopulating Stem Cells from Irradiated Human BM CD34⁺ Cells

NOD/SCID mice received transplants via tail vein injection with day 0 normal (nonirradiated), day 0 400 cGy-irradiated, or the progeny of 400 cGy-irradiated BM CD34⁺ cells after a 10-day culture with HUBECs or TSF alone. We observed that exposure to 400 cGy of ionizing radiation had a profoundly deleterious effect on the repopulating capacity of BM CD34⁺cells. Mice that underwent transplantation with a dose of 0.75×10⁶ nonirradiated BM CD34⁺ cells demonstrated low-level (mean, 0:1% human CD45⁺ cells) engraftment in 50% of transplanted animals (FIG. 17, Conversely, mice that underwent transplantation with day 0 400 cGy-irradiated BM CD34⁺ cells or their day 10 progeny after culture with TSF alone demonstrated no human cell engraftment. Mice that underwent transplantation with the progeny of 0.75×10⁶ 400 cGy-irradiated BM CD34⁺ cells after HUBEC contact culture also showed no human CD45⁺ cell engraftment ≧0.1%, although a single mouse had 0.02% human CD45⁺ cells at 8 weeks after transplantation.

At a dose of 1.5×10⁶ nonirradiated BM CD34⁺cells, 100% of transplanted mice demonstrated human CD45⁺ cell engraftment at high levels (mean, 36.8% human CD45⁺ cells). Conversely, mice that underwent transplantation with 400 cGy-irradiated BM CD34⁺ cells showed human CD45⁺ cell engraftment in 75% of animals, with significantly lower levels of engraftment (mean, 1.0% human CD45⁺ cells; FIG. 17 This indicates that a small population of SRCs was able to survive 400 cGy of radiation injury. It is interesting to note that the progeny of the identical dose of 400 cGy-irradiated BM CD34⁺ cells cultured with TSF alone for 10 days were incapable of engrafting and repopulating any transplanted mice; this suggests that cytokine treatment was insufficient and possibly deleterious toward the survival of primitive long-term repopulating stem cells after high-dose radiation exposure. In contrast, the progeny of 1.5×10⁶ 400 cGy-irradiated BM CD34⁺ cells cultured under non-contact conditions with HUBECs engrafted in 100% of transplanted mice with a mean engraftment level of 3.5% human CD45⁺ cells per mouse, thus demonstrating that soluble endothelial factors promoted the recovery of irradiated human HSCs independently of cell-cell contact.

Representative phenotypic analyses of human CD45⁺ cell frequencies in mice that underwent trans-plantation with nonirradiated BM CD34⁺ cells, irradiated BM CD34⁺ cells, and the progeny of 400 cGy-irradiated BM CD34⁺ cells after culture with TSF alone versus noncontact HUBEC culture are shown in FIG. 18A. Of note, mice that underwent transplantation with the progeny of 400 cGy-irradiated BM CD34⁺ cells cultured with HUBECs under noncontact conditions demonstrated multilineage (B lymphoid and myeloid) engraftment, thus indicating that multipotent stem/progenitor cells were maintained after irradiation and HUBEC coculture (FIG. 18B). Of note, the proportion of B lymphoid regeneration in mice that underwent transplantation with irradiated/HUBEC-cultured cells was comparatively higher than the observed regeneration of CD13⁺ myeloid progeny, and this suggests a potentially important difference with regard to the native recovery of B lymphoid progenitors versus myeloid progenitors after high-dose irradiation.

### Discussion

Recently, because of the acknowledged risk of nuclear or radiological terrorism over the coming decade, there has been renewed interest in the development of medical countermeasures to the effects of ionizing radiation exposure [1-4]. Therapies directed at ameliorating the hematologic toxicity of ionizing radiation would be of particular interest because BM failure is the leading cause of death in victims of pure ionizing radiation injury [1-4, 34, 35]. In animal models, administration of cytokines such as SCF, MGDF, Flt-3 ligand, IL-1, or tumor necrosis factor α before or immediately at the time of high-dose total body radiation exposure can provide radioprotection and improve survival [8-12, 16-18]. Similarly, the combined administration of multiple cytokines, including SCF, Flt-3 ligand, MGDF, IL-3, and SDF-1, within 2 hours after sublethal and near-lethal irradiation has been associated with decreased myelosuppression and improved survival in mice and baboons [13, 36]. However, in the event of a radiologic or nuclear catastrophe, the administration of hematopoietic cytokines will not be feasible for many within the first 2 hours of exposure, and it is unclear whether the administration of cytokines more than a few hours after exposure would be therapeutically valuable after higher-dose exposures. SCF, for example, has no effect on radiation-induced myelosuppression and mortality when administered only 4 hours after high-dose radiation exposure [9], and granulocyte colony-stimulating factor, which clearly accelerates myeloid recovery in primates after sublethal exposures [37-40], has no effect when administered to mice after an exposure of 10.5 Gy [40]. Although we did not measure the capacity for HUBEC coculture to rescue human HSCs irradiated in vitro with doses >400 cGy, we have recently demonstrated that fully functional BM stem and progenitor cells can be rescued after harvest from lethally irradiated (1050 cGy of total body irradiation) C57BL/6 mice via coculture with porcine brain ECs [19]. Because few studies have been performed to characterize the regenerative capacity of human HSCs after ionizing radiation exposure, we propose that the HUBEC coculture model has the potential to yield important insights regarding EC-derived factors that may be radioprotective.

In this study, we showed that primary human BM CD34⁺ cells are exquisitely sensitive to 400 cGy of exposure in vitro, thus resulting in dramatic declines in CFC and SRC content after injury. Treatment with hematopoietic cytokines alone was associated with an increase in apoptosis and necrosis of irradiated BM CD34⁺ cells and a marked decline in CD34⁺CD38⁻ cells and SRCs compared with input, despite treatment within 4 hours of exposure, thus suggesting that cytokine treatment of human BM HSCs after high-dose irradiation may, in fact, be deleterious to their recovery. These results are in contrast to the observations of Drouet et al. [27], who reported that early treatment of baboon BM CD34⁺ cells with SCF, Flt-3 ligand, thrombopoietin, and IL-3 decreased Fas ligand-mediated apoptosis in vitro. However, in that study, the authors did not examine the long-term repopulating potential of the irradiated baboon cells after cytokine treatment, so conclusions regarding the effect of cytokines on baboon HSCs cannot be drawn [27]. In addition, ionizing radiation induces mammalian cells, in general, to undergo either cell-cycle arrest or apoptosis in the immediate postexposure period [41, 42]. Cells that undergo p53-independent cell-cycle arrest in G₁ or G₂/M phase have the potential to repair radiation-induced DNA damage and avoid cell death [43]. Because the cytokine combination of thrombopoietin, SCF, and Flt-3 ligand has been shown to induce nearly 100% of human CB CD34⁺ cells through at least 1 cell division by 1 week of culture [33], it is plausible that exposure of irradiated BM CD34⁺ cells to these proliferation-inducing cytokines accelerates the demise of stem and progenitor cells. Conversely, HUBEC contact cultures and, to a lesser extent, HUBEC noncontact cultures decreased radiation-induced apoptosis and necrosis of BM CD34⁺ cells and promoted a significant increase in the recovery of total viable cells, CD34⁺CD38⁻ cells, and CFCs as compared with TSF alone. These results are consistent with prior studies that suggested that that cell-cell contact interactions (eg, Jagged-Notch) between HSCs and other stromal cell types are critical to HSC survival [22, 23, 44].

It is interesting to note that although HUBEC contact cultures supported a greater recovery of total viable cells and CD34⁺CD38⁻ cells than non-contact cultures, HUBEC noncontact cultures supported a potent recovery of the most primitive SRCs. These data can be potentially explained, as others have shown [31], by the lack of correlation between stem cell content and phenotypic indicators after ex vivo culture. It is also possible that coculture with HUBECs induced cell-cycle arrest in HSCs as a mechanism of radioprotection, as has been described when murine embryonic neural stem cells were cultured in contact with murine brain ECs [45]. However, we have found that coculture with porcine brain ECs induces the proliferation of BM HSCs harvested from lethally irradiated mice [19], thus suggesting that a true expansion of radioprotected HSCs occurs via this strategy.

It is important to note that our results demonstrate that soluble factors produced by ECs support the survival and regeneration of human HSCs after radiation injury. Our previous analyses have shown that established hematopoietic cytokines, including SCF, Flt-3 ligand, thrombopoietin, granulocyte colony-stimulating factor, granulocyte-macrophage colony-stimulating factor, and IL-3, are not enriched within HUBEC conditioned media [46]. This suggests that potentially novel prosurvival factors elaborated by HUBECs account for the effects we have observed. This soluble activity may be unique to vascular ECs as well. Recent studies by Mourcin et al. [47] and Drouet et al. [48] demonstrated that coculture with mesenchymal stem cells supported the recovery of 400 cGy-irradiated baboon CD34⁺ cells, but this was dependent on cell-cell contact between the irradiated cells and the mesenchymal stem cells. We are currently pursuing identification of the HUBEC-soluble proteins that are responsible for the observed radiotherapeutic effects, via complementary genomic and protein fractionation strategies. We anticipate that the identification and characterization of these soluble proteins may facilitate the development of therapeutics to counteract radiation-induced myelosuppression.

The results presented here suggest that hematopoietic progenitor cells could, in principle, be collected from radiation-accident victims, expanded ex vivo, and transplanted in an autologous manner to accelerate the hematopoietic recovery of such victims. However, in the event of a mass casualty situation, such an approach would not be logistically feasible. Nonetheless, these data suggest an important contribution of vascular ECs to the repair and regeneration of human BM stem and progenitor cells after radiation injury. In other disease models, such as myocardial infarction and peripheral vascular disease, the anti-apoptotic activity of circulating ECs has been proposed [49]. In addition, Kopp et al. [50] have shown the potential contribution of the BM vascular niche and angiogenic factors toward accelerating hematopoietic recovery after myelosuppressive chemotherapy. Targeted therapies aimed at accelerating the recovery of the BM vascular endothelial niche may therefore be of benefit in the treatment of victims of radiation injury. As proof of principle, we have recently observed that tail vein transplantation of primary vascular ECs is radioprotective and accelerates hematopoietic recovery in lethally irradiated mice (Chute J, unpublished data).

A recent workshop on radiation countermeasures sponsored by the National Institute of Allergy and Infectious Diseases concluded that the current lack of effective therapeutic agents for the treatment of radiation victims is a major problem in the government's preparation for radiologic or nuclear catastrophes [1]. More broadly, newly developed therapies that accelerate hematopoietic recovery after radiation injury could also have application in attenuating the myelotoxic effects of chemotherapy and radiotherapy in patients with cancer. Our results indicate that human vascular ECs elaborate soluble factors that support the repair and recovery of irradiated human stem and progenitor cells. The characterization of these novel factors has the potential to lead to targeted therapies for radiation-induced myelosuppression.

### REFERENCES

1. Augustine A D, Gondre-Lewis T, McBride W, Miller L, Pellmar T, Rockwell S. Animal models for radiation injury, protection and therapy. Radios Res. 2005; 164:100-109.
2. Wasalenko J, Waselenko J, Macvittie T, et al. Medical management of the acute radiation syndrome: recommendations of the Strategic National Stockpile Radiation Working Group. Ann Intern Med. 2004; 140:1037-1051.
3. Mettler F, Voelz G. Major radiation exposure—what to expect and how to respond. N Engl j Med. 2002; 346:1554-1561.
4. Dainiak N. Hematologic consequences of exposure to ionizing radiation. Exp Hematol. 2002; 30:513-528.
5. Albanese J, Dainiak N. Ionizing radiation alters Fas ligand at the cell surface and on exfoliated plasma membrane-derived vesicles: implications for apoptosis and intercellular signaling. Radiat Res. 2000; 153:49-61.
6. Belka C, Marini P, Budach W, et al. Radiation induced apoptosis in human lymphocytes and lymphoma cells critically relies on the up-regulation of CD9/Fas/APO-1 Ligand. Radiat Res. 1998; 149:588-595.
7. Radford I, Murphy T. Radiation response of mouse lymphoid and myeloid cell lines. Part III. Different signals can lead to apoptosis and may influence sensitivity to killing by DNA double-strand breakage. Int J Radiat Biol. 1994; 65:229-239.
8. Neta R, Oppenheim J, Wang J, et al. Synergy of IL-1 and SCF in radioprotection of mice is associated with IL-1 upregulation of mRNA and protein expression for c-kit on bone marrow cells. J Immunol. 1994; 153:1536-1543.
9. Zsebo K, Smith K, Hartley C, et al. Radioprotection of mice by recombinant stem cell factor. Proc Natl Acad Sri USA. 1992; 89:9464-9468.
10. Neta R. Modulation with cytokines of radiation injury: suggested mechanism of action. Environ Health Persp. 1995; 107(suppl 6):1463-1465.
11. Karkanitsa L, Komarovskaya M, Krivenko S. Abrogation of radiation injury to human hematopoietic stem cells with rumor necrosis factor-alpha. Stem Cells. 1997; 15(suppl 2):95-102.
12. Pestina T, Cleveland J, Yang C, Zambetti G, Jackson C. Mpl ligand prevents lethal myelosuppression by inhibiting p53-dependent apoptosis. Blood. 2001; 98:2084-2090.
13. Herodin F, Bourin P, Mayol J, Lataillade J, Drouet M. Short term injection of antiapoprotic cytokine combinations soon after lethal γ-irradiation promotes survival. Blood. 2003; 101: 2609-2616.
14. Macvittie T, Farese A, Jackson W. Defining the full therapeutic potential of recombinant growth factors in the post radiation accident environment: the effect of supportive care plus administration of recombinant G-CSF. Health Phys. 2005; 89:546-555.
15. Farese A, Macvittie T, Roskos L, Stead R. Hematopoietic recovery following autologous bone marrow transplantation in a non-human primate: effect of variation of treatment schedule with PEG-rHuMGDF. Stem Cells. 2003; 21:79-89.
16. Neelis K, Visser T, Dimjati W, et al. A single dose of thrombopoietin shortly after myelosuppressive total body irradiation prevents pancytopenia in mice by promoting short-term multilineage spleen repopulating cells at the transient expense of bone marrow repopulating cells. Blood. 1998; 92:1586-1597.
17. Hudak S, Leach M, Xu Y, Menon S, Rennick D. Radioprotective effects of flk2/flt3 ligand. Exp Hematol. 1998; 26:515-522.
18. Mouthon M, Van der Meeren A, Gaugler M, et al. Thrombopoietin promotes hematopoietic recovery and survival after high-dose whole body irradiation. Int J Radiat Oncol Biol Phys. 1999; 43:867-875.
19. Chute J, Fung J, Muramoto G, Erwin R. Ex vivo culture rescues hematopoietic stern cells with long term repopulating capacity following harvest from lethally irradiated mice. Exp 2004; 32:308-317.
20. Brandt J, Bartholemew A, Fortman J, et al. Ex vivo expansion of autologous bone marrow CD34⁺ cells with porcine microvascular endothelial cells results in a graft capable of rescuing lethally irradiated baboons. Blood. 1999; 94:106-113.
21. Chute J, Saini A, Chute D, et al. Ex vivo culture with human brain endothelial cells increases the SCID-repopulating capacity of adult human bone marrow. Blood. 2002; 100:4433-4439.
22. Zhang J, Niu C, Ye L, et al. Identification of the haematopoietic stem cell niche and control of the niche size. Nature. 2003; 425:836-841.
23. Calvi L, Adams G, Weibrecht K, et al. Osteoblastic cells regulate the haematopoietic stem cell niche. Nature. 2003; 425:841-846.
24. Avecilla S, Hattori K, Heissig B, et al. Chemokine mediated interaction of hematopoietic progenitors with the bone marrow vascular niche is required for thrombopoiesis. Nat Med. 2004; 10:64-71.
25. Chute J P, Fungi, Muramoto G, Oxford C. Quantitative analysis demonstrates expansion of SCID-repopulating cells (SRC) and increased engraftment capacity within human cord blood cells following culture with human brain endothelial cells. Stem Cells. 2004; 22:202-215.
26. Chute J P, Muramoto G, Fung J, Oxford C. Soluble factors elaborated by human brain endothelial cell induce the concomitant expansion of purified BM CD34⁺CD38⁻ cells and SCID repopulating cells. Blood. 2005; 105:576-583.
27. Drouet M, Mathieu J, Grenier N, Multon E, Sotto J, Herodin F. The reduction of in vitro radiation induced Fes-related apoptosis in CD34⁺ progenitor cells by SCF, Flt-3 ligand, TPO and IL-3 in combination resulted in CD34⁺ cell proliferation and differentiation. Stem Cells. 1999; 17:273-285.
28. Schulz L, Schweitzer P, Christianson S, et al. Multiple defects in innate and adaptive immunologic function in NOD/LtSz-said mice. J Immunol. 1995; 154:180-191.
29. Trischmann T, Schepers K, Civin C. Measurement of CD34⁺ cells in bone marrow by flow cytometry. J Hematother. 1993; 2: 305-313.
30. Glimm H, Eaves C. Direct evidence for multiple self-renewal divisions of human in vivo repopulating cells in short-term culture. Blood. 1999; 94:2161-2168.
31. Dorrell C, Gan O, Pereira D, Hawley R, Dick J. Expansion of human cord blood CD34⁺CD38⁻ cells in ex vivo culture during retroviral transduction without a corresponding increase in SCID repopulating cell (SRC) frequency: disiociation of SRC phenotype and function. Blood. 2000; 95:102-110.
32. Piacibello W, Sanavio F, Severino A, et al. Engraftment in nonobese diabetic severe combined immunodeficient mice of human CD34⁺ cord blood cells after ex vivo expansion: evidence for the amplification and self-renewal of repopulating stem cells. Blood. 1999; 93:3736-3749.
33. Tanavde V, Malehorn M, Lumkul R, et al. Human stem-progenitor cells from neonatal cord blood have greater hematopoietic expansion capacity than those from mobilized adult blood. Exp Hematol. 2002; 30:816-823.
34. Gale R. Immediate medical consequences of nuclear accidents. Lessons from Chernobyl. JAMA. 1987; 258:625-628.
35. Baranov A, Gale R, Guskova A, et al. Bone marrow transplantation after the Chernobyl nuclear accident. N Engl J Med. 1989; 321:205-212.
36. Drouet M, Mourcin F, Grenier N, et al. Single administration of stem cell factor, FLT-3 ligand, megakaryocyte growth and development factor, and interleukin-3 in combination soon after irradiation prevents nonhuman primates from myelosuppression: long-term follow up of hematopoiesis. Blood. 2004; 103:878-885.
37. Farese A, Hunt P, Grab L, MacVittie T. Combined administration of recombinant human megakaryocyte growth and development factor and granulocyte colony stimulating factor enhances multilineage hematopoietic reconstitution in nonhuman primates after radiation-induced marrow aplasia. J Clin Invest. 1996; 97:2145-2151.
38. Farese A, Casey D, Vigneulle R, et al. A single dose of pegylated leridistim significantly improves neutrophil recovery in sublethally irradiated rhesus macaques. Stem Cells. 2001; 19:514-521.
39. Farese A, Casey D, Smith W, et al. Leridistim, a chimeric dual GCSF and IL-3 receptor agonist, enhances multilineage hematopoietic recovery in a nonhuman primate model of radiation induced myelosuppression: effect of schedule, dose and route of administration. Stem Cells. 2001; 19:522-533.
40. Tanikawa S, Nose M, Aoki Y, et al. Effects of recombinant granulocyte colony stimulating factor on the hematologic recovery and survival of irradiated mice. Blood. 1990; 76:445-449.
41. Quelle F, Wang J, Feng J, et al. Cytokine rescue of p53-dependent apoptosis and cell cycle arrest is mediated by distinct Jak kinase signaling pathways. Genes Dev. 1998; 12:1099-1107.
42. Henry M, Lynch J, Eapen A, Quelle F. DNA damage induced cell cycle arrest of hematopoietic cells is overridden by activation of the P1-3 kinase/Akt signaling pathway. Blood. 2001; 98:834-841.
43. Campanini F, Santucci M, Brusa G, et al. Expression of p21 (WAF1/CIP1/SID1) cyclin dependent kinase inhibitor in hematopoietic progenitor cells. Gene. 2001; 273:173-180.
44. Tsai S, Fero J, Bartelmez S. Mouse jagged 2 is differentially expressed in hematopoietic progenitors and endothelial cells and promotes the survival and proliferation of hematopoietic progenitors by direct cell-to-cell contact. Blood. 2000; 96:950-957,
45. Mathieu C, Fouchet P, Gauthier L, Lassalle B, Boussin F, Mouthon M. Coculture with endothelial cells reduces the population of cycling Let; neural precursors but increases that of quiescent cells with a side population phenotype. Exp Cell Res. 2006; 312:707-718.
46. Chute J, Clark W, Johnson K, Saini A, Wells M, Harlan D. Hematopoietic cytokine production by human brain endothelial cells (HUBEC) is associated with the ex vivo expansion of CD34+CD38− progenitor cells. Exp Hematol. 2000; 28:96a.
47. Mourcin F, Grenier N, Mayol J, et al. Mesenchymal stem cells support expansion of in vitro irradiated CD34⁺ cells in the presence of SCF, Flt-3 ligand, TPO and IL-3: potential application to autologous cell therapy in accidentally irradiated victims. Radiat. Res. 2005; 164:1-9.
48. Drouet M, Mourcin F, Grenier N, et al. Mesenchymal stem cells rescue CD34⁺ cells from radiation-induced apoptosis and sustain hematopoietic reconstitution after coculture and cografting in lethally irradiated baboons: is autologous stem cell therapy in nuclear accident settings hype or reality? Bone Marrow Transplant. 2005; 35:1201-1209.
49. Kong D, Melo L, Gnecchi M, et al. Cytokine induced mobilization of circulating endothelial progenitor cells enhances repair of injured arteries. Circulation. 2004; 110:239-246.
50. Kopp H, Avecilla S, Hooper. A, et al. Tie-2 activation contributes to hemangiogenic revascularization after myelosuppression. Blood. 2005; 106:505-513.

All documents and other information sources cited above are hereby incorporated in their entirety by reference. Also incorporated by reference is Muramoto et al, Biol. Blood Marrow Transplant. 12(5):530-540 (2006).

What is claimed is:

1. A method of stimulating expansion of hematopoietic stem cells (HSC) comprising contacting said cells with an amount of at least one protein, or portion thereof, listed in Table 3, 5 or 7 sufficient to effect said stimulation.

2. The method according to claim 1 wherein said HSC are human HSC.

3. The method according to claim 1 wherein said HSC are CD34+CD38−.

4. The method according to claim 1 wherein said HSC are derived from bone marrow.
